(19) United States
(12) Patent Application Publication
(10) Pub. No.: US 2013/0034536 A1
(43) Pub. Date: Feb. 7, 2013

(54) Bile Acid Recycling Inhibitors for Treatment of Pancreatitis

(71) Applicant: Bronislava GEDULIN, Del Mar, CA (US)

(72) Inventor: Bronislava GEDULIN, Del Mar, CA (US)

(73) Assignee: Lumena Pharmaceuticals, Inc., Durham, NC (US)

(21) Appl. No.: 13/566,898

(22) Filed: Aug. 3, 2012

Related U.S. Application Data
(60) Provisional application No. 61/553,086, filed on Oct. 28, 2011.
(60) Provisional application No. 61/515,293, filed on Aug. 4, 2011.

(51) Int. Cl. A61K 31/554; A61K 31/155; A61K 38/02; A61K 31/38; A61K 31/4985; A61K 31/4436; A61K 31/7042; A61K 31/7052; A61K 31/56; A61K 31/57; A61K 31/575; A61K 31/506; A61K 31/42; A61K 31/55; A61K 31/167; A61P 1/18; A61P 29/00; A61K 38/48

(52) U.S. Cl. 424/94.64; 514/635; 514/1.1; 514/211.09; 514/431; 514/249; 514/337; 514/42; 514/27; 514/211.08; 514/169; 514/182; 514/256; 514/378; 514/619; 514/215; 514/621

(57) ABSTRACT

Provided herein are methods and compositions comprising bile acid transport inhibitors and/or enteroendocrine peptide enhancing agents and/or FXR agonists for the treatment of pancreatitis or prevention of pancreatitis.

### CROSS-REFERENCE TO RELATED APPLICATIONS

This application claims the benefit under 35 U.S.C. §119(e) of U.S. Provisional Application No. 61/515,293, filed Aug. 4, 2011, and U.S. Provisional Application No. 61/553,086, filed Oct. 28, 2011, which are incorporated herein by their entirety.

### BACKGROUND OF THE INVENTION

Pancreatitis is an inflammation of the pancreas that causes severe abdominal pain. An estimated 50,000 to 80,000 cases of acute pancreatitis occur in the U.S. each year. Most cases in the U.S. are caused either by alcohol abuse or by gallstones. Other causes may be use of prescription drugs, trauma or surgery to the abdomen, or abnormalities of the pancreas or intestine. In rare cases, the disease may result from viral infections, such as mumps. In about 15% of cases, the cause is unknown. If injury to the pancreas continues, chronic pancreatitis may develop subsequent to acute pancreatitis. Severe pancreatitis can have serious consequences, including malnutrition, diabetes, kidney failure and death. An effective treatment of pancreatitis is needed.

### SUMMARY OF THE INVENTION

Described herein are compositions and methods for treatment or prevention of pancreatitis that involve the use of an ASBT inhibitor (ASBTI) or a pharmaceutically acceptable salt thereof, an enteroendocrine peptide enhancing agent or a pharmaceutically acceptable salt thereof, or a nuclear farnesoid X receptor (FXR) agonist or a pharmaceutically acceptable salt thereof, or a combination thereof, to modulate pancreatic secretions and/or activation of pancreatic enzymes. In certain embodiments, the methods provided herein comprise non-systemically administering an ASBT inhibitor (ASBTI) or a pharmaceutically acceptable salt thereof, an enteroendocrine peptide enhancing agent or a pharmaceutically acceptable salt thereof, or a nuclear farnesoid X receptor (FXR) agonist or a pharmaceutically acceptable salt thereof, or a combination thereof. In some embodiments, the methods provided herein comprise administration of a non-systemically absorbed compound selected from an ASBTI or a pharmaceutically acceptable salt thereof, an enteroendocrine peptide enhancing agent or a pharmaceutically acceptable salt thereof, a nuclear farnesoid X receptor (FXR) agonist or a pharmaceutically acceptable salt thereof, and a combination thereof. In some embodiments, the methods provided herein comprise administration of a non-systemically absorbed formulation comprising an ASBTI or a pharmaceutically acceptable salt thereof, an enteroendocrine peptide enhancing agent or a pharmaceutically acceptable salt thereof, or a nuclear farnesoid X receptor (FXR) agonist or a pharmaceutically acceptable salt thereof, or a combination thereof. In some embodiments, compositions and methods provided herein decrease pancreatic secretions and/or activation of pancreatic enzymes.

In one aspect, provided herein are compositions and methods for reducing pancreatic enzyme activity comprising administration of an ASBT inhibitor (ASBTI) or a pharmaceutically acceptable salt thereof, an enteroendocrine peptide enhancing agent or a pharmaceutically acceptable salt thereof, or a nuclear farnesoid X receptor (FXR) agonist or a pharmaceutically acceptable salt thereof, or a combination thereof, to an individual suffering from pancreatitis.

In some embodiments, provided herein are compositions and methods for reducing the secretion or the activity of amylase, lipase, and/or other pancreatic proteases comprising administration of an ASBT inhibitor (ASBTI) or a pharmaceutically acceptable salt thereof, an enteroendocrine peptide enhancing agent or a pharmaceutically acceptable salt thereof, or a nuclear farnesoid X receptor (FXR) agonist or a pharmaceutically acceptable salt thereof, or a combination thereof.

In some embodiments, provided herein are compositions and methods for treating or preventing pancreatic injury comprising administration of an ASBT inhibitor (ASBTI) or a pharmaceutically acceptable salt thereof, an enteroendocrine peptide enhancing agent or a pharmaceutically acceptable salt thereof, or a nuclear farnesoid X receptor (FXR) agonist or a pharmaceutically acceptable salt thereof, or a combination thereof. In one aspect, compositions and methods described herein increase intraluminal concentrations of bile acids in an individual in need thereof. In some embodiments, increased intraluminal bile acid concentrations according to methods described herein protect and/or restore the integrity of an individual's pancreas when the pancreas has been injured by inflammation and/or hyperactivation of pancreatic enzymes.

In one aspect, provided herein are compositions and methods for increasing the levels of a pancreatic peptide or hormone or an enteroendocrine peptide or hormone in an individual in need thereof comprising administration of an ASBT inhibitor (ASBTI) or a pharmaceutically acceptable salt thereof, an enteroendocrine peptide enhancing agent or a pharmaceutically acceptable salt thereof, or a nuclear farnesoid X receptor (FXR) agonist or a pharmaceutically acceptable salt thereof, or a combination thereof. In some embodiments, compositions and methods described herein protect and/or restore the integrity of an individual's pancreas when the pancreas has been injured by inflammation and/or hyperactivation of pancreatic enzymes. In some embodiments, the pancreatic peptide or hormone is amylin or insulin. In some embodiments, the enteroendocrine peptide or hormone is glucagon-like peptide 1 (GLP-1), GLP-2, peptide tyrosine-tyrosine (PYY), and/or oxyntomodulin (OXM).

Provided herein are methods and compositions for use in the treatment of pancreatitis in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

Provided herein are methods and compositions for use in the treatment of pancreatic inflammation in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

Also provided herein are methods and compositions for use in the treatment of pain associated with pancreatitis in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

Provided herein in another aspect are methods and compositions for use in the prevention of acute and/or chronic pancreatitis comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof. In some embodiments, provided herein are methods and compositions for use in the prevention of acute and/or chronic pancreatitis after a surgical pancreato-biliary intervention or procedure. In some embodiments, the surgical pancreato-biliary intervention or procedure is pancreatic resection, Endoscopic Retrograde Cholangiopancreatography Procedure (ERCP), gallbladder surgery, bile duct surgery, liver surgery, liver transplantation, or bariatric surgery.

In yet another aspect, provided herein are methods and compositions for prevention of acute pancreatitis as a complication of an Endoscopic Retrograde Cholangiopancreatography Procedure (ERCP) in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

In some embodiments, the methods and compositions described herein further comprise administration of a second agent selected from a liver receptor homolog 1 (LRH-1), a DPP-IV inhibitor, a proton pump inhibitor, H2 antagonist, prokinetic agent, a biguanide, an incretin mimetic, a mucoadhesive agent, GLP-1 or an analog thereof, and a pancreatic enzyme.

In some embodiments, any of the methods and compositions described herein further comprise administration of a pain relieving medication.

In one embodiment, provided herein is a method for treating or preventing (e.g., in an individual who has undergone a pancreato-biliary procedure) pancreatitis in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof.

In another aspect, provided herein is a method for treating or preventing (e.g., in an individual who has undergone a pancreato-biliary procedure) pancreatitis in an individual in need thereof comprising non-systemically administering to the individual in need thereof a therapeutically effective amount of an enteroendocrine peptide enhancing agent a pharmaceutically acceptable salt thereof.

In a further aspect, provided herein is a method for treating or preventing (e.g., in an individual who has undergone a pancreato-biliary procedure) pancreatitis in an individual in need thereof comprising non-systemically administering to the individual in need thereof a therapeutically effective amount of an FXR agonist a pharmaceutically acceptable salt thereof.

In some embodiments, any of the methods or compositions described herein reduce or ameliorate symptoms of pancreatitis and/or reduce severity of symptoms and/or reduce recurrence of pancreatitis. In some embodiments, for any of the methods and/or compositions described herein, the individual is an individual who has undergone a pancreato-biliary surgical procedure.

Provided herein, in certain embodiments, are therapeutic methods and compositions using compounds that inhibit the Apical Sodium-dependent Bile Transporter (ASBT) or a pharmaceutically acceptable salt thereof, or any recuperative bile salt transporter for treatment of pancreatitis and/or pain associated with pancreatitis. In certain instances, use of the compounds provided herein reduces or inhibits recycling of bile acid salts in the gastrointestinal tract. In some embodiments, the methods provided herein reduce intraenterocyte bile acids and/or damage to pancreas caused by inflammation and/or auto-digestion. In some embodiments, less than 50%, less than 40%, less than 30%, less than 20%, less than 10%, less than 9%, less than 8%, less than 7%, less than 6%, less than 5%, less than 4%, less than 3%, less than 2%, or less than 1% of the ASBTI and/or the enteroendocrine peptide enhancing agent and/or a FXR agonist is systemically absorbed. In some embodiments, the ASBT inhibitors provided herein are non-systemic compounds. In some embodiments, the ASBT inhibitors provided herein are minimally absorbed systemically. In some embodiments, less than 10% the ASBT inhibitors provided herein are absorbed systemically. In certain embodiments, the ASBT inhibitors described herein enhance L-cell secretion of enteroendocrine peptides.

In some embodiments, the ASBTI provided herein is a compound of Formula I or a pharmaceutically acceptable salt thereof, as described herein. In some embodiments, the ASBTI provided herein is a compound of Formula II or a pharmaceutically acceptable salt thereof, as described herein. In some embodiments, the ASBTI provided herein is a compound of Formula III or a pharmaceutically acceptable salt thereof, as described herein. In some embodiments, the ASBTI provided herein is a compound of Formula IV or a pharmaceutically acceptable salt thereof, as described herein. In some embodiments, the ASBTI provided herein is a compound of Formula V or a pharmaceutically acceptable salt thereof, as described herein. In some embodiments, the ASBTI provided herein is a compound of Formula VI or Formula VID or a pharmaceutically acceptable salt thereof, as described herein.

In certain embodiments, an ASBTI is any compound described herein that inhibits recycling of bile acids in the gastrointestinal tract of an individual. In certain embodiments, an ASBTI is (−)-(3R,5R)-trans-3-butyl-3-ethyl-2,3,4,5-tetrahydro-7,8-dimethoxy-5-phenyl-1,4-benzothiazepine1,1-dioxide; (“Compound 100A”) or any other salt or analog thereof. In certain of any of the aforementioned embodiments, an ASBTI is 1-[4-[4-[(4R,5R)-3,3-dibutyl-7-(dimethylamino)-2,3,4,5-tetrahydro-4-hydroxy-1,1-dioxido-1-benzothiepin-5-yl]phenoxy]butyl]4-aza-1-azoniabicyclo[2.2.2]octane methane sulfonate salt (“Compound 100B”) or any other salt or analog thereof. In certain embodiments, an ASBTI is N,N-dimethylimido-dicarbonimidic diamide (“Compound 100C”) or any salt or analog thereof. In certain embodiments, an ASBTI is any commercially available ASBTI including but not limited to SD-5613, A-3309, 264W94, S-8921, SAR-548304, BARI-1741, HMR-1453, TA-7552, R-146224, or SC-435. In some embodiments, an ASBTI is 1-[[5-[[3-[(3S,4R,5R)-3-butyl-7-(dimethylamino)-3-ethyl-2,3,4,5-tetrahydro-4-hydroxy-1,1-dioxido-1-benzothiepin-5-yl]phenyl]amino]-5-oxopentyl]amino]-1-deoxy-D-glucitol; or Potassium((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl)sulphate ethanolate, hydrate. In certain embodiments, an ASBTI is 264W94 (Glaxo), SC-435 (Pfizer), or A3309 (Astra-Zeneca). In certain embodiments, an ASBTI provided herein is not a compound disclosed in WO12/064,266, which is incorporated by reference herein.

Provided herein, in certain embodiments, are therapeutic methods and compositions using compounds that are enteroendocrine peptide secretion enhancing agents for treatment of pancreatitis and/or pain associated with pancreatitis. In certain instances, use of the compounds provided herein reduces or inhibits recycling of bile acid salts in the gastrointestinal tract. In some embodiments, the methods provided herein reduce intraenterocyte bile acids and/or damage to pancreas caused by inflammation and/or auto-digestion. In some embodiments, the enteroendocrine peptide secretion enhancing agents provided herein are non-systemic compounds. In some embodiments, the enteroendocrine peptide secretion enhancing agents provided herein are minimally absorbed systemically. In some embodiments, less than 10% the enteroendocrine peptide secretion enhancing agents provided herein are absorbed systemically. In certain embodiments, the enteroendocrine peptide secretion enhancing agents described herein enhance L-cell secretion of enteroendocrine peptides.

In certain embodiments, an enteroendocrine peptide secretion enhancing agent is a bile acid, a bile salt, a bile acid mimic, a bile salt mimic, TGR5 agonist, or a combination thereof. In some embodiments, the enteroendocrine peptide secretion enhancing agent is a glucagon-like peptide secretion enhancing agent, optionally in combination with a bile acid, a bile salt, a bile acid mimic, or a bile salt mimic. In certain embodiments, the glucagon-like peptide secretion enhancing agent is a glucagon-like peptide-1 (GLP-1) secretion enhancing agent, or a glucagon-like peptide-2 (GLP-2) secretion enhancing agent, optionally in combination with a bile acid, a bile salt, a bile acid mimic, or a bile salt mimic. In some embodiments, the enteroendocrine peptide secretion enhancing agent is a pancreatic polypeptide-fold peptide secretion enhancing agent, optionally in combination with a bile acid, a bile salt, a bile acid mimic, or a bile salt mimic. In some embodiments, the pancreatic polypeptide-fold peptide secretion enhancing agent is a peptide YY (PYY) secretion enhancing agent.

In certain embodiments, a bile acid mimetic is a TGR5 agonist, M-BAR agonist, GPR119 agonist, GPR120 agonist, GPR131 agonist, GPR140 agonist, GPR143 agonist, GPR53 agonist, GPBAR1 agonist, BG37 agonist, farnesoid-X receptor agonist. In some instances, a bile acid mimetic promotes L-cell secretions. In certain instances, a bile acid mimetic promotes the secretion of GLP-1, GLP-2, PYY, OXM, or a combination thereof.

Provided herein, in certain embodiments, are therapeutic methods and compositions using compounds that are FXR agonists for treatment of pancreatitis and/or pain associated with pancreatitis. In certain instances, use of the compounds provided herein reduces or inhibits recycling of bile acid salts in the gastrointestinal tract. In some embodiments, the methods provided herein reduce intraenterocyte bile acids and/or damage to pancreas caused by inflammation and/or auto-digestions. In some embodiments, the FXR provided herein agonists are non-systemic compounds. In some embodiments, the FXR agonists provided herein are minimally absorbed systemically. In some embodiments, less than 10% the FXR agonists provided herein are absorbed systemically. In certain embodiments, the FXR agonists described herein enhance L-cell secretion of enteroendocrine peptides.

In certain embodiments, the FXR agonist is GW4064, GW9662, INT-747, T0901317, WAY-362450, fexaramine, a cholic acid, a deoxycholic acid, a glycocholic acid, a glycodeoxycholic acid, a taurocholic acid, a taurodihydrofusidate, a taurodeoxycholic acid, a cholate, a glycocholate, a deoxycholate, a taurocholate, a taurodeoxycholate, a chenodeoxycholic acid, an ursodeoxycholic acid, a tauroursodeoxycholic acid, a glycoursodeoxycholic acid, a 7-B-methyl cholic acid, a methyl lithocholic acid, or a salt thereof, or a combination thereof.

Provided in certain embodiments herein are methods and dosage forms (e.g., oral or rectal dosage form) for use in the treatment of pancreatitis and symptoms thereof, comprising a therapeutically effective amount of an ASBTI, or a pharmaceutically acceptable salt thereof, and a carrier. In some embodiments, provided herein is a method for treating pancreatitis and symptoms thereof comprising orally administering a therapeutically effective amount of a minimally absorbed ASBTI, or a pharmaceutically acceptable salt thereof, to an individual in need thereof.

In certain embodiments, the ASBTI, or salt thereof is a minimally absorbed ASBTI. In some embodiments, the dosage form is an enteric formulation, an ileal-pH sensitive release formulation, or a suppository or other suitable form.

Provided in certain embodiments herein are methods and dosage forms (e.g., oral or rectal dosage form) for use in the treatment of pancreatitis and symptoms thereof comprising a therapeutically effective amount of a bile acid, bile salt, or mimetic thereof, and a carrier. In some embodiments, provided herein is a method for treating pancreatitis and symptoms thereof comprising rectally administering a therapeutically effective amount of a minimally absorbed bile acid, bile acid salt, or mimetic thereof, to an individual in need thereof.

In certain embodiments, the bile acid, bile salt, or mimetic thereof is a minimally absorbed bile acid, bile salt, or mimetic thereof. In some embodiments, the dosage form is an enteric formulation, an ileal-pH sensitive release, or a suppository or other suitable form.

In some embodiments, a composition for use in treatment of pancreatitis and/or symptoms thereof described above comprises at least one of a spreading agent or a wetting agent. In some embodiments, the composition comprises an absorption inhibitor. In some cases an absorption inhibitor is a mucoadhesive agent (e.g., a mucoadhesive polymer). In certain embodiments, the mucoadhesive agent is selected from methyl cellulose, polycarbophil, polyvinylpyrrolidone, sodium carboxymethyl cellulose, and combinations thereof. In some embodiments, the enteroendocrine peptide secretion enhancing agent is covalently linked to the absorption inhibitor.

In certain embodiments, the carrier is a rectally suitable carrier. In certain embodiments, any pharmaceutical composition described herein is formulated as a suppository, an enema solution, a rectal foam, or a rectal gel. In some embodiments, any pharmaceutical composition described herein comprises an orally suitable carrier. In certain embodiments, the pharmaceutical composition comprises an enteric coating.

In some embodiments, provided herein is a pharmaceutical composition formulated for non-systemic ileal, rectal or colonic delivery of the ASBTI and/or enteroendocrine peptide secretion enhancing agent and/or FXR agonist.

In some embodiments, for any of the methods described herein, administration of an ASBTI and/or an enteroendocrine peptide enhancing agent and/or a FXR agonist reduces intraenterocyte bile acids in an individual in need thereof. In some embodiments, the methods described herein reduce accumulation of bile acids in ileal enterocytes of an individual in need thereof. In some embodiments, for any of the methods described herein, administration of an ASBTI and/or an enteroendocrine peptide enhancing agent and/or a FXR agonist inhibits transport of bile acids from ileal lumen into enterocytes of an individual in need thereof. In some embodiments, for any of the methods described herein, administration of an ASBTI and/or an enteroendocrine peptide enhancing agent and/or a FXR agonist increases ileal luminal bile acids in an individual in need thereof. In some embodiments, for any of the methods described herein, administration of an ASBTI and/or an enteroendocrine peptide enhancing agent and/or a FXR agonist reduces damage to pancreas caused by inflammation and/or auto-digestion in an individual in need thereof. In some embodiments, for any of the methods described herein, administration of an ASBTI and/or an enteroendocrine peptide enhancing agent and/or a FXR agonist reduces pancreatic secretions and/or production of inflammatory cytokines that are associated with onset of pancreatitis in an individual in need thereof.

In some embodiments, the methods and compositions described herein further comprise administration of one or more agents selected from a liver receptor homolog 1 (LRH-1), a DPP-IV inhibitor, a proton pump inhibitor, H2 antagonist, prokinetic agent, a biguanide, an incretin mimetic, a mucoadhesive agent, GLP-1 or an analog thereof, a TGR5 agonist, a pain medication, and a pancreatic enzyme. By way of example, in one case, a composition or method of treating pancreatitis comprises administration of a bile acid mimetic, a DPP-IV inhibitor, and a pain therapeutic to an individual in need thereof. In another example, a composition or method of treating pancreatitis comprises administration of an ASBTI, a DPP-IV inhibitor, and a pain therapeutic, and further, optionally, a pancreatic enzyme to an individual in need thereof.

In some embodiments, the methods provided herein further comprise administering a therapeutically effective amount of an inhibitor of Dipeptidyl Peptidase-4. In some embodiments, the inhibitor of Dipeptidyl Peptidase-4 is administered orally or rectally. In some embodiments, the inhibitor of Dipeptidyl Peptidase-4 is co-administered with an ASBTI, an enteroendocrine peptide enhancing agent, a FXR agonist, bile acid, bile salt, or mimetic thereof. In some embodiments, the inhibitor of Dipeptidyl Peptidase-4 is an absorbable or systemically absorbed inhibitor of Dipeptidyl Peptidase-4.

In some embodiments, the methods and compositions described above further comprise administration of a second agent selected from a liver receptor homolog 1 (LRH-1), a DPP-IV inhibitor, a proton pump inhibitor, H2 antagonist, prokinetic agent, a biguanide, an incretin mimetic, a mucoadhesive agent, GLP-1 or an analog thereof, and a TGR5 agonist. In some embodiments, the second agent is a DPP-IV inhibitor.

In some embodiments, the methods and compositions described above further comprise administration of a pain medication. In some embodiments, the methods and compositions described above further comprise administration of a pancreatic enzyme.

In some embodiments, provided herein are methods for the treatment of pancreatitis and/or symptoms thereof (e.g., pain) comprising administration of a therapeutically effective amount of a combination of an ASBTI and a DPP-IV inhibitor to an individual in need thereof. In some embodiments, provided herein are methods for the treatment of pancreatitis and/or symptoms thereof (e.g., pain) comprising administration of a therapeutically effective amount of a combination of an ASBTI and a TGR5 agonist to an individual in need thereof. In some embodiments, provided herein are methods for the treatment of pancreatitis and/or symptoms thereof (e.g., pain) comprising administration of a therapeutically effective amount of a combination of an ASBTI and GLP-1 or an analog thereof to an individual in need thereof. In some embodiments, provided herein are methods for the treatment of pancreatitis and/or symptoms thereof (e.g., pain) comprising administration of a therapeutically effective amount of a combination of an ASBTI and a biguanide to an individual in need thereof. In some embodiments, provided herein are methods for the treatment of pancreatitis and/or symptoms thereof (e.g., pain) comprising administration of a therapeutically effective amount of a combination of an ASBTI and a pain medication to an individual in need thereof. In some embodiments, provided herein are methods for the treatment of pancreatitis and/or symptoms thereof (e.g., pain) comprising administration of a therapeutically effective amount of a combination of an ASBTI and a pancreatic enzyme to an individual in need thereof. In some embodiments, provided herein are methods for the treatment of pancreatitis and/or symptoms thereof (e.g., pain) comprising administration of a therapeutically effective amount of a combination of an ASBTI and one or more of a pain medication, a DPP-IV inhibitor, and a pancreatic enzyme to an individual in need thereof.

In some embodiments, the ASBTI and/or the enterendocrine peptide enhancing agent and/or the FXR agonist is administered orally. In some embodiments, the ASBTI and/or the enterendocrine peptide enhancing agent and/or the FXR agonist is administered as an ileal-pH sensitive release formulation that delivers the ASBTI and/or the enterendocrine peptide enhancing agent and/or the FXR agonist to the distal ileum, colon and/or rectum of an individual. In some embodiments, the ASBTI and/or the enterendocrine peptide enhancing agent and/or the FXR agonist is administered as an enterically coated formulation. In some embodiments, oral delivery of an ASBTI and/or an enterendocrine peptide enhancing agent and/or a FXR agonist provided herein can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action (the ileum) by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

In some embodiments of the methods described above, the ASBTI and/or the enterendocrine peptide enhancing agent and/or the FXR agonist is administered before ingestion of food. In some embodiments of the methods described above, the ASBTI and/or the enterendocrine peptide enhancing agent and/or the FXR agonist is administered with or after ingestion of food.

Provided in some embodiments herein is a kit comprising any composition described herein (e.g., a pharmaceutical composition formulated for rectal administration) and a device for localized delivery within the rectum or colon. In certain embodiments, the device is a syringe, bag, or a pressurized container.

### BRIEF DESCRIPTION OF THE DRAWINGS

FIG. 1 depicts the effects of bile salt transporter inhibitor SC-435 on plasma active GLP-1 levels, which increased levels are associated with treatment and prevention of pancreatitis.

FIG. 2 depicts the effects of bile acid, taurocholate, on plasma active GLP-1 levels, which increased levels are associated with treatment and prevention of pancreatitis.

### DETAILED DESCRIPTION OF THE INVENTION

The human pancreas secretes over a liter of enzyme and zymogen containing fluid per day as part of its role in the major digestive activity of the gastrointestinal tract. Regulation of pancreatic secretion is by both hormonal and neural mechanisms, with the former being of primary importance. The secreted enzymes include trypsin, amylases, lipases, and/or other proteolytic enzymes, which may be packaged in precursor form or in combination with inhibitors to prevent autodigestion of pancreatic cells. Enzyme secretion is also regulated in part by a negative feedback mechanism induced by enzyme and/or enteropeptide hormone levels in the gastrointestinal tract.

Pancreatitis is an inflammatory disease which is clinically diagnosed as acute or chronic. Acute pancreatitis is a complex clinical condition that ranges in severity from mild to life-threatening. Abdominal pain, ultrasound-confirmed pancreatic pathological changes, and increased plasma amylase and lipase concentrations are the most common markers of acute pancreatitis in the clinic.

The cellular functions and molecular mechanisms responsible for initiating and modifying the severity of pancreatitis have not been fully elucidated. In general, acinar cells, which secrete digestive enzymes into pancreatic ducts, play an important role in the development of pancreatitis. A common feature in manifestation of pancreatitis is the premature activation of trypsinogen within pancreatic tissues, which triggers autodigestion of the gland. Pancreatic injury likely occurs by auto-digestion of the pancreas via retention of hyper-activated digestive enzymes followed by a highly amplified inflammatory response, edema, cellular damage and necrosis.

Acute pancreatitis is characterized by edema, acinar cell necrosis, hemorrhage, and severe inflammation of the pancreas. Patients with acute pancreatitis present with elevated blood and urine levels of pancreatic digestive enzymes, such as amylase and lipase. Severe acute pancreatitis may lead to systemic inflammatory response syndrome and multiorgan dysfunction syndrome, which accounts for the high mortality rate of acute pancreatitis. Although most (>80%) cases of acute pancreatitis are associated with gallstones and alcoholism, some are idiopathic.

When pancreatic enzymes and toxins released during acute pancreatitis gain access to the systemic circulation via retroperitoneal, lymphatic and/or venous pathways, they can affect capillaries and generally cause harmful systemic effects. Respiratory distress syndrome, renal failure and/or heart failure are the most frequent causes of death in patients with acute pancreatitis.

If injury to the pancreas continues, such as when a subject persists in drinking alcohol, a chronic form of the disease may develop, bringing severe pain and reduced functioning of the pancreas that affects digestion and causes weight loss. Chronic pancreatitis may also result from other causes, many of which are also known to induce acute pancreatitis. While pain is also often seen in chronic pancreatitis, the pain may be continuous or intermittent or absent.

Generally, therapeutic approaches used to date against pancreatitis have not been clinically successful. Current therapies aim to 1) prevent passage of nutrients from the stomach into the duodenum (such as by nasogastric suction and intravenous alimentation); 2) prevent acid from entering the duodenum (which normally prompts secretin release and results in pancreatic stimulation; it should be noted that cimetidine to limit acid secretion has not been shown useful in treating pancreatitis); 3) block enzymatic secretion, e.g., with anticholinergic drugs; and 4) inhibit protease activity with aprotinin (Traysylol™), which has been shown to be ineffective in practice. Other approaches include treating pain (e.g., by administration of narcotics), maintaining circulatory function, preventing secondary infection, and eventually, in chronic cases, correction of malabsorption. Thus there is a need for effective therapies for treatment of pancreatitis.

Accordingly, provided herein is a novel approach to treatment of pancreatitis. In certain embodiments, methods and compositions described herein are directed to modifying secretion of pancreatic enzymes by modulating (e.g., increasing) bile acid levels in the gastrointestinal (GI) tract. Such modification of bile acid levels in the GI tract induces changes in levels of circulating enteroendocrine peptides and/or cytokines and also affects the negative feedback mechanism induced by enzyme levels in the alimentary canal, and thus reduces auto-digestion of the pancreas (e.g., due to hyper-activation of pancreatic enzymes such as trypsin, amylases and lipases) which is associated with onset of pancreatitis.

In one aspect, the compositions and methods provided herein increase bile acid concentrations in the gut. Bile acids play a critical role in activating digestive enzymes and solubilizing fats and fat-soluble vitamins and are involved in liver, biliary, and intestinal disease. Formed in the liver, bile acids are absorbed actively from the small intestine, with each molecule undergoing multiple enterohepatic circulations before being excreted. A small percentage of bile salts may be reabsorbed in the proximal intestine by either passive or carrier-mediated transport processes. Most bile salts are reclaimed in the distal ileum by a sodium-dependent apically located bile acid transporter referred to as apical sodium-dependent bile acid transporter (ASBT). At the basolateral surface of the enterocyte, a truncated version of ASBT is involved in vectorial transfer of bile acids into the portal circulation. Completion of the enterohepatic circulation occurs at the basolateral surface of the hepatocyte by a transport process that is primarily mediated by a sodium-dependent bile acid transporter. Without being limited to a particular theory, the increased concentrations of bile acids provided by compositions and methods provided herein stimulate subsequent secretion of factors that affect secretion of pancreatic enzymes.

In yet another aspect, the compositions and methods described herein have an advantage over systemically absorbed agents. The compositions and methods described herein utilize ASBT inhibitors and/or enteroendocrine peptide enhancing agents that are not systemically absorbed or minimally absorbed systemically; thus the compositions are effective without leaving the gut lumen, thereby reducing any toxicity and/or side effects associated with systemic absorption.

In one aspect, compositions and methods described herein stimulate the release of pancreatic hormones, including but not limited to, amylin or insulin.

In a further aspect, the compositions and methods described herein stimulate the release of enteroendocrine hormones, including but not limited to, GLP-1, GLP-2, OXM, and/or PYY. Increased secretion of GLP-1, GLP-2, OXM, or PYY allows for modifying the negative feedback mechanism that is responsible for regulation of pancreatic secretions.

Described herein is the use of inhibitors of the Apical Sodium-dependent Bile Transporter (ASBT) or any recuperative bile salt transporter that are active in the gastrointestinal (GI) tract for treating pancreatitis in an individual in need thereof. In certain embodiments, the methods provided herein comprise administering a therapeutically effective amount of an ASBT inhibitor (ASBTI) and/or an enteroendocrine peptide enhancing agent and/or a FXR agonist to an individual in need thereof. In some embodiments, such ASBT inhibitors and/or enteroendocrine peptide enhancing agents and/or FXR agonists are not systemically absorbed or minimally absorbed systemically. In some embodiments, such bile salt transport inhibitors include a moiety or group that prevents, reduces or inhibits the systemic absorption of the compound in vivo. In some embodiments, a charged moiety or group on the compounds prevents, reduces or inhibits the compounds from leaving the gastrointestinal tract and reduces the risk of side effects due to systemic absorption. In some other embodiments, such ASBT inhibitors and/or enteroendocrine peptide enhancing agents and/or FXR agonists are systemically absorbed. In some embodiments, the ASBTI and/or an enteroendocrine peptide enhancing agent and/or a FXR agonist are formulated for delivery to the distal ileum. In some embodiments, an ASBTI and/or an enteroendocrine peptide enhancing agent and/or a FXR agonist is minimally absorbed. In some embodiments, an ASBTI and/or an enteroendocrine peptide enhancing agent and/or a FXR agonist is non-systemically administered to the colon or the rectum of an individual in need thereof.

In some embodiments, less than 50%, less than 40%, less than 30%, less than 20%, less than 10%, less than 9%, less than 8%, less than 7%, less than 6%, less than 5%, less than 4%, less than 3%, less than 2%, or less than 1% of the ASBTI and/or the enteroendocrine peptide enhancing agent and/or a FXR agonist is systemically absorbed. In certain embodiments, ASBTIs described herein inhibit scavenging of bile salts by recuperative bile acid salt transporters in the distal gastrointestinal tract (e.g., the distal ileum, the colon and/or the rectum).

In some instances, the inhibition of bile salt recycling results in higher concentrations of bile acids or salts in the lumen of the distal gastrointestinal tract or portions thereof (e.g., the distal small bowel and/or colon and/or rectum). As used herein, the distal gastrointestinal tract includes the region from the distal ileum to the anus. In some embodiments, the compounds described herein reduce intraenterocyte bile acids or accumulation thereof. In certain embodiments, the higher concentration of bile salts in the distal small bowel and/or colon and/or rectum modulates (e.g., enhances) the secretion of enteroendocrine peptides in the distal gastrointestinal tract. In some embodiments, the compounds described herein enhance the secretion of enteroendocrine peptides (e.g., GLP-1, GLP-2, oxyntomodulin, PYY, or a combination thereof) from L-cells that are present in the distal ileum, colon and/or the rectum.

In some embodiments, provided herein are methods for delivering bile acids (endogenously or exogenously) to the colorectal area to stimulate secretion of factors that are important for treatment and/or prevention of pancreatitis. Bile acids are active ligands for enteroendocrine cell receptors which activate L-cell secretion of four regulatory peptides: glucagon-like peptide 1 (GLP-1), peptide tyrosine-tyrosine (PYY), oxyntomodulin (OXM) and GLP-2. GLP-1 is the active incretin that stimulates endocrine pancreatic secretion of insulin and amylin. GLP-1 and amylin both act as potent regulators of exocrine pancreas secretion and also play a role in reducing pancreatic amylase and lipase activity and pancreatic cytokine levels.

Provided herein are methods and compositions for increasing GLP-1 levels in the blood and/or plasma and/or the GI tract. In some embodiments, increased secretion of GLP-1 modulates secretion of pancreatic enzymes and/or feedback loops associated with pancreatic secretions thereby reducing hyperactivation of pancreatic enzymes and reducing pancreatic cytokine levels.

Provided herein are methods and compositions for increasing levels of amylin in the blood and/or plasma and/or the GI tract. In some embodiments, increased secretion of amylin modulates secretion of pancreatic enzymes and/or feedback loops associated with pancreatic secretions thereby reducing hyper-activation of pancreatic enzymes and reduces pancreatic cytokine levels.

### Compounds

In some embodiments, provided herein are ASBT inhibitors that reduce or inhibit bile acid recycling in the distal gastrointestinal (GI) tract, including the distal ileum, the colon and/or the rectum. In certain embodiments, the ASBTIs are systemically absorbed. In certain embodiments, the ASBTIs are not systemically absorbed. In some embodiments, ASBTIs described herein are modified or substituted (e.g., with a -L-K group) to be non-systemic. In certain embodiments, any ASBT inhibitor is modified or substituted with one or more charged groups (e.g., K) and optionally, one or more linker (e.g., L), wherein L and K are as defined herein.

In some embodiments, an ASBTI suitable for the methods described herein is a compound of Formula I:

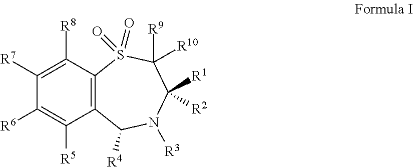

wherein:
R¹ is a straight chained C₁₋₆alkyl group;
R² is a straight chained C₁₋₆alkyl group;
R³ is hydrogen or a group OR¹¹ in which R¹¹ is hydrogen, optionally substituted C₁₋₆alkyl or a C₁₋₆ alkylcarbonyl group;
R⁴ is pyridyl or optionally substituted phenyl or -L_{z}K_{z}; wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
R⁵, R⁶, R⁷ and R⁸ are the same or different and each is selected from hydrogen, halogen, cyano, R⁵-acetylide, OR¹⁵, optionally substituted C₁₋₆alkyl, COR¹⁵, CH(OH)R¹⁵, S(O)ₙR¹⁵, P(O)(OR¹⁵)₂, OCOR¹⁵, OCF3, OCN, SCN, NHCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂R¹⁵, OCH₂OR¹⁵, OCH═CHR¹⁵, O(CH₂CH₂O)ₙR¹⁵, O(CH₂)ₚSO₃R¹⁵, O(CH₂)ₚNR¹²R¹³, O(CH₂)ₚN⁺R¹²R¹³R¹⁴ and —W—R³¹, wherein W is O or NH and R³¹ is selected from

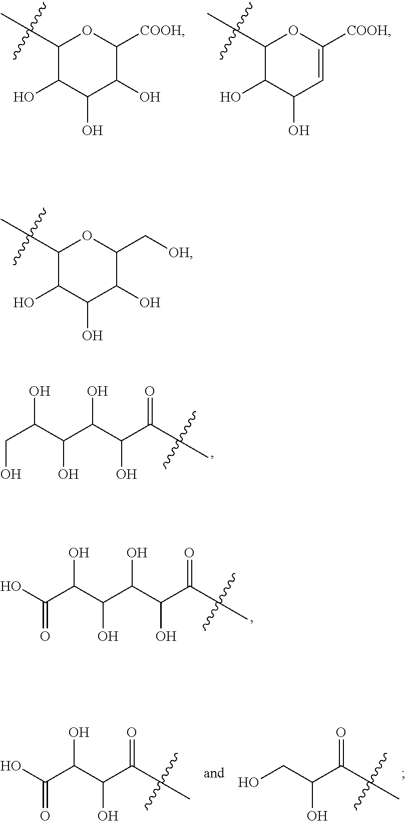

wherein p is an integer from 1-4, n is an integer from 0-3 and, R¹², R¹³, R¹⁴ and R¹⁵ are independently selected from hydrogen and optionally substituted C₁₋₆alkyl; or
   R⁶ and R⁷ are linked to form a group

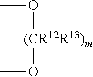

wherein R¹² and R¹³ are as hereinbefore defined and m is 1 or 2; and
   R⁹ and R¹⁰ are the same or different and each is selected from hydrogen or C₁₋₆alkyl; and
   salts, solvates and physiologically functional derivatives thereof.

In some embodiments of the methods, the compound of Formula I is a compound

wherein
R¹ is a straight chained C₁₋₆alkyl group;
R² is a straight chained C₁₋₆alkyl group;
R³ is hydrogen or a group OR¹¹ in which R¹¹ is hydrogen, optionally substituted C₁₋₆alkyl or a C₁₋₆ alkylcarbonyl group;
R⁴ is optionally substituted phenyl;
R⁵, R⁶ and R⁸ are independently selected from hydrogen, C₁₋₄alkyl optionally substituted by fluorine, C₁₋₄ alkoxy, halogen, or hydroxy;
R⁷ is selected from halogen, cyano, R¹⁵-acetylide, OR⁵, optionally substituted C₁₋₆alkyl, COR¹⁵, CH(OH)R¹⁵, S(O)ₙR¹⁵, P(O)(OR¹⁵)₂, OCOR¹⁵, OCF₃, OCN, SCN, HNCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂R¹⁵, OCH₂OR¹⁵, OCH═CHR¹⁵, O(CH₂CH₂O)R¹³, O(CH₂)ₚSO₃R¹⁵, O(CH₂)ₚNR¹²R¹³ and O(CH₂)ₚN⁺R¹²R¹³R¹⁴;

wherein n, p and R¹² to R¹⁵ are as hereinbefore defined;

with the proviso that at least two of R⁵ to R⁸ are not hydrogen; and
salts solvates and physiologically functional derivatives thereof.

In some embodiments of the methods described herein, the compound of Formula I is a compound

wherein
R¹ is a straight chained C₁₋₆alkyl group;
R² is a straight chained C₁₋₆alkyl group;
R³ is hydrogen or a group OR¹¹ in which R¹¹ is hydrogen, optionally substituted C₁₋₆alkyl or a C₁₋₆ alkylcarbonyl group;
R⁴ is un-substituted phenyl;
R⁵ is hydrogen or halogen;
R⁶ and R⁸ are independently selected from hydrogen, C₁₋₄alkyl optionally substituted by fluorine, C₁₋₄alkoxy, halogen, or hydroxy;
R⁷ is selected from OR¹⁵, S(O)ₙR¹⁵, OCOR¹⁵, OCF₃, OCN, SCN, CHO, OCH₂OR¹⁵, OCH═CHR¹⁵, O(CH₂CH₂O)ₙR¹⁵, O(CH₂)ₚSO₃R¹⁵, O(CH₂)ₚNR¹²R¹³ and O(CH₂)ₚN⁺R¹²R¹³R¹⁴ wherein p is an integer from 1-4, n is an integer from 0-3, and R¹², R¹³, R¹⁴, and R¹⁵ are independently selected from hydrogen and optionally substituted C₁₋₆alkyl;
R⁹ and R¹⁰ are the same or different and each is selected from hydrogen or C₁₋₆alkyl; and
salts, solvates and physiologically functional derivatives thereof.

In some embodiments of the methods, wherein the compound of Formula I is a compound

wherein
R¹ is methyl, ethyl or n-propyl;
R² is methyl, ethyl, n-propyl, n-butyl or n-pentyl;
R³ is hydrogen or a group OR¹¹ in which R¹¹ is hydrogen, optionally substituted C₁₋₆alkyl or a C₁₋₆ alkylcarbonyl group;
R⁴ is un-substituted phenyl;
R⁵ is hydrogen;
R⁶ and R⁸ are independently selected from hydrogen, C₁₋₄alkyl optionally substituted by fluorine, C₁₋₄ alkoxy, halogen, or hydroxy;
R⁷ is selected from OR¹⁵, S(O)ₙR¹⁵, OCOR¹⁵, OCF₃, OCN, SCN, CHO, OCH₂OR¹⁵, OCH═CHR¹⁵, O(CH₂CH₂O)ₙR¹⁵, O(CH₂)ₚSO₃R¹⁵, O(CH₂)ₚNR¹²R¹³ and O(CH₂)ₚN⁺R¹²R¹³R¹⁴ wherein p is an integer from 1-4, n is an integer from 0-3, and R¹², R¹³, R¹⁴, and R¹⁵ are independently selected from hydrogen and optionally substituted C₁₋₆alkyl;
R⁹ and R¹⁰ are the same or different and each is selected from hydrogen or C₁₋₆alkyl; and
salts, solvates and physiologically functional derivatives thereof.

In some embodiments of the methods, the compound of Formula I is a compound

wherein
R¹ is methyl, ethyl or n-propyl;
R² is methyl, ethyl, n-propyl, n-butyl or n-pentyl;
R³ is hydrogen or a group OR¹¹ in which R¹¹ is hydrogen, optionally substituted C₁₋₆alkyl or a C₁₋₆ alkylcarbonyl group;
R⁴ is un-substituted phenyl;
R⁵ is hydrogen;
R⁶ is C₁₋₄alkoxy, halogen, or hydroxy;
R⁷ is OR¹⁵, wherein R¹⁵ is hydrogen or optionally substituted C₁₋₆alkyl;
R⁸ is hydrogen or halogen;
R⁹ and R¹⁰ are the same or different and each is selected from hydrogen or C₁₋₆alkyl; and
salts, solvates and physiologically functional derivatives thereof.

In some embodiments of the methods, the compound of Formula I is
(3R,5R)-3-Butyl-3-ethyl-2,3,4,5-tetrahydro-7,8-dimethoxy-5-phenyl-1,4-benzothiazepine 1,1-dioxide;
(3R,5R)-3-Butyl-3-ethyl-2,3,4,5-tetrahydro-7,8-dimethoxy-5-phenyl-1,4-benzothiazepin-4-ol 1,1-dioxide;
(±)-Trans-3-butyl-3-ethyl-2,3,4,5-tetrahydro-7,8-dimethoxy-5-phenyl-1,4-benzothiazepine 1,1-dioxide;
(±)-Trans-3-butyl-3-ethyl-2,3,4,5-tetrahydro-7,8-dimethoxy-5-phenyl-1,4,-benzothiazepin-4-ol 1,1-dioxide;
(3R,5R)-7-Bromo-3-butyl-3-ethyl-2,3,4,5-tetrahydro-8-methoxy-5-phenyl-1,4-benzothiazepine 1,1-dioxide;
(3R,5R)-7-Bromo-3-butyl-3-ethyl-2,3,4,5-tetrahydro-8-methoxy-5-phenyl-1,4-benxothiaxepin-4-ol 1,1-dioxide;
(3R,5R)-3-Butyl-3-ethyl-2,3,4,5-tetrahydro-5-phenyl-1,4-benzothiazepine-7,8-diol 1,1-dioxide;
(3R,5R)-3-Butyl-3-ethyl-2,3,4,5-tetrahydro-8-methoxy-5-phenyl-1,4-benzothiazepin-7-ol 1,1-dioxide;
(3R,5R)-3-Butyl-3-ethyl-2,3,4,5-tetrahydro-7-methoxy-5-phenyl-1,4-benzothiazepin-8-ol 1,1-dioxide;
(±)-Trans-3-butyl-3-ethyl-2,3,4,5-tetrahydro-8-methoxy-5-phenyl-1,4-benzothiazepine 1,1-dioxide;
(±)-Trans-3-butyl-3-ethyl-2,3,4,5-tetrahydro-5-phenyl-1,4-benzothiazepin-8-ol 1,1-dioxide;
(±)-Trans-3-butyl-3-ethyl-2,3,4,5-tetrahydro-5-phenyl-1,4-benzothiazepine-4,8-diol;
(±)-Trans-3-butyl-3-ethyl-2,3,4,5-tetrahydro-5-phenyl-1,4-benzothiazepin-8-thiol 1,1-dioxide;
(±)-Trans-3-butyl-3-ethyl-2,3,4,5-tetrahydro-5-phenyl-1,4-benzothiazepin-8-sulfonic acid 1,1-dioxide;
(±)-Trans-3-butyl-3-ethyl-2,3,4,5-tetrahydro-8,9-dimethoxy-5-phenyl-1,4-benzothiazepine 1,1-dioxide;
(3R,5R)-3-butyl-7,8-diethoxy-2,3,4,5-tetrahydro-5-phenyl-1,4-benzothiazepine 1,1-dioxide;
(±)-Trans-3-butyl-8-ethoxy-3-ethyl-2,3,4,5-tetrahydro-5-phenyl-1,4-benzothiazepine 1,1-dioxide;
(±)-Trans-3-butyl-3-ethyl-2,3,4,5-tetrahydro-8-isopropoxy-5-phenyl-1,4-benzothiazepine 1,1-dioxide hydrochloride;
(±)-Trans-3-butyl-3-ethyl-2,3,4,5-tetrahydro-5-phenyl-1,4-benzothiazepin-8-carbaldehyde-1,1-dioxide;
3,3-Diethyl-2,3,4,5-tetrahydro-7,8-dimethoxy-5-phenyl-1,4-benzothiazepine 1,1-dioxide;
3,3-Diethyl-2,3,4,5-tetrahydro-8-methoxy-5-phenyl-1,4-benzothiazepine 1,1-dioxide;
3,3-Diethyl-2,3,4,5-tetrahydro-5-phenyl-1,4-benzothiazpin-4,8-diol 1,1-dioxide;
(RS)-3,3-Diethyl-2,3,4,5-tetrahydro-4-hydroxy-7,8-dimethoxy-5-phenyl-1,4-benzothiazepine 1,1-dioxide;
(±)-Trans-3-butyl-8-ethoxy-3-ethyl-2,3,4,5-tetrahydro-5-phenyl-1,4-benzothiazepin-4-ol-1-dioxide;
(±)-Trans-3-butyl-3-ethyl-2,3,4,5-tetrahydro-8-isopropoxy-5-phenyl-1,4-benzothiazepin-4-ol 1,1-dioxide;
(±)-Trans-3-butyl-3-ethyl-2,3,4,5-tetrahydro-7,8,9-trimethoxy-5-phenyl-1,4-benzothiazepin-4-ol 1,1-dioxide;
(3R,5R)-3-butyl-3-ethyl-2,3,4,5-tetrahydro-5-phenyl-1,4-benzothiazepin-4,7,8-triol 1,1-dioxide;
(±)-Trans-3-butyl-3-ethyl-2,3,4,5-tetrahydro-4,7,8-trimethoxy-5-phenyl-1,4-benzothiazepine 1,1-dioxide;
3,3-Diethyl-2,3,4,5-tetrahydro-5-phenyl-1,4-benzothiazepin-8-ol 1,1-dioxide;
3,3-Diethyl-2,3,4,5-tetrahydro-7-methoxy-5-phenyl-1,4-benzothiazepin-8-ol 1,1-dioxide;
3,3-Dibutyl-2,3,4,5-tetrahydro-5-phenyl-1,4-benzothiazepin-8-ol 1,1-dioxide;
(±)-Trans-3-Butyl-3-ethyl-2,3,4,5-tetrahydro-1,1-dioxo-5-phenyl-1,4-benzothiazepin-8-yl hydrogen sulfate; or
3,3-Diethyl-2,3,4,5-tetrahydro-1,1-dioxo-5-phenyl-1,4-benzothiazepin-8-yl hydrogen sulfate.

In some embodiments, the compound of Formula I is

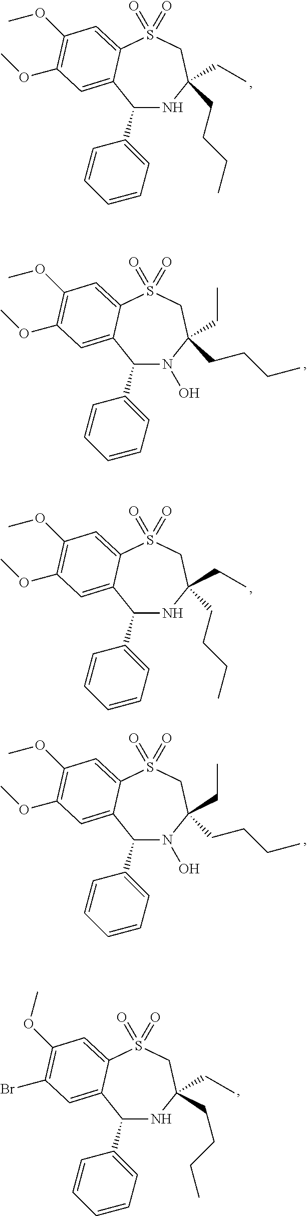

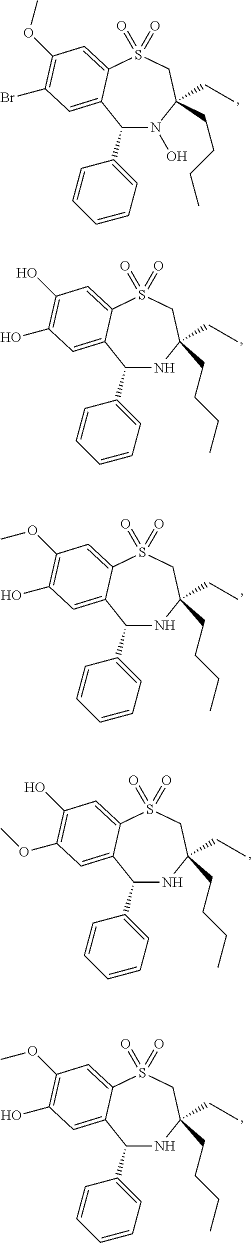

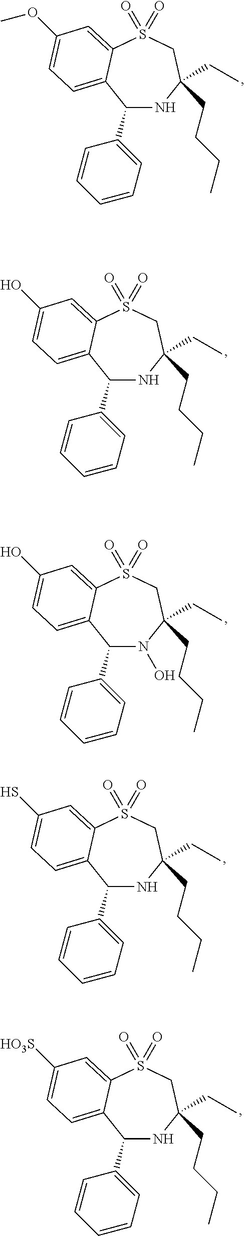

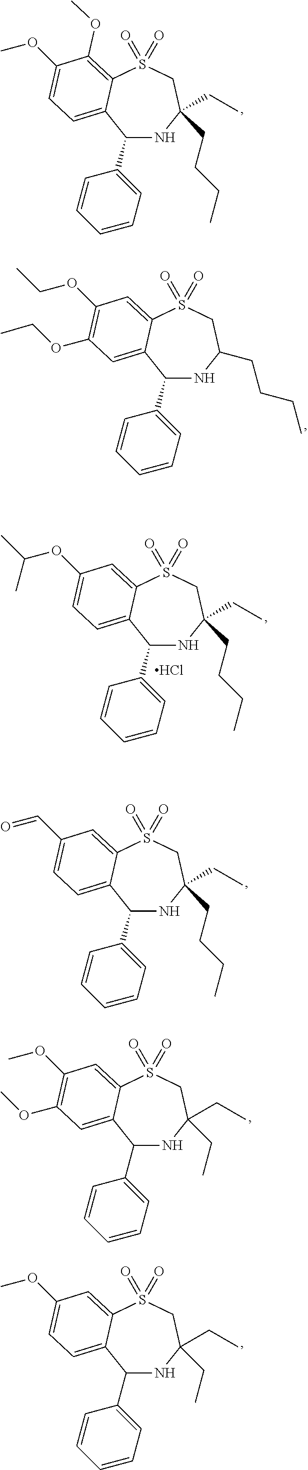

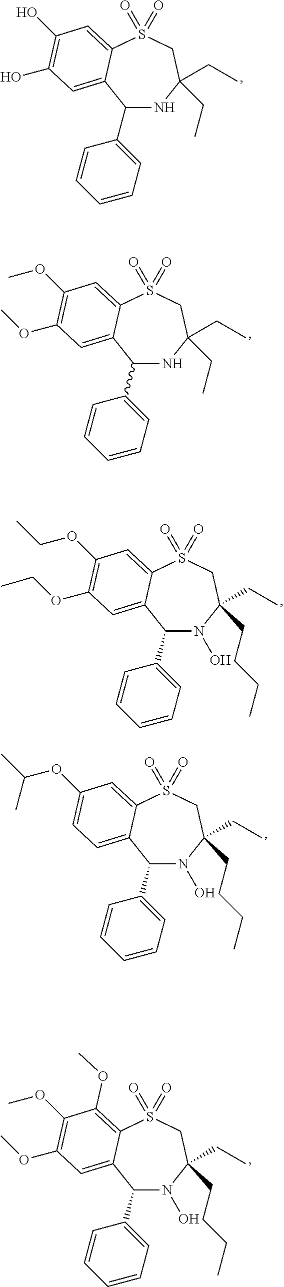

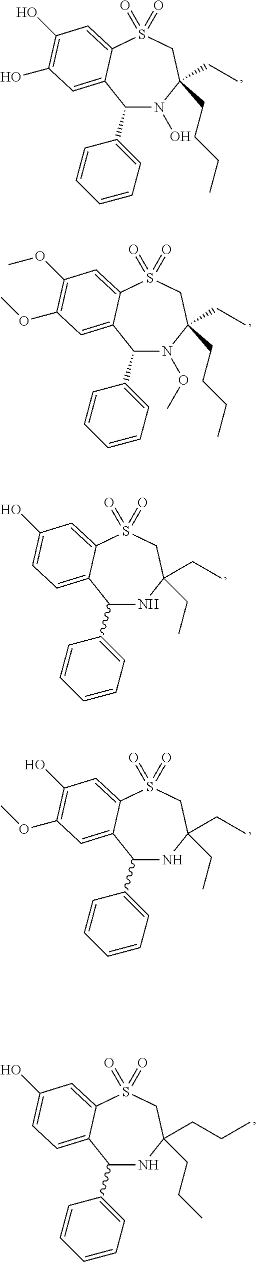

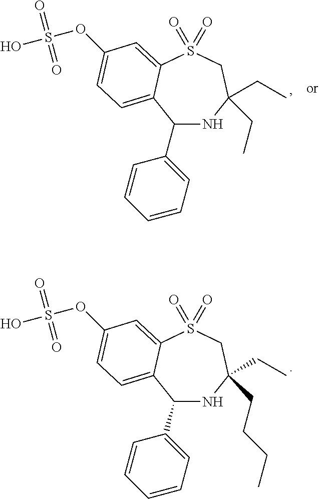 [SMILES: CCC1(CC)CS(=O)(=O)c2cc(OS(=O)(=O)O)ccc2C(c2ccccc2)N1 ; "," ; "or" ; CCCC[C@]1(CC)CS(=O)(=O)c2cc(OS(=O)(=O)O)ccc2[C@@H](c2ccccc2)N1 ; "."]

In some embodiments of the methods, the compound of Formula I is

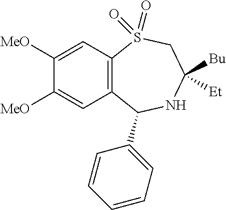

In some embodiments, an ASBTI suitable for the methods described herein is a compound of Formula II

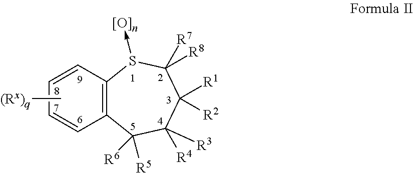

wherein:
q is an integer from 1 to 4;
n is an integer from 0 to 2;
R¹ and R² are independently selected from the group consisting of H, alkyl, alkenyl, alkynyl, haloalkyl, alkylaryl, arylalkyl, alkoxy, alkoxyalkyl, dialkylamino, alkylthio, (polyalkyl)aryl, and cycloalkyl,
wherein alkyl, alkenyl, alkynyl, haloalkyl, alkylaryl, arylalkyl, alkoxy, alkoxyalkyl, dialkylamino, alkylthio, (polyalkyl)aryl, and cycloalkyl optionally are substituted with one or more substituents selected from the group consisting of OR⁹, NR⁹R¹⁰, N⁺R⁹R¹⁰R^{w}A⁻, SR⁹, S⁺R⁹R¹⁰A⁻, P⁺R⁹R¹⁰R¹¹A⁻, S(O)R⁹, SO₂R⁹, SO₃R⁹, CO₂R⁹, CN, halogen, oxo, and CONR⁹R¹⁰,
wherein alkyl, alkenyl, alkynyl, alkylaryl, alkoxy, alkoxyalkyl, (polyalkyl)aryl, and cycloalkyl optionally have one or more carbons replaced by O, NR⁹, N⁺R⁹R¹⁰A⁻, S, SO, SO₂, S⁺R⁹A⁻, P⁺R⁹R¹⁰A⁻, or phenylene,
wherein R⁹, R¹⁰, and R^{w} are independently selected from the group consisting of H, alkyl, alkenyl, alkynyl, cycloalkyl, aryl, acyl, heterocycle, ammoniumalkyl, arylalkyl, and alkylammoniumalkyl; or
R¹ and R² taken together with the carbon to which they are attached form C₃-C₁₀ cycloalkyl;
R³ and R⁴ are independently selected from the group consisting of H, alkyl, alkenyl, alkynyl, acyloxy, aryl, heterocycle, OR⁹, NR⁹R¹⁰, SR⁹, S(O)R⁹, SO₂R⁹, and SO₃R⁹, wherein R⁹ and R¹⁰ are as defined above; or
R³ and R⁴ together ═O, ═NOR¹¹, ═S, ═NNR¹¹R¹², ═NR⁹, or ═CR¹¹R¹²
wherein R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring; R⁵ and R⁶ are independently selected from the group consisting of H, alkyl, alkenyl, alkynyl, aryl, cycloalkyl, heterocycle, quaternary heterocycle, quarternary heteroaryl, OR³⁰, SR⁹, S(O)R⁹, SO₂R⁹, SO₃R⁹, and -L_{z}-K_{z};
   wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
   wherein alkyl, alkenyl, alkynyl, aryl, cycloalkyl, heterocycle, quaternary heterocycle, and quaternary heteroaryl can be substituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂ NR¹³R¹⁴, C(O)NR¹³R¹⁴, C(O)OM, CR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻;
wherein:
   A⁻ is a pharmaceutically acceptable anion and M is a pharmaceutically acceptable cation, said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, S(O)R⁷, SO₂R⁷, SO₃R⁷, CO₂R⁷, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(O)(OR⁷) OR⁸ and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene, and R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, and quaternary heteroarylalkyl,
wherein alkyl, alkenyl, alkynyl, arylalkyl, heterocycle, and polyalkyl optionally have one or more carbons replaced by O, NR⁹, N⁺R⁹R¹⁰A⁻, S, SO, SO₂, S⁺R⁹A⁻, PR, P⁺R⁹R¹⁰A⁻, P(O)R⁹, phenylene, carbohydrate, amino acid, peptide, or polypeptide, and
R¹³, R¹⁴ and R¹⁵ are optionally substituted with one or more groups selected from the group consisting of sulfoalkyl, quaternary heterocycle, quaternary heteroaryl, OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂ NR⁹R¹⁰, PO(OR¹⁶)OR¹⁷, P⁺R⁹R¹⁰R¹¹A⁻, S⁺R⁹R¹⁰A⁻, and C(O)OM,
wherein R¹⁶ and R¹⁷ are independently selected from the substituents constituting R⁹ and M; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl; and
R⁷ and R⁸ are independently selected from the group consisting of hydrogen and alkyl; and
one or more R^{x} are independently selected from the group consisting of H, alkyl, alkenyl, alkynyl, polyalkyl, acyloxy, aryl, arylalkyl, halogen, haloalkyl, cycloalkyl, heterocycle, heteroaryl, polyether, quaternary heterocycle, quaternary heteroaryl, OR¹³, NR¹³R¹⁴, SR¹³, S(O)R¹³, S(O)₂R¹³, SO₃R¹³, S⁺R¹³R¹⁴A⁻, NR¹³OR¹⁴, NR¹³NR¹⁴R¹⁵, NO₂, CO₂R¹³, CN, OM, SO₂OM, SO₂ NR¹³R¹⁴, NR¹⁴C(O)R¹³, C(O)NR¹³R¹⁴, NR¹⁴C(O)R¹³, C(O)OM, COR¹³, OR¹⁸, S(O)ₙNR¹⁸, NR¹³R¹⁸, NR¹⁸R¹⁴, N⁺12⁹R¹¹R¹²A⁻, P⁺R⁹R¹¹R¹²A⁻, amino acid, peptide, polypeptide, and carbohydrate,
wherein alkyl, alkenyl, alkynyl, cycloalkyl, aryl, polyalkyl, heterocycle, acyloxy, arylalkyl, haloalkyl, polyether, quaternary heterocycle, and quaternary heteroaryl can be further substituted with OR⁹, NR⁹R¹⁰, N⁺R⁹R¹¹R¹²A⁻, SR⁹, S(O)R⁹, SO₂R⁹, SO₃R⁹, OXO, CO₂R⁹, CN, halogen, CONR⁹R¹⁰, SO₂OM, SO₂ NR⁹R¹⁰, PO(OR¹⁶)OR¹⁷, P R⁹R¹¹R¹² A⁻, S⁺R⁹R¹⁰A⁻, or C(O)M, and
wherein R¹⁸ is selected from the group consisting of acyl, arylalkoxycarbonyl, arylalkyl, heterocycle, heteroaryl, alkyl,
wherein acyl, arylalkoxycarbonyl, arylalkyl, heterocycle, heteroaryl, alkyl, quaternary heterocycle, and quaternary heteroaryl optionally are substituted with one or more substituents selected from the group consisting of OR⁹, NR⁹R¹⁰, N⁺R⁹R¹¹R¹²A⁻, SR⁹, S(O)R⁹, SO₂R⁹, SO₃R⁹, oxo, CO₃R⁹, CN, halogen, CONR⁹R¹⁰, SO₃R⁹, SO₂OM, SO₂ NR⁹R¹⁰, PO(OR¹⁶)OR¹⁷, and C(O)OM,
wherein in R^{x}, one or more carbons are optionally replaced by O, NR¹³, N⁺R¹³R¹⁴A⁻, S, SO, SO₂, S⁺R¹³A⁻, PR¹³, P(O)R¹³, P⁺R¹³R¹⁴A⁻, phenylene, amino acid, peptide, polypeptide, carbohydrate, polyether, or polyalkyl,
wherein in said polyalkyl, phenylene, amino acid, peptide, polypeptide, and carbohydrate, one or more carbons are optionally replaced by O, NR⁹, R⁹R¹⁰A⁻, S, SO, SO₂, S⁺R⁹A⁻, PR⁹, P⁺R⁹R¹⁰A⁻, or P(O)R⁹;
wherein quaternary heterocycle and quaternary heteroaryl are optionally substituted with one or more groups selected from the group consisting of alkyl, alkenyl, alkynyl, polyalkyl, polyether, aryl, haloalkyl, cycloalkyl, heterocycle, arylalkyl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R³, CN, OM, SO₂OM, SO₂ NR¹³R¹⁴, C(O)NR¹³R¹⁴, C(O)OM, COR¹³, P(O)R¹³R¹⁴, P⁺R¹³R¹⁴R¹⁵ A⁻, P(OR¹³)OR¹⁴, S⁺R¹³R¹⁴A⁻, and N⁺R⁹R¹¹R¹² A⁻,
provided that both R⁵ and R⁶ cannot be hydrogen or SH;
provided that when R⁵ or R⁶ is phenyl, only one of R¹ or R² is H;
   provided that when q=1 and R^{x} is styryl, anilido, or anilinocarbonyl, only one of R⁵ or R⁶ is alkyl; or a
   pharmaceutically acceptable salt, solvate, or prodrug thereof

In some embodiments of the methods, the compound of Formula II is a compound wherein
q is an integer from 1 to 4;
n is 2;
R¹ and R² are independently selected from the group consisting of H, alkyl, alkoxy, dialkylamino, and alkylthio,
wherein alkyl, alkoxy, dialkylamino, and alkylthio are optionally substituted with one or more substituents selected from the group consisting of OR⁹, NR⁹R¹⁰, SR⁹, SO₂R⁹, CO₂R⁹, CN, halogen, oxo, and CONR⁹R¹⁰;
each R⁹ and R¹⁰ are each independently selected from the group consisting of H, alkyl, cycloalkyl, aryl, acyl, heterocycle, and arylalkyl;
R³ and R⁴ are independently selected from the group consisting of H, alkyl, acyloxy, OR⁹, NR⁹R¹⁰, SR⁹, and SO₂R⁹, wherein R⁹ and R¹⁰ are as defined above;
R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R⁵ and R⁶ are independently selected from the group consisting of H, alkyl, aryl, cycloalkyl, heterocycle, and -L_{z}-K_{z};
   wherein z is 1 or 2; each L is independently a substituted or unsubstituted alkyl, a substituted or unsubstituted heteroalkyl, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
   wherein alkyl, aryl, cycloalkyl, and heterocycle can be substituted with one or more substituent groups independently selected from the group consisting of alkyl, aryl, haloalkyl, cycloalkyl, heterocycle, arylalkyl, quaternary heterocycle, quaternary heteroaryl, halogen, oxo, OR¹³, NR¹³R¹⁴, SR¹³, SO₂R¹³, NR¹³NR¹⁴R¹⁵, NO₂, CO₂R¹³, CN, OM, and CR¹³,
   wherein:
A⁻ is a pharmaceutically acceptable anion and M is a pharmaceutically acceptable cation;
R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, and quaternary heteroarylalkyl, wherein R¹³, R¹⁴ and R¹⁵ are optionally substituted with one or more groups selected from the group consisting of quaternary heterocycle, quaternary heteroaryl, OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, and CONR⁹R¹⁰; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl; and
R⁷ and R⁸ are independently selected from the group consisting of hydrogen and alkyl; and one or more R^{x} are independently selected from the group consisting of H, alkyl, acyloxy, aryl, arylalkyl, halogen, haloalkyl, cycloalkyl, heterocycle, heteroaryl, OR¹³, NR¹³R¹⁴, SR¹³, S(O)₂R¹³, NR¹³NR¹⁴R¹⁵, NO₂, CO₂R¹³, CN, SO₂ NR¹³R¹⁴, NR¹⁴C(O)R¹³, C(O)NR¹³R¹⁴, NR¹⁴C(O)R¹³, and COR¹³;
   provided that both R⁵ and R⁶ cannot be hydrogen;
   provided that when R⁵ or R⁶ is phenyl, only one of R¹ or R² is H;
   provided that when q=1 and R^{x} is styryl, anilido, or anilinocarbonyl, only one of R⁵ or R⁶ is alkyl; or a
   pharmaceutically acceptable salt, solvate, or prodrug thereof.

In some embodiments of the methods, the compound of Formula II is a compound

wherein
R⁵ and R⁶ are independently selected from the group consisting of H, aryl, heterocycle, quaternary heterocycle, and quarternary heteroaryl
wherein the aryl, heteroaryl, quaternary heterocycle and quaternary heteroaryl are optionally substituted with one or more groups selected from the group consisting of alkyl, alkenyl, alkynyl, polyalkyl, polyether, aryl, haloalkyl, cycloalkyl, heterocycle, arylalkyl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂ NR¹³R¹⁴, C(O)NR¹³R¹⁴, C(O)OM, COR¹³, P(O)R¹³R¹⁴, P⁺R¹³R¹⁴R¹⁵A⁻, P(OR¹³)OR¹⁴, S⁺R¹³R¹⁴A⁻, N⁺R⁹R¹¹R¹²A⁻ and -L_{z}-K_{z}.

In some embodiments of the methods, the compound of Formula II is a compound

wherein

### R⁵ or R⁶ is —Ar—(R^{y})ₜ

t is an integer from 0 to 5;
Ar is selected from the group consisting of phenyl, thiophenyl, pyridyl, piperazinyl, piperonyl, pyrrolyl, naphthyl, furanyl, anthracenyl, quinolinyl, isoquinolinyl, quinoxalinyl, imidazolyl, pyrazolyl, oxazolyl, isoxazolyl, pyrimidinyl, thiazolyl, triazolyl, isothiazolyl, indolyl, benzoimidazolyl, benzoxazolyl, benzothiazolyl, and benzoisothiazolyl; and
one or more R^{y} are independently selected from the group consisting of alkyl, alkenyl, alkynyl, polyalkyl, polyether, aryl, halo alkyl, cycloalkyl, heterocycle, arylalkyl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂ NR¹³R¹⁴, C(O)NR¹³R¹⁴, C(O)OM, COR¹³, P(O)R¹³R¹⁴, P⁺R¹³R¹⁴R¹⁵ A⁻, P(OR¹³)OR¹⁴, S⁺R¹³R¹⁴A⁻, N⁺R⁹R¹¹R¹²A⁻ and -L_{z}-K_{z};
wherein said alkyl, alkenyl, alkynyl, polyalkyl, polyether, aryl, haloalkyl, cycloalkyl, and heterocycle can be further substituted with one or more substituent groups selected from the group consisting of OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸A⁻, and P(O)(OR⁷)OR⁸, and or phenylene;
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene.

In some embodiments of the methods, the compound of Formula II is a compound wherein

### R⁵ or R⁶ is

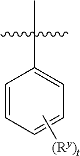

In some embodiments of the methods, the compound of Formula II is a compound wherein n is 1 or 2. In some embodiments of the methods, the compound of Formula II is a compound wherein R¹ and R² are independently H or C₁₋₇alkyl. In some embodiments of the methods, the compound of Formula II is a compound wherein each C₁₋₇alkyl is independently ethyl, n-propyl, n-butyl, or isobutyl. In some embodiments of the methods, the compound of Formula II is a compound wherein R³ and R⁴ are independently H or OR⁹. In some embodiments of the methods, compound of Formula II is a compound wherein R⁹ is H

In some embodiments of the methods, the compound of Formula II is a compound wherein one or more R^{x} are in the 7-, 8- or 9-position of the benzo ring of Formula II. In some embodiments of the methods, the compound of Formula II is a compound wherein R^{x} is in the 7-position of the benzo ring of Formula II. In some embodiments of the methods, the compound of Formula II is a compound wherein one or more R^{x} are independently selected from OR¹³ and NR¹³R¹⁴.

In some embodiments of the methods, the compound of Formula II is a compound

wherein:
q is 1 or 2;
n is 2;
R¹ and R² are each alkyl;
R³ is hydroxy;
R⁴ and R⁶ are hydrogen;
R⁵ has the formula

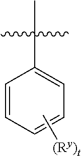

wherein
t is an integer from 0 to 5;
one or more R^{Y} are OR¹³;
R¹³ is selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, and quaternary heteroarylalkyl;
said R¹³ alkyl, alkenyl, alkynyl, arylalkyl, heterocycle, and polyalkyl groups optionally have one or more carbons replaced by O, NR⁹, N⁺R⁹R¹⁰A⁻, S, SO, SO₂, S⁺R⁹A⁻, PR⁹, P⁺R⁹R¹⁰A⁻, P(O)R⁹, phenylene, carbohydrate, amino acid, peptide, or polypeptide;
R¹³ is optionally substituted with one or more groups selected from the group consisting of sulfoalkyl, quaternary heterocycle, quaternary heteroaryl, OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂ NR⁹R¹⁰, PO(OR¹⁶)OR⁷, P⁺R⁹R¹⁰R¹¹A⁻, S⁺R⁹R¹⁰A⁻, and C(O)OM,
wherein A is a pharmaceutically acceptable anion, and M is a pharmaceutically acceptable cation,
R⁹ and R¹⁰ are independently selected from the group consisting of H, alkyl, alkenyl, alkynyl, cycloalkyl, aryl, acyl, heterocycle, ammoniumalkyl, arylalkyl, and alkylammoniumalkyl;
R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH; or
R¹¹ and R¹² together with the nitrogen or carbon atom to which they are attached form a cyclic ring; and
R¹⁶ and R¹⁷ are independently selected from the substituents constituting R⁹ and M;
R⁷ and R⁸ are hydrogen; and
one or more R^{x} are independently selected from the group consisting of alkoxy, alkylamino and dialkylamino and —W—R³¹, wherein W is O or NH and R³¹ is selected from

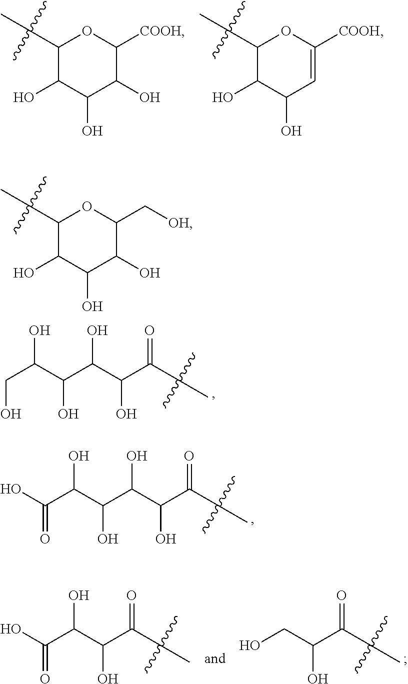

or a pharmaceutically acceptable salt, solvate, or prodrug thereof.

In some embodiments, a compound of Formula II is

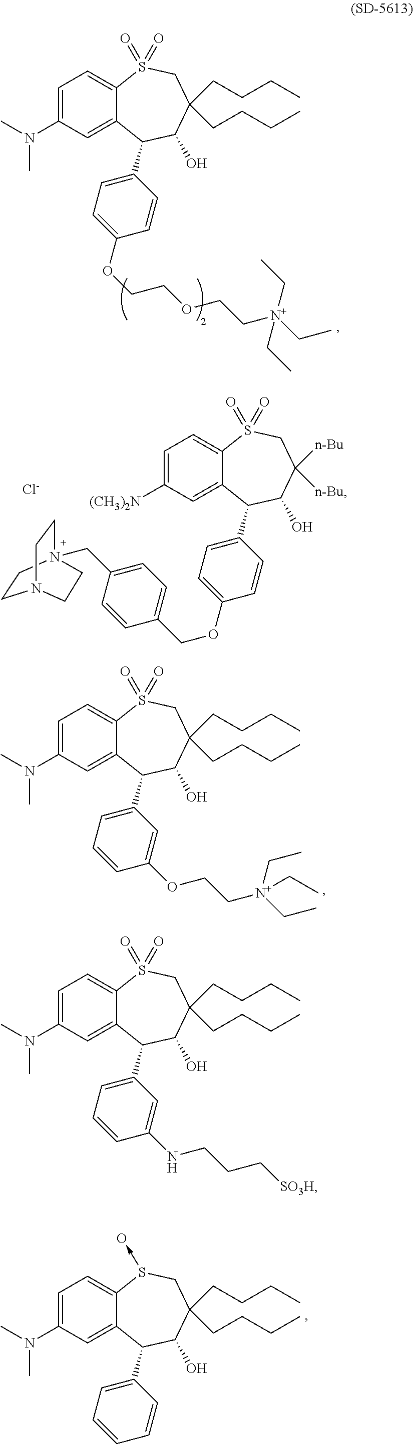

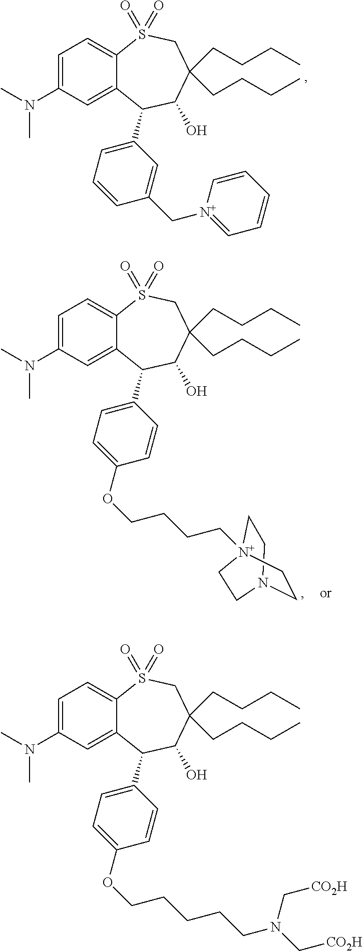

or the like.

In some embodiments of the methods, the compound of Formula II is

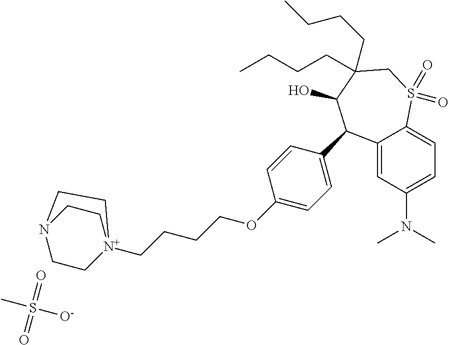

In certain embodiments, ASBTIs suitable for the methods described herein are non-systemic analogs of Compound 100C. Certain compounds provided herein are Compound 100C analogues modified or substituted to comprise a charged group. In specific embodiments, the Compound 100C analogues are modified or substituted with a charged group that is an ammonium group (e.g., a cyclic ar acyclic ammonium group). In certain embodiments, the ammonium group is a non-protic ammonium group that contains a quarternary nitrogen.

In some embodiments, a compound of Formula II is

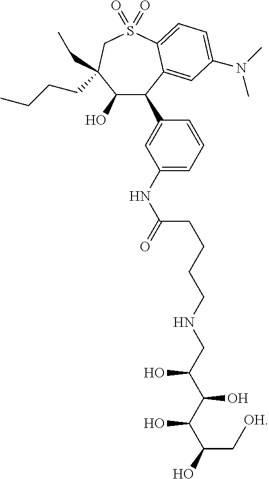 [SMILES: CCCC[C@]1(CC)CS(=O)(=O)c2ccc(N(C)C)cc2[C@@H](c2cccc(NC(=O)CCCCNC[C@H](O)[C@@H](O)[C@H](O)[C@H](O)CO)c2)[C@H]1O]

In some embodiments, a compound of Formula II is 1-[[5-[[3-[(3S,4R,5R)-3-butyl-7-(dimethylamino)-3-ethyl-2,3,4,5-tetrahydro-4-hydroxy-1,1-dioxido-1-benzothiepin-5-yl]phenyl]amino]-5-oxopentyl]amino]-1-deoxy-D-glucitol or SA HMR1741 (a.k.a. BARI-1741).

In some embodiments, a compound of Formula II is

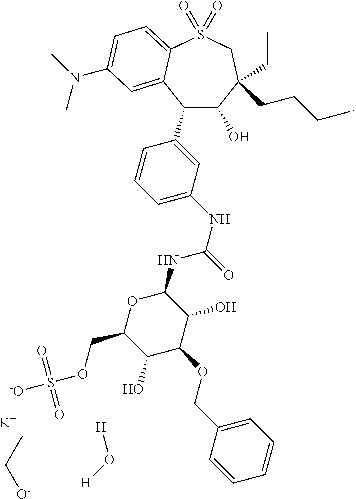

In some embodiments, a compound of Formula II is potassium((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl)sulphate ethanolate, hydrate or SAR548304B (a.k.a. SAR-548304).

In some embodiments, an ASBTI suitable for the methods described herein is a compound of Formula III:

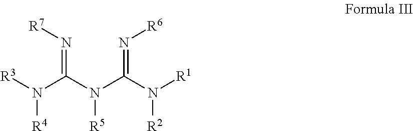

wherein:
each R¹, R² is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R¹ and R² together with the nitrogen to which they are attached form a 3-8-membered ring that is optionally substituted with R⁸;
each R³, R⁴ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
R⁵ is H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl,
each R⁶, R⁷ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R⁶ and R⁷ taken together form a bond;
each X is independently NH, S, or O;
each Y is independently NH, S, or O;
R⁸ is substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-7;
K is a moiety that prevents systemic absorption;
provided that at least one of R¹, R², R³ or R⁴ is -L-K;

or a pharmaceutically acceptable prodrug thereof.

In some embodiments of a compound of Formula III, R¹ and R³ are -L-K. In some embodiments, R¹, R² and R³ are -L-K.

In some embodiments, at least one of R¹, R², R³, R⁴, R⁵, R⁶ and R⁷ is H. In certain embodiments, R⁵, R⁶, R⁷ are H and R¹, R², R³ and R⁴ are alkyl, aryl, alkyl-aryl, or heteroalkyl. In some embodiments, R¹ and R² are H. In some embodiments, R¹, R², R⁵, R⁶ and R⁷ are H. In some embodiments, R⁶ and R⁷ together form a bond. In certain embodiments, R⁵, R⁶ and R⁷ are H, alkyl or O-alkyl.

In some embodiments, R¹ and R³ are -L-K. In some embodiments, R¹, R² and R³ are -L-K. In some embodiments, R³ and R⁴ are -L-K. In some embodiments, R¹ and R² together with the nitrogen to which they are attached form a 3-8 membered ring and the ring is substituted with -L-K. In some embodiments, R¹ or R² or R³ or R⁴ are aryl optionally substituted with -L-K. In some embodiments, R¹ or R² or R³ or R⁴ are alkyl optionally substituted with -L-K. In some embodiments, R¹ or R² or R³ or R⁴ are alky-aryl optionally substituted with -L-K. In some embodiments, R¹ or R² or R³ or R⁴ are heteroalkyl optionally substituted with -L-K.

In some embodiments, L is a C₁-C₇alkyl. In some embodiments, L is heteroalkyl. In certain embodiments, L is C₁-C₇alkyl-aryl. In some embodiments, L is C₁-C₇alkyl-aryl-C₁-C₇alkyl.

In certain embodiments, K is a non-protic charged group. In some specific embodiments, each K is a ammonium group. In some embodiments, each K is a cyclic non-protic ammonium group. In some embodiments, each K is an acyclic non-protic ammonium group.

In certain embodiments, each K is a cyclic non-protic ammonium group of structure:

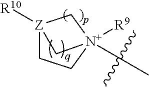

In certain embodiments, K is an acyclic non-protic ammonium group of structure:

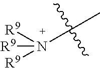

wherein p, q, R⁹, R¹⁰ and Z are as defined above. In certain embodiments, p is 1. In other embodiments, p is 2. In further embodiments, p is 3. In some embodiments, q is 0. In other embodiments, q is 1. In some other embodiments, q is 2.

The compounds further comprise 1, 2, 3 or 4 anionic counterions selected from Cl⁻, Br⁻, I⁻, R¹¹SO₃⁻, (SO₃⁻R¹¹—SO₃⁻), R¹¹CO₂⁻, (CO₂⁻R¹¹—CO₂⁻), (R¹¹)₂(P═O)O⁻ and (R¹¹)(P═O)O₂²⁻ wherein R¹¹ is as defined above. In some embodiments, the counterion is Cl⁻, Br⁻, I⁻, CH₂CO₂⁻, CH₃SO₃⁻, or C₆H₅SO₃⁻ or CO₂⁻—(CH₂)₂—CO₂⁻. In some embodiments, the compound of Formula III has one K group and one counterion. In other embodiments, the compound of Formula III has one K group, and two molecules of the compound of Formula III have one counterion. In yet other embodiments, the compound of Formula III has two K groups and two counterions. In some other embodiments, the compound of Formula III has one K group comprising two ammonium groups and two counterions.

Also described herein are compounds having the Formula IIIA:

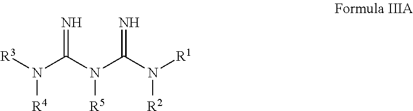

wherein:
each R¹, R² is independently H, substituted or unsubstituted alkyl, or -L-K; or R¹ and R² together with the nitrogen to which they are attached form a 3-8-membered ring that is optionally substituted with R⁸;
and R³, R⁴, R⁸, L and K are as defined above.

In some embodiments of compounds of Formula IIIA, L is Aₙ, wherein each A is substituted or unsubstituted alkyl, or substituted or unsubstituted heteroalkyl, and n is 0-7. In certain specific embodiments of the compound of Formula IIIA, R¹ is H. In some embodiments of Formula IIIA, R¹ and R² together with the nitrogen to which they are attached form a 3-8-membered ring that is optionally substituted with -L-K.

Also described herein are compounds having the Formula IIIB:

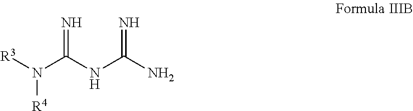

wherein:
each R³, R⁴ is independently H, substituted or unsubstituted alkyl, substituted or unsubstituted heteroalkyl, substituted or unsubstituted aryl, substituted or unsubstituted alkyl-aryl, or -L-K;
and R¹, R², L and K are as defined above.

In certain embodiments of Formula IIIB, R³ is H. In certain embodiments, R³ and R⁴ are each -L-K. In some embodiments, R³ is H and R⁴ is substituted or unsubstituted alkyl, substituted or unsubstituted heteroalkyl, substituted or unsubstituted aryl, substituted or unsubstituted alkyl-aryl containing one or two -L-K groups.

In some embodiments, an ASBTI suitable for the methods described herein is a compound of Formula IIIC

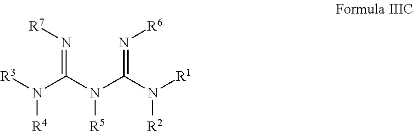

wherein:
each R¹, R² is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R¹ and R² together with the nitrogen to which they are attached form a 3-8-membered ring that is optionally substituted with R⁸;
each R³, R⁴ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
R⁵ is H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl,
each R⁶, R⁷ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R⁶ and R⁷ taken together form a bond;
each X is independently NH, S, or O;
each Y is independently NH, S, or O;
R⁸ is substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-7;
K is a moiety that prevents systemic absorption;
   or a pharmaceutically acceptable salt thereof.

In some specific embodiments of Formula I, II or III, K is selected from

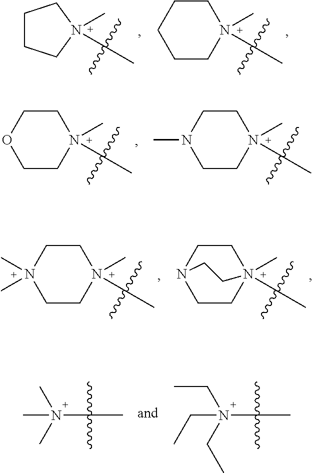

In some embodiments, an ASBTI suitable for the methods described herein is a compound of Formula IV:

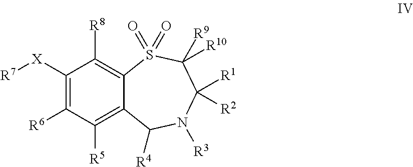

wherein

R¹ is a straight chain C₁₋₆alkyl group;

R² is a straight chain C₁₋₆alkyl group;

R³ is hydrogen or a group OR¹¹ in which R¹¹ is hydrogen, optionally substituted C₁₋₆alkyl or a C₁₋₆ alkylcarbonyl group;

R⁴ is pyridyl or an optionally substituted phenyl;

R⁵, R⁶ and R⁸ are the same or different and each is selected from: hydrogen, halogen, cyano, R¹⁵-acetylide, OR¹⁵, optionally substituted C₁₋₆alkyl, COR¹⁵, CH(OH)R¹⁵, S(O)ₙR¹⁵, P(O)(OR¹⁵)₂, OCOR¹⁵, OCF₃, OCN, SCN, NHCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂R¹⁵, OCH₂OR¹⁵, OCH═CHR¹⁵, O(CH₂CH₂O)ₙR¹⁵, O(CH₂)ₚSO₃R⁵, O(CH₂)ₚNR¹²R¹³ and O(CH₂)ₚN⁺R¹²R¹³R¹⁴ wherein

p is an integer from 1-4,

n is an integer from 0-3 and

R¹², R¹³, R¹⁴ and R¹⁵ are independently selected from hydrogen and optionally substituted C¹⁻⁶alkyl;

R⁷ is a group of the formula

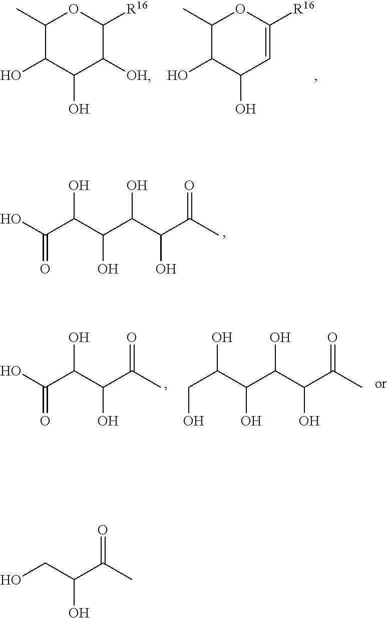

wherein the hydroxyl groups may be substituted by acetyl, benzyl, or —(C₁-C₆)-alkyl-R⁷,
wherein the alkyl group may be substituted with one or more hydroxyl groups;

R¹⁶ is —COOH, —CH₂—OH, —CH₂—O-Acetyl, —COOMe or —COOEt;

R¹⁷ is H, —OH, —NH₂, —COOH or COOR¹⁸;

R¹⁸ is (C₁-C₄)-alkyl or —NH—(C₁-C₄)-alkyl;

X is —NH— or —O—; and

R⁹ and R¹⁰ are the same or different and each is hydrogen or C₁-C₆alkyl; and salts thereof.

In some embodiments, a compound of Formula IV has the structure of Formula IVA or Formula IVB:

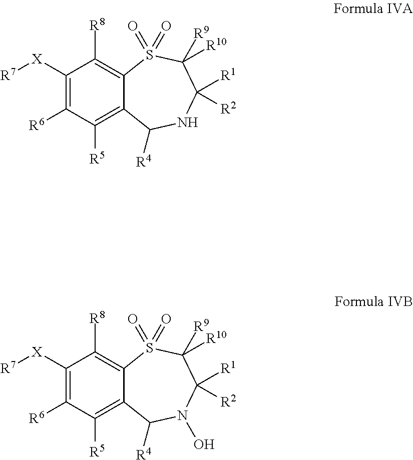

In some embodiments, a compound of Formula IV has the structure of Formula IVC:

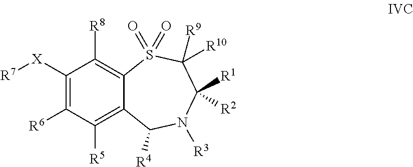

In some embodiments of Formula IV, X is O and R⁷ is selected from

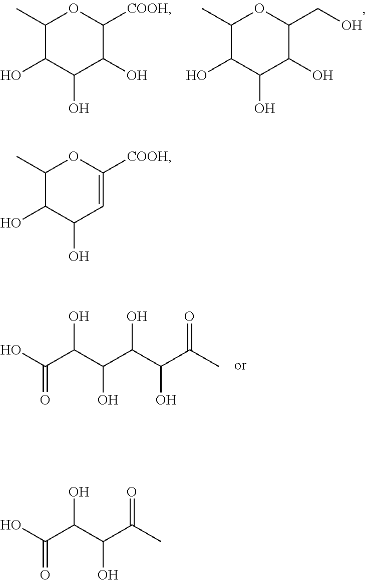

In some embodiments, a compound of Formula IV is:

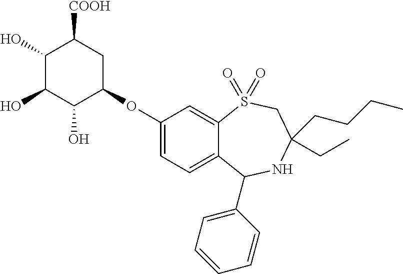 [SMILES: CCCCC1(CC)CS(=O)(=O)c2cc(O[C@@H]3C[C@H](C(=O)O)[C@@H](O)[C@H](O)[C@H]3O)ccc2C(c2ccccc2)N1]

In some embodiments, an ASBTI suitable for the methods described herein is a compound of Formula V:

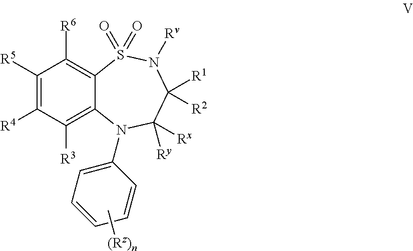

wherein:

R^{v} is selected from hydrogen or C₁₋₆alkyl;

One of R¹ and R² are selected from hydrogen or C₁₋₆alkyl and the other is selected from C₁₋₆alkyl;

R^{x} and R^{y} are independently selected from hydrogen, hydroxy, amino, mercapto, C₁₋₆alkyl, C₁₋₆ alkoxy, N—(C₁₋₆alkyl)amino, N,N—(C₁₋₆alkyl)₂amino, C₁₋₆alkylS(O)ₐ wherein a is 0 to 2;

R^{z} is selected from halo, nitr, cyano, hydroxy, amino, carboxy, carbamoyl, mercapto, sulphamoyl, C₁₋₆ alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆ alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆-alkyl)sulphamoyl and N,N—(C₁₋₆alkyl)₂sulphamoyl;

n is 0-5;

one of R⁴ and R⁵ is a group of formula (VA):

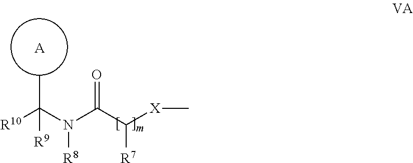

R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆ alkanoyl, C₁₋₆alkanoyloxy, N—(C₁₋₆alkyl)amino, N,N—(C₁₋₆alkyl)₂amino, C₁₋₆alkanoylamino, N—(C₁₋₆ alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl and N,N—(C₁₋₆alkyl)₂sulphamoyl;
wherein R³ and R⁶ and the other of R⁴ and R⁵ may be optionally substituted on carbon by one or more R⁷;
X is —O—, —N(R^{a})—, —S(O)_{b}— or —CH(R^{a})—;
   wherein R^{a} is hydrogen or C₁₋₆alkyl and b is 0-2;
Ring A is aryl or heteroaryl;
   wherein Ring A is optionally substituted on carbon by one or more substituents selected from R¹⁸;
R⁷ is hydrogen, C₁₋₆alkyl, carbocyclyl or heterocyclyl;
   wherein R⁷ is optionally substituted on carbon by one or more substituents selected from R¹⁹; and wherein if said heterocyclyl contains an —NH— group, that nitrogen may be optionally substituted by a group selected from R²⁰;
R⁸ is hydrogen or C₁₋₆-alkyl;
R⁹ is hydrogen or C₁₋₆alkyl;
R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkynyl, C₂₋₁₀alkynyl, C₁₋₁₀alkoxy, C₁₋₁₀ alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀ alkyl)carbamoyl, N,N—(C₁₋₁₀alkyl)₂carbamoyl, C₁₋₁₀alkylS(O)ₐ wherein a is 0 to 2, N—(C₁₋₁₀ alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²¹—(C₁₋₁₀alkylene)_{q}- or heterocyclyl-(C₁₋₁₀alkylene)ᵣ, —R²²—(C₁₋₁₀alkylene)ₛ-; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (VB):

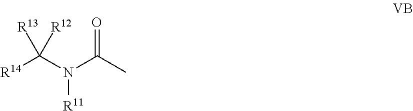

wherein:

R¹¹ is hydrogen or C₁₋₆-alkyl;

R¹² and R¹³ are independently selected from hydrogen, halo, carbamoyl, sulphamoyl, C₁₋₁₀alkyl, C₂₋₁₀alkynyl, C₂₋₁₀alkynyl, C₁₋₁₀alkanoyl, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;

R¹⁴ is selected from hydrogen, halo, carbamoyl, sulphamoyl, hydroxyaminocarbonyl, C₁₋₁₀alkyl, C₂₋₁₀alkenyl, C₂₋₁₀alkynyl, C₁₋₁₀alkanoyl, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷—(C₁₋₁₀alkylene)_{q}- or heterocyclyl-(C₁₋₁₀alkylene)ᵣ-R²⁸—(C₁₋₁₀alkylene)ₛ-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (VC):

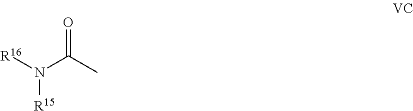

R¹⁵ is hydrogen or C₁₋₆alkyl; and R¹⁶ is hydrogen or C₁₋₆alkyl; wherein R¹⁶ may be optionally substituted on carbon by one or more groups selected from R³¹;

or R¹⁵ and R¹⁶ together with the nitrogen to which they are attached form a heterocyclyl; wherein said heterocyclyl may be optionally substituted on carbon by one or more R³⁷; and wherein if said heterocyclyl contains an —NH— group, that nitrogen may be optionally substituted by a group selected from R³⁸;

m is 1-3; wherein the values of R⁷ may be the same or different;

R¹⁷, R¹⁸, R¹⁹, R²³, R²⁵, R²⁹, R³¹ and R³⁷ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀ alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀ alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)ᵣ-R³³—(C₁₋₁₀alkylene)ₛ-; wherein R¹⁷, R¹⁸, R¹⁹, R²³, R²⁵, R²⁹, R³¹ and R³⁷ may be independently optionally substituted on carbon by one or more R³⁴; and wherein if said heterocyclyl contains an —NH— group, that nitrogen may be optionally substituted by a group selected from R³⁵;

R²¹, R²², R²⁷, R²⁸, R³² or R³³ are independently selected from —O—O—, —NR³⁶—, —S(O)ₓ—, —NR³⁶C(O)NR³⁶—, —NR³⁶C(S)NR³⁶—, —OC(O)N═C—, —NR³⁶C(O)—or —C(O)NR³⁶—; wherein R³⁶ is selected from hydrogen or C₁₋₁₆alkyl, and x is 0-2;

p, q, r and s are independently selected from 0-2;

R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, allyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;

R²⁰, R²⁴, R²⁶, R³⁰, R³⁵ and R³⁸ are independently selected from C₁₋₆alkyl, C₁₋₆alkanoyl, C₁₋₆ alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; and

wherein a “heteroaryl” is a totally unsaturated, mono or bicyclic ring containing 3-12 atoms of which at least one atom is chosen from nitrogen, sulphur and oxygen, which heteroaryl may, unless otherwise specified, be carbon or nitrogen linked;

wherein a “heterocyclyl” is a saturated, partially saturated or unsaturated, mono or bicyclic ring containing 3-12 atoms of which at least one atom is chosen from nitrogen, sulphur and oxygen, which heterocyclyl may, unless otherwise specified, be carbon or nitrogen linked, wherein a —CH₂— group can optionally be replaced by a —C(O)— group, and a ring sulphur atom may be optionally oxidised to form an S-oxide; and

wherein a “carbocyclyl” is a saturated, partially saturated or unsaturated, mono or bicyclic carbon ring that contains 3-12 atoms; wherein a —CH₂— group can optionally be replaced by a —C(O) group;

or a pharmaceutically acceptable salt or in vivo hydrolysable ester or amide formed on an available carboxy or hydroxy group thereof.

In some embodiments, an ASBTI suitable for the methods described herein is a compound of Formula VI:

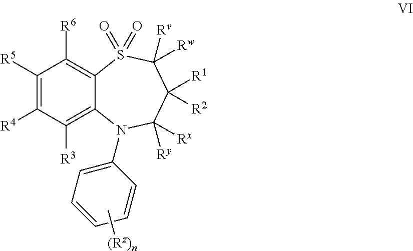

wherein:

R^{v} and R^{w} are independently selected from hydrogen or C₁₋₆alkyl;

one of R¹ and R² is selected from hydrogen or C₁₋₆alkyl and the other is selected from C₁₋₆alkyl;

R^{x} and R^{y} are independently selected from hydrogen or C₁₋₆alkyl, or one of R^{x} and R^{y} is hydrogen or C₁₋₆alkyl and the other is hydroxy or C₁₋₆alkoxy;

R^{z} is selected from halo, nitro, cyano, hydroxy, amino, carboxy, carbamoyl, mercapto, sulphamoyl, C₁₋₆alkyl, C₂₋₆alkenyl, C₂₋₆ alkynyl, C₁₋₆alkoxy, C₁₋₁₆ alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl and N,N—(C₁₋₆alkyl)₂sulphamoyl;

n is 0-5;

one of R⁴ and R⁵ is a group of formula (VIA):

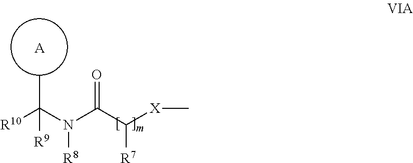

R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆ alkoxy, C₁₋₆ alkanoyl, C₁₋₆alkanoyloxy, N—(C₁₋₆alkyl)amino, N,N—(C₁₋₆alkyl)₂amino, C₁₋₆alkanoylamino, N—(C₁₋₆ alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl and N,N—(C₁₋₁₆alkyl)₂sulphamoyl; wherein R³ and R⁶ and the other of R⁴ and R⁵ may be optionally substituted on carbon by one or more R¹⁷;

X is —O—, —N(R^{a})—, —S(O)_{b}— or —CH(R^{a})—; wherein R^{a} is hydrogen or C₁₋₆alkyl and b is 0-2;

Ring A is aryl or heteroaryl; wherein Ring A is optionally substituted on carbon by one or more substituents selected from R¹⁸;

R⁷ is hydrogen, C₁₋₁₆alkyl, carbocyclyl or heterocyclyl; wherein R⁷ is optionally substituted on carbon by one or more substituents selected from R¹⁹; and wherein if said heterocyclyl contains an —NH— group, that nitrogen may be optionally substituted by a group selected from R²⁰;

R⁸ is hydrogen or C₁₋₆alkyl;

R⁹ is hydrogen or C₁₋₆alkyl;

R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene), —R²¹—(C₁₋₁₀alkylene)_{q}- or heterocyclyl-(C₁₋₁₀alkylene)ᵣ-R²²—(C₁₋₁₀alkylene)-; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (VIB):

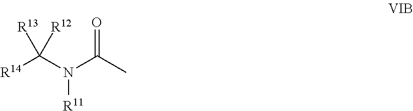

wherein:

R¹¹ is hydrogen or C₁₋₆alkyl;

R¹² and R¹³ are independently selected from hydrogen, halo, nitro, cyano, hydroxy, amino, carbamoyl, mercapto, sulphamoyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;

R¹⁴ is selected from hydrogen, halo, nitro, cyano, hydroxy, amino, carbamoyl, mercapto, sulphamoyl, hydroxyaminocarbonyl, Cl₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷—(C₁₋₁₀alkylene)_{q}- or heterocyclyl-(C₁₋₁₀alkylene)ᵣ-R²⁸—(C₁₋₁₀alkylene)-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (VIC):

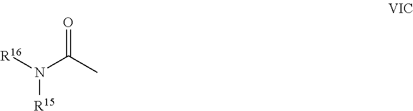

R¹⁵ is hydrogen or C₁₋₆alkyl;

R¹⁶ is hydrogen or C₁₋₆alkyl; wherein R¹⁶ may be optionally substituted on carbon by one or more groups selected from R³¹;

n is 1-3; wherein the values of R⁷ may be the same or different;

R¹⁷, R¹⁸, R¹⁹, R²³, R²⁵, R²⁹ or R³¹ are independently selected from halo, nitro, cyano, hydroxy, amino, carbamoyl, mercapto, sulphamoyl, hydroxyaminocarbonyl, amidino, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, (C₁₋₁₀alkyl)₃ silyl, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)a wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)ᵣ-R³³—(C₁₋₁₀alkylene)-; wherein R¹⁷, R¹⁸, R¹⁹, R²³, R²⁵, R²⁹ or R³¹ may be independently optionally substituted on carbon by one or more R³⁴; and wherein if said heterocyclyl contains an —NH— group, that nitrogen may be optionally substituted by a group selected from R³⁵;

R²¹, R²², R²⁷, R²⁸, R³² or R³³ are independently selected from —O—, —NR³⁶—, —S(O)ₓ, NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;

p, q, r and s are independently selected from 0-2;

R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, allyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;

R²⁰, R²⁴, R²⁶, R³⁰ or R³⁵ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

or a pharmaceutically acceptable salt, solvate or solvate of such a salt, or an in vivo hydrolysable ester formed on an available carboxy or hydroxy thereof, or an in vivo hydrolysable amide formed on an available carboxy thereof.

In some embodiments, a compound of Formula VI has the structure of Formula VID:

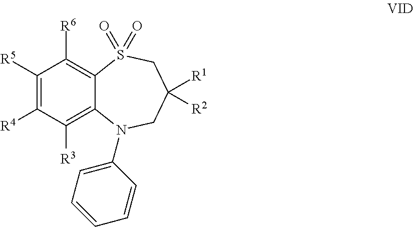

wherein:

R¹ and R² are independently selected from C₁₋₆alkyl; one of R⁴ and R⁵ is a group of formula (VIE):

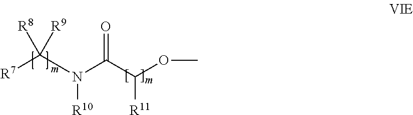

R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄ alkynyl, C₁₋₄alkoxy, C₁₋₄ alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄ alkyl)sulphamoyl and N,N—(C₁₋₁₄alkyl)₂sulphamoyl; wherein R³ and R⁶ and the other of R⁴ and R⁵ may be optionally substituted on carbon by one or more R⁴;

R⁷ is carboxy, sulpho, sulphino, phosphono, —P(O)(OR^{a})(OR^{b}), P(O)(OH)(ORₐ), —P(O)(OH)(R^{a}) or P(O)(OR^{a})(R^{b}), wherein R^{a} and R^{b} are independently selected from C₁₋₆alkyl; or R⁷ is a group of formula (VIF):

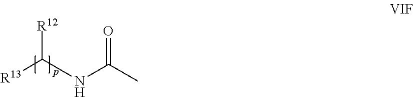

R⁸ and R⁹ are independently hydrogen, C₁₋₄alkyl or a saturated cyclic group, or R⁸ and R⁹ together form C₂₋₆alkylene; wherein R⁸ and R⁹ or R⁸ and R⁹ together may be independently optionally substituted on carbon by one or more substituents selected from R¹⁵; and wherein if said saturated cyclic group contains an —NH— moiety, that nitrogen may be optionally substituted by one or more R²⁰;

R¹⁰ is hydrogen or C₁₋₄alkyl; wherein R¹⁰ is optionally substituted on carbon by one or more substituents selected from R²⁴;

R¹¹ is hydrogen, C¹⁻⁴alkyl, carbocyclyl or heterocyclyl; wherein R¹ is optionally substituted on carbon by one or more substituents selected from R¹⁶; and wherein if said heterocyclyl contains an —NH— moiety, that nitrogen may be optionally substituted by one or more R²¹;

R¹² is hydrogen or C₁₋₄alkyl, carbocyclyl or heterocyclyl; wherein R¹² optionally substituted on carbon by one or more substituents selected from R¹⁷; and wherein if said heterocyclyl contains an —NH— moiety, that nitrogen may be optionally substituted by one or more R²²;

R¹³ is carboxy, sulpho, sulphino, phosphono, —P(O)(OR^{c})(OR^{d}), —P(O)(OH)(OR), —P(O)(OH)(R^{e)} or —P(O)(OR^{c})(R^{d}) wherein R^{c} and R^{d} are independently selected from C₁₋₆alkyl;

m is 1-3; wherein the values of R⁸ and R⁹ may be the same or different;

n is 1-3; wherein the values of R¹¹ may be the same or different;

p is 1-3; wherein the values of R¹² may be the same or different;

R¹⁴ and R¹⁶ are independently selected from halo, nitro, cyano, hydroxy, amino, carboxy, carbamoyl, mercapto, sulphamoyl, C₁₋₄alkyl, C₂₋₄ alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl and N,N—(C₁₋₄alkyl)₂sulphamoyl; wherein R¹⁴ and R¹⁶ may be independently optionally substituted on carbon by one or more R¹⁸;

R¹⁵ and R¹⁷ are independently selected from halo, nitro, cyano, hydroxy, amino, carboxy, carbamoyl, mercapto, sulphamoyl, C₁₋₄alkyl, C₂₋₄ alkenyl, C₂₋₄alkynyl, C₁₋₄alkoxy, C₁₋₄alkanoyl, C₁₋₄ alkanoyloxy, N—(C₁₋₄alkyl)amino, N,N—(C₁₋₄alkyl)₂amino, C₁₋₄ alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl and N,N—(C₁₋₄ alkyl)₂sulphamoyl, carbocyclyl, heterocyclyl, sulpho, sulphino, amidino, phosphono, —P(O)(OR^{e})(OR^{f}), —P(O)(OH)(OR^{e}), —P(O)(OH)(R^{e}) or —P(O)(OR^{e})(R^{f}), wherein R^{e} and R^{f} are independently selected from C₁₋₆ alkyl; wherein R¹⁵ and R¹⁷ may be independently optionally substituted on carbon by one or more R¹⁹; and wherein if said heterocyclyl contains an —NH— moiety, that nitrogen may be optionally substituted by one or more R²³;

R¹⁸, R¹⁹ and R²⁵ are independently selected from halo, hydroxy, cyano, carbamoyl, ureido amino nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;

R²⁰, R²¹, R²², R²³ and R²⁶ are independently C₁₋₄alkyl, C₁₋₄alkanoyl, C₁₋₄alkylsulphonyl, sulphamoyl, N—(C₁₋₄alkyl)sulphamoyl, N,N—(C₁₋₄alkyl)₂sulphamoyl, C₁₋₄alkoxycarbonyl, carbamoyl, N—(C₁₋₄alkyl)carbamoyl, N,N—(C₁₋₄alkyl)₂carbamoyl, benzyl, phenethyl, benzoyl, phenylsulphonyl and phenyl;

R²⁴ is selected from halo, nitro, cyano, hydroxy, amino, carboxy, carbamoyl, mercapto, sulphamoyl, C₁₋₄alkyl, C₂₋₄ alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl and N,N—(C₁₋₄alkyl)₂sulphamoyl, carbocyclyl, heterocyclyl; wherein R²⁴ may be independently optionally substituted on carbon by one or more R²⁵; and wherein if said heterocyclyl contains an —NH— moiety, that nitrogen may be optionally substituted by one or more R²⁶;

wherein any saturated cyclic group is a totally or partially saturated, mono or bicyclic ring containing 3-12 atoms of which 0-4 atoms are chosen from nitrogen, sulphur or oxygen, which may be carbon or nitrogen linked;

wherein any heterocyclyl is a saturated, partially saturated or unsaturated, mono or bicyclic ring containing 3-12 atoms of which at least one atom is chosen from nitrogen, sulphur or oxygen, which may be carbon or nitrogen linked, wherein a —CH₂— group can optionally be replaced by a —C(O)— or a ring sulphur atom may be optionally oxidised to form the S-oxides; and

wherein any carbocyclyl is a saturated, partially saturated or unsaturated, mono or bicyclic carbon ring that contains 3-12 atoms, wherein a —CH₂— group can optionally be replaced by a —C(O)—;

or a pharmaceutically acceptable salt thereof.

In some embodiments, any compound described herein is covalently conjugated to a bile acid using any suitable method. In some embodiments, compounds described herein are covalently bonded to a cyclodextrin or a biodegradable polymer (e.g., a polysaccharide).

In certain embodiments compounds described herein are not systemically absorbed. Moreover, provided herein are compounds that inhibit bile salt recycling in the gastrointestinal tract of an individual. In some embodiments, compounds described herein, may not be transported from the gut lumen and/or do not interact with ASBT. In some embodiments, compounds described herein, do not affect, or minimally affect, fat digestion and/or absorption. In certain embodiments, the administration of a therapeutically effective amount of any compound described herein does not result in gastrointestinal disturbance or lactic acidosis in an individual. In certain embodiments, compounds described herein are administered orally. In some embodiments, an ASBTI is released in the distal ileum. An ASBTI compatible with the methods described herein may be a direct inhibitor, an allosteric inhibitor, or a partial inhibitor of the Apical Sodium-dependent Bile acid Transporter.

In certain embodiments, compounds that inhibit ASBT or any recuperative bile acid transporters are compounds that are described in EP1810689, U.S. Pat. Nos. 6,458,851, 7,413,536, 7,514,421, US Appl. Publication Nos. 2002/0147184, 2003/0119809, 2003/0149010, 2004/0014806, 2004/0092500, 2004/0180861, 2004/0180860, 2005/0031651, 2005/0101611, 2005/0124557, 2006/0069080, 2006/0083790, 2006/0199797, 2006/0241121, 2007/0065428, 2007/0066644, 2007/0161578, 2007/0197628, 2007/0203183, 2007/0254952, 2008/0070888, 2008/0070892, 2008/0070889, 2008/0070984, 2008/0089858, 2008/0096921, 2008/0161400, 2008/0167356, 2008/0194598, 2008/0255202, 2008/0261990, 2012/0114588, WO 2002/50027, WO2005/046797, WO2006/017257, WO2006/105913, WO2006/105912, WO2006/116499, WO2006/117076, WO2006/121861, WO2006/122186, WO2006/124713, WO2007/050628, WO2007/101531, WO2007/134862, WO2007/140934, WO2007/140894, WO2008/028590, WO2008/033431, WO2008/033464, WO2008/031501, WO2008/031500, WO2008/033465, WO2008/034534, WO2008/039829, WO2008/064788, WO2008/064789, WO2008/088836, WO2008/104306, WO2008/124505, WO2008/130616, WO12064266, WO12064267, and WO12064268; the compounds described therein that inhibit recuperative bile acid transport are hereby incorporated herein by reference.

In certain embodiments, compounds that inhibit ASBT or any recuperative bile acid transporters are compounds described in WO93/16055, WO94/18183, WO94/18184, WO96/05188, WO96/08484, WO96/16051, WO97/33882, WO98/38182, WO99/35135, WO98/40375, WO99/64409, WO99/64410, WO00/01687, WO00/47568, WO00/61568, DE 19825804, WO00/38725, WO00/38726, WO00/38727 (including those compounds with a 2,3,4,5-tetrahydro-1-benzothiepine 1,1-dioxide structure), WO00/38728, WO00062810, WO01/66533, WOO02/50051, WOO2032428, WO03106482, WO03091232, WO03061663, WO03022830, WOO04076430, WO4089350, WO04006899, WO4020421, EP0864582 (e.g. (3R,5R)-3-butyl-3-ethyl-1,1-dioxido-5-Phenyl-2,3,4,5-tetrahydro-1,4-benzo-thiazepin-8-yl(β-D-glucopyranosiduronic acid, WO94/24087, WO98/07749, WO98/56757, WO99/32478, WO99/35135, WO00/20392, WO00/20393, WO00/20410, WO00/20437, WO01/34570, WO00/35889, WO01/68637, WO01/68096, WOO02/08211, WO03/020710, WOO03/022825, WOO03/022830, WOO03/022286, JP10072371, U.S. Pat. Nos. 5,910,494; 5,723,458; 5,817,652; 5,663,165; 5,998,400; 6,465,451, 5,994,391; 6,107,494; 6,387,924; 6,784,201; 6,875,877; 6,740,663; 6,852,753; 5,070,103, 6,114,322, 6,020,330, 7,125,864, 7,132,416, 7,179,792, 7,192,945, 7,192,946, 7,192,947, 7,226,943, 7,312,208, 7,803,792, 8,067,584, EP251315, EP417725, EP489-423, EP549967, EP573848, EP624593, EP624594, EP624595, EP869121, EP1070703, WOO04/005247, compounds disclosed as having IBAT activity in Drugs of the Future, 24, 425-430 (1999), Journal of Medicinal Chemistry, 48, 5837-5852, (2005) and Current Medicinal Chemistry, 13, 997-1016, (2006); the compounds described therein that inhibit recuperative bile acid transport are hereby incorporated herein by reference.

In some embodiments, compounds that inhibit ASBT or any recuperative bile acid transporter are benzothiepines, benzothiazepines (including 1,2-benzothiazepines; 1,4-benzothiazepines; 1,5-benzothiazepines; and/or 1,2,5-benzothiadiazepines). In some embodiments, compounds that inhibit ASBT or any recuperative bile acid transporter include and are not limited to S-8921 (disclosed in EP597107, WO 93/08155), 264W94 (GSK) disclosed in WO 96/05188; SC-435 (1-[4-[4-[(4R,5R)-3,3-dibutyl-7-(dimethylamino)-2,3,4,5-tetrahydro-4-hydroxy-1,1-dioxido-benzothiepin-5-yl]phenoxy]butyl]-4-aza-1-azoniabicyclo[2.2.2]octane methanesulfonate salt), SC-635 (Searle); 2164U90 (3-butyl-3-ethyl-2,3,4,5-tetrahydro-5-phenyl-1,4-benzothiazepine 1,1-dioxide); BARI-1741 (Aventis SA), AZD 7508 (Astra Zeneca); barixibat (11-(D-gluconamido)-N-{2-[(1S,2R,3S)-3-hydroxy-3-phenyl-2-(2-pyridyl)-1-(2-pyridylamino)propyl]phenyl}undecanamide) or the like, or combinations thereof. In some embodiments, an ASBTI is:

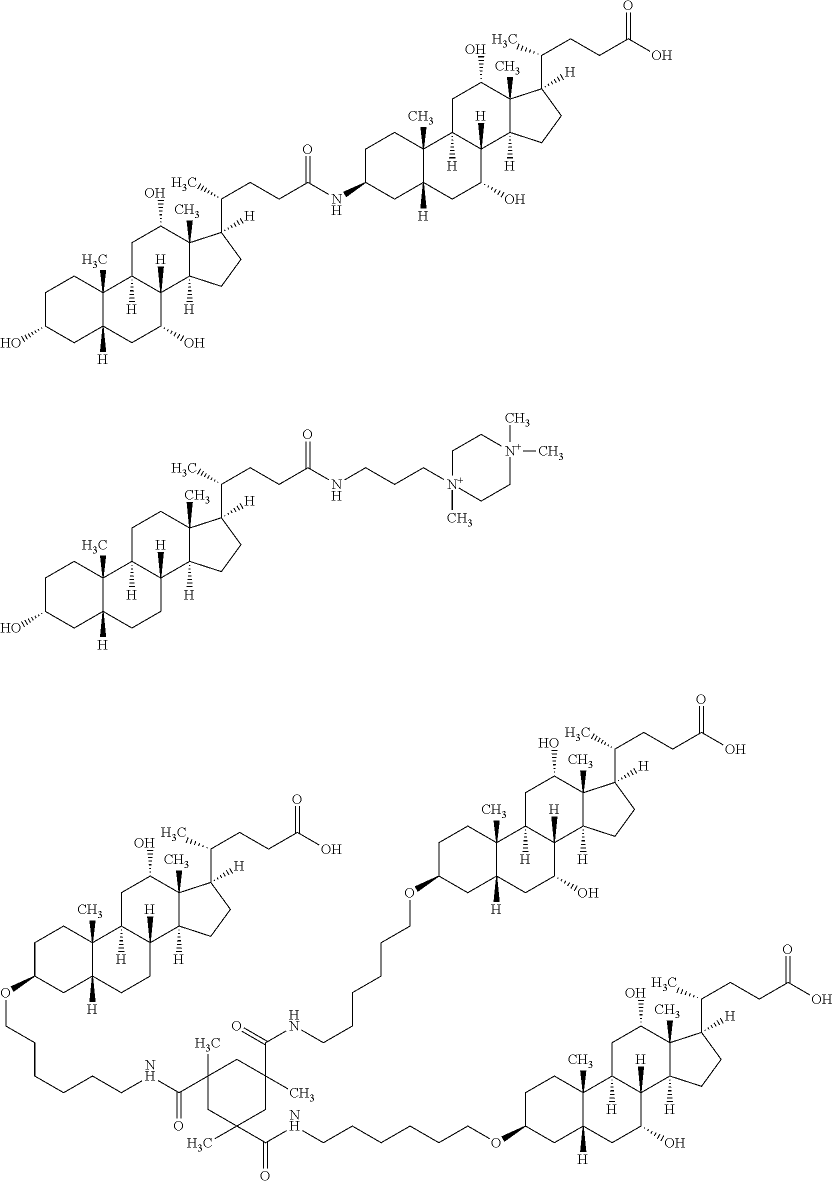

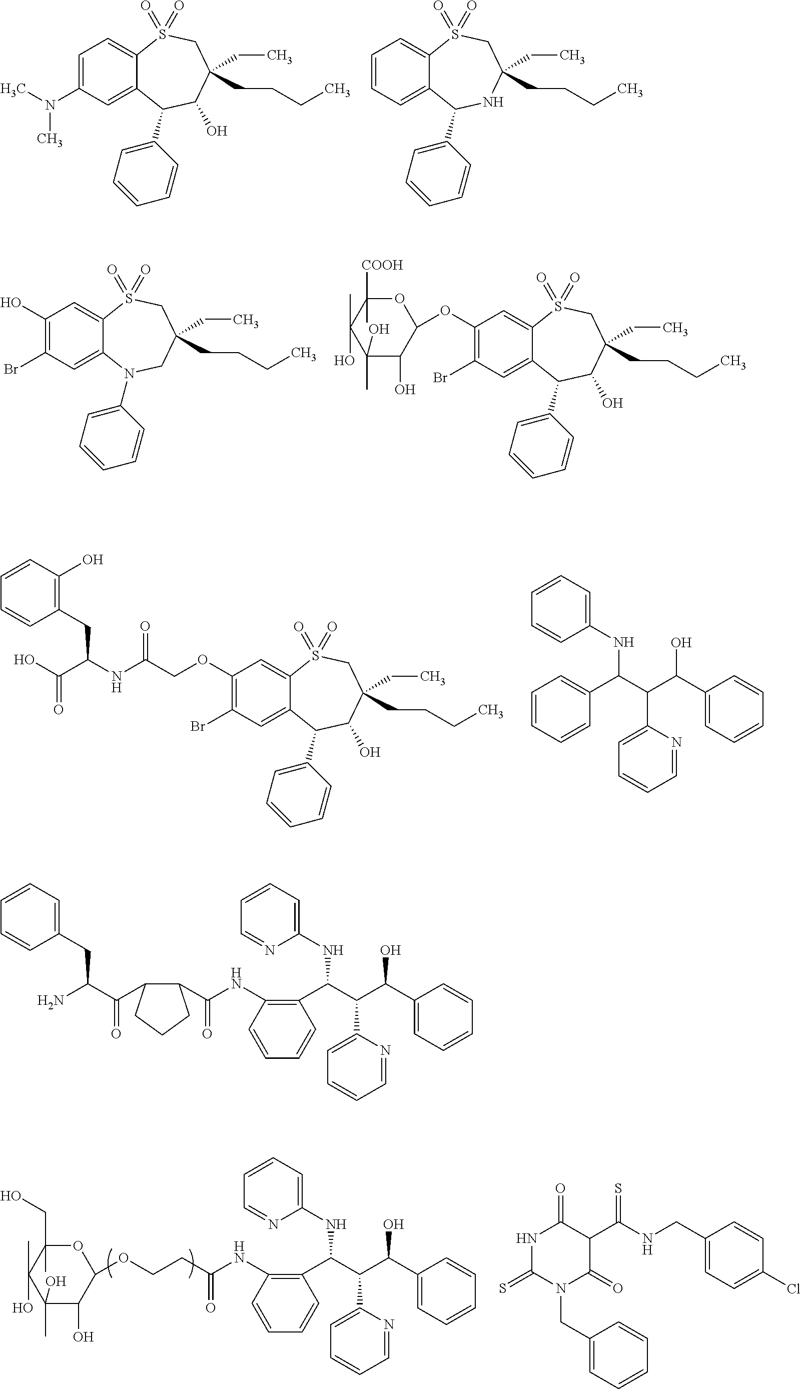

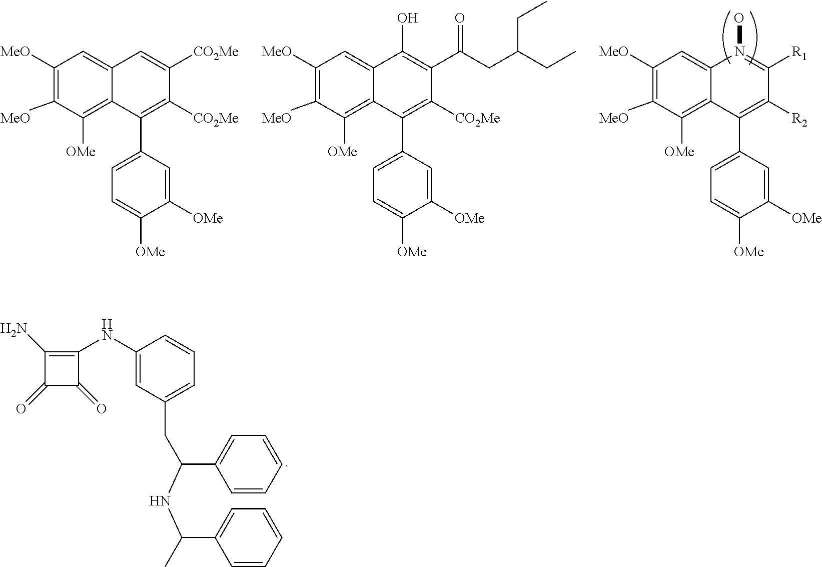

In some embodiments, an enteroendocrine peptide secretion enhancing agent, bile acid, or bile acid mimic used in any composition or method described herein is a compound of Formula X:

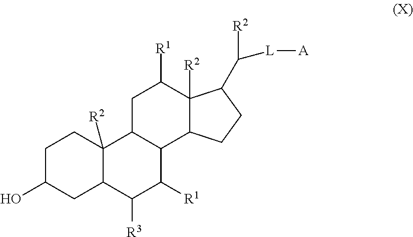

In certain embodiments, each R1 is independently H, OH, O-lower alkyl (e.g., OCH3, or OEt). In some embodiments, each R1 is independently H, OH, lower (e.g., C1-C6 or C1-C3) alkyl, or lower (e.g., C1-C6 or C1-C3) heteroalkyl. In certain embodiments, L is a substituted or unsubstituted alkyl or substituted or unsubstituted heteroalkyl. In some embodiments, R2 is H, OH, lower alkyl, or lower heteroalkyl (e.g., OMe). In certain embodiments, R3 is H, OH, O-lower alkyl, lower alkyl, or lower heteroalkyl (e.g., OMe). In some embodiments, A is COOR4, S(O)nR4, or OR5. In certain embodiments, R4 is H, an anion, a pharmaceutically acceptable cation (e.g., an alkali metal cation, alkaline earth metal cation, or any other pharmaceutically acceptable cation) substituted or unsubstituted alkyl, substituted or unsubstituted heteroalkyl, substituted or unsubstituted aryl, substituted or unsubstituted heteroaryl, an amino acid, or the like; and n is 1-3. Each R5 is independently selected from lower alkyl and H.

In specific embodiments, L is unsubstituted branched or straight chain alkyl. In more specific embodiments, L is unsubstituted branched or straight chain lower alkyl. In some embodiments, L is (CR52)ₘ—CONR5-(CR52)p. Each m is 1-6 and n is 1-6. In specific embodiments, m is 2 and n is 1. In other specific embodiments, m is 2 and n is 2. In certain embodiments, A is COOH or COO—. In some embodiments, A is SO3H or SO3—.

In specific embodiments, the compound of Formula X has a structure represented by Formula (Xa):

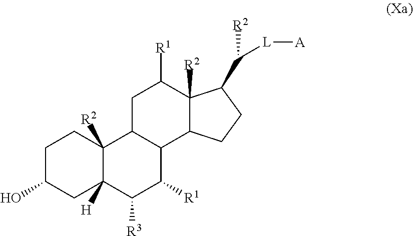

In some embodiments, bile acid mimics include, by way of non-limiting example, 6-methyl-2-oxo-4-thiophen-2-yl-1,2,3,4-tetrahydro-phyrimidine-5-carboxylic acid benzyl ester (or TGR5-binding analogs thereof), oleanolic acid (or other free fatty acids), or the like.

In certain embodiments, compounds described herein have one or more chiral centers. As such, all stereoisomers are envisioned herein. In various embodiments, compounds described herein are present in optically active or racemic forms. It is to be understood that the compounds of the present invention encompasses racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, compounds described herein contains one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as described herein are modified by the use of appropriate reagents and conditions, for the introduction of the various moieties found in the formulae as provided herein. As a guide the following synthetic methods are utilized.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linkages. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in then presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

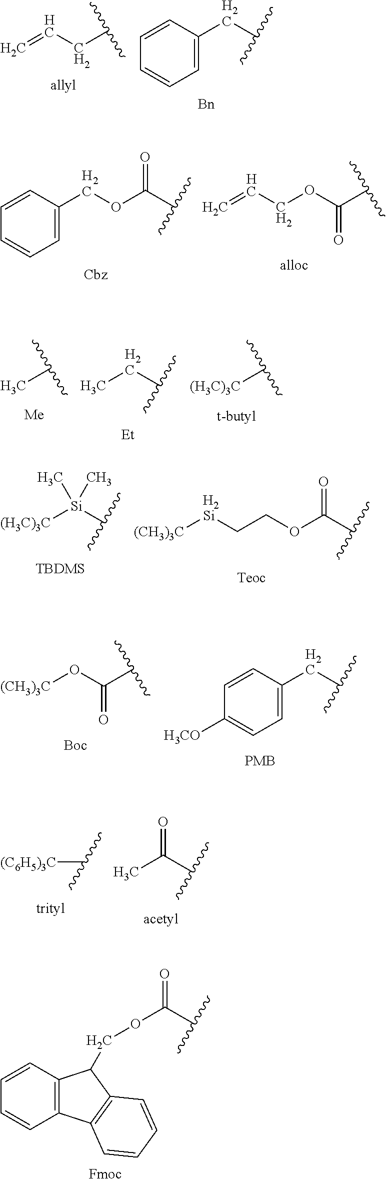

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

In some embodiments, ASBTIs described herein are synthesized as described in, for example, WO 96/05188, U.S. Pat. Nos. 5,994,391; 7,238,684; 6,906,058; 6,020,330; and 6,114,322. In some embodiments, ASBTIs described herein are synthesized starting from compounds that are available from commercial sources or that are prepared using procedures outlined herein. In some embodiments, compounds described herein are prepared according to the process set forth in Scheme 1:

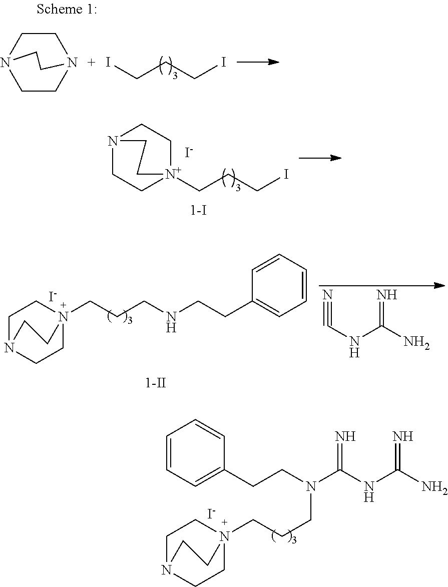

In certain embodiments, the synthesis begins with a reaction of 1,4-diazabicyclo[2.2.2]octane with 4-iodo-1-chloro butane to provide a compound of structure 1-I. Such compounds are prepared in any suitable manner, e.g., as set forth in Tremont, S. J. et. al., J. Med. Chem. 2005, 48, 5837-5852. The compound of structure 1-I is then subjected to a reaction with phenethylamine to provide a compound of structure 1-II. The compound of structure 1-II is then allowed to react with dicyanodiamide to provide a compound of Formula I.

In some embodiments, a first compound of Formula III is subjected to a further reaction to provide a second compound of Formula III as shown in Scheme 2 below.

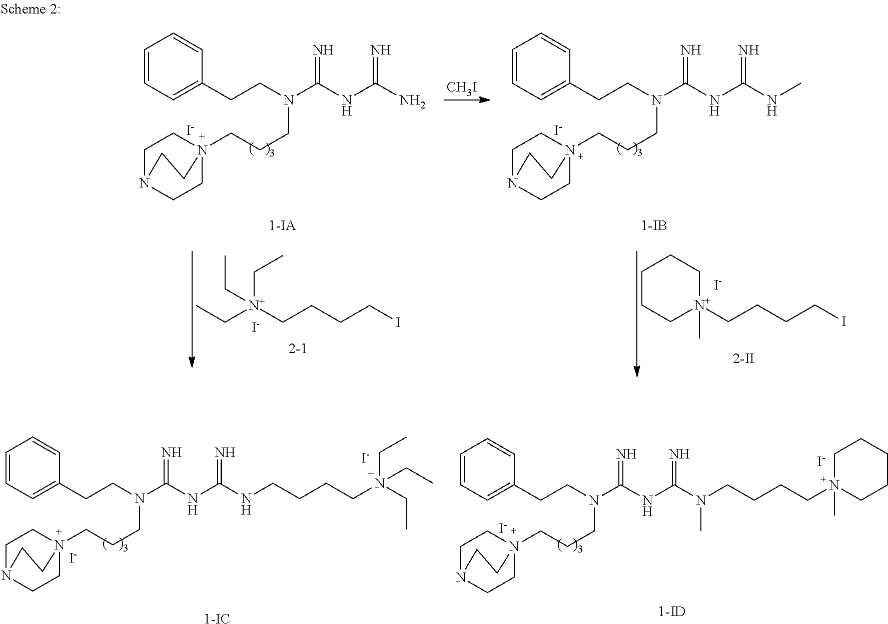

A first compound of Formula III, 1-IA, is alkylated with iodomethane to provide a second compound of Formula III, 1-IB. Alkylation of 1-IB with a compound of structure 2-II provides a further compound of Formula III, IC. In an alternative embodiment, a first compound of Formula III, 1-IA, is alkylated with a compound of structure 2-I to provide a second compound of Formula III, 1-IC.

### General Definitions

The term “bile acid,” as used herein, includes steroid acids (and/or the carboxylate anion thereof), and salts thereof, found in the bile of an animal (e.g., a human), including, by way of non-limiting example, cholic acid, cholate, deoxycholic acid, deoxycholate, hyodeoxycholic acid, hyodeoxycholate, glycocholic acid, glycocholate, taurocholic acid, taurocholate, chenodeoxycholic acid, ursodeoxycholic acid, tauroursodeoxycholic acid, glycoursodeoxycholic acid, 7-B-methyl cholic acid, methyl lithocholic acid, chenodeoxycholate, lithocholic acid, lithocolate, and the like. Taurocholic acid and/or taurocholate are referred to herein as TCA. Any reference to a bile acid used herein includes reference to a bile acid, one and only one bile acid, one or more bile acids, or to at least one bile acid. Therefore, the terms “bile acid,” “bile salt,” “bile acid/salt,” “bile acids,” “bile salts,” and “bile acids/salts” are, unless otherwise indicated, utilized interchangeably herein. Any reference to a bile acid used herein includes reference to a bile acid or a salt thereof. Furthermore, pharmaceutically acceptable bile acid esters are optionally utilized as the “bile acids” described herein, e.g., bile acids conjugated to an amino acid (e.g., glycine or taurine). Other bile acid esters include, e.g., substituted or unsubstituted alkyl ester, substituted or unsubstituted heteroalkyl esters, substituted or unsubstituted aryl esters, substituted or unsubstituted heteroaryl esters, or the like. For example, the term “bile acid” includes cholic acid conjugated with either glycine or taurine: glycocholate and taurocholate, respectively (and salts thereof). Any reference to a bile acid used herein includes reference to an identical compound naturally or synthetically prepared. Furthermore, it is to be understood that any singular reference to a component (bile acid or otherwise) used herein includes reference to one and only one, one or more, or at least one of such components. Similarly, any plural reference to a component used herein includes reference to one and only one, one or more, or at least one of such components, unless otherwise noted. Moreover, as used herein, bile acid/salt mimics or mimetics described herein are compounds that mimic the agonist signaling properties of the bile acid/salt, especially at TGR5 (GPBAR1, BG37, Axor109) receptors. Examples includes those described in WO 2010/014836, which is incorporated herein for such disclosure. In some embodiments, bile acid mimetics include triterpenoid, such as oleanoic acid, ursolic acid, or the like.

The term “subject”, “patient” or “individual” are used interchangeably herein and refer to mammals and non-mammals, e.g., suffering from a disorder described herein. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The term “colon,” as used herein, includes the cecum, ascending colon, hepatic flexure, splenic flexure, descending colon, and sigmoid.

The term “composition,” as used herein includes the disclosure of both a composition and a composition administered in a method as described herein. Furthermore, in some embodiments, the composition of the present invention is or comprises a “formulation,” an oral dosage form or a rectal dosage form as described herein.

The terms “treat,” “treating” or “treatment,” and other grammatical equivalents as used herein, include alleviating, inhibiting or reducing symptoms, reducing or inhibiting severity of, reducing incidence of, reducing or inhibiting recurrence of, delaying onset of, delaying recurrence of, abating or ameliorating a disease or condition symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder (e.g., pancreatitis) being treated, and/or the eradication or amelioration of one or more of the physiological symptoms (e.g., abdominal pain) associated with the underlying disorder such that an improvement is observed in the patient.

The terms “prevent,” “preventing” or “prevention,” and other grammatical equivalents as used herein, include preventing additional symptoms, preventing the underlying causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

The term “non-systemic” or “minimally absorbed” as used herein refers to low systemic bioavailability and/or absorption of an administered compound. In some instances a non-systemic compound is a compound that is substantially not absorbed systemically. In some embodiments, ASBTI compositions described herein deliver the ASBTI to the distal ileum, colon, and/or rectum and not systemically (e.g., a substantial portion of the ASBTI is not systemically absorbed. In some embodiments, the systemic absorption of a non-systemic compound is <0.1%, <0.3%, <0.5%, <0.6%, <0.7%, <0.8%, <0.9%, <1%, <1.5%, <2%, <3%, or <5% of the administered dose (wt. % or mol %). In some embodiments, the systemic absorption of a non-systemic compound is <15% of the administered dose. In some embodiments, the systemic absorption of a non-systemic compound is <25% of the administered dose. In an alternative approach, a non-systemic ASBTI is a compound that has lower systemic bioavailability relative to the systemic bioavailability of a systemic ASBTI (e.g., compound 100A, 100C). In some embodiments, the bioavailability of a non-systemic ASBTI described herein is <30%, <40%, <50%, <60%, or <70% of the bioavailability of a systemic ASBTI (e.g., compound 100A, 100C).

In another alternative approach, the compositions described herein are formulated to deliver <10% of the administered dose of the ASBTI systemically. In some embodiments, the compositions described herein are formulated to deliver <20% of the administered dose of the ASBTI systemically. In some embodiments, the compositions described herein are formulated to deliver <30% of the administered dose of the ASBTI systemically. In some embodiments, the compositions described herein are formulated to deliver <40% of the administered dose of the ASBTI systemically. In some embodiments, the compositions described herein are formulated to deliver <50% of the administered dose of the ASBTI systemically. In some embodiments, the compositions described herein are formulated to deliver <60% of the administered dose of the ASBTI systemically. In some embodiments, the compositions described herein are formulated to deliver <70% of the administered dose of the ASBTI systemically. In some embodiments, systemic absorption is determined in any suitable manner, including the total circulating amount, the amount cleared after administration, or the like.

The term “ASBT inhibitor” refers to a compound that inhibits apical sodium-dependent bile transport or any recuperative bile salt transport. The term Apical Sodium-dependent Bile Transporter (ASBT) is used interchangeably with the term Ileal Bile Acid Transporter (IBAT).

The term “enhancing enteroendocrine peptide secretion” refers to a sufficient increase in the level of the enteroendocrine peptide agent to, for example, treat any disease or disorder described herein. In some embodiments, enhanced enteroendocrine peptide secretion reverses or alleviates symptoms of pancreatitis.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an O group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=11 and x=2, y=0. When x=2, the alkyl groups, taken together with the nitrogen to which they are attached, optionally form a cyclic ring system.

An “amide” is a chemical moiety with formula —C(O)NHR or —NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

As used herein, the term “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

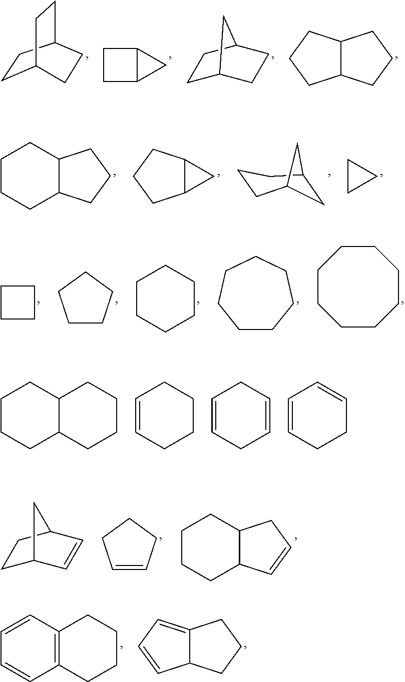

and the like. Monocyclic cycloalkyls include, but are not limited to, cyclopropyl, cyclobutyl, cyclopentyl, cyclohexyl, cycloheptyl, and cyclooctyl.

The term “heterocyclo” refers to heteroaromatic and heteroalicyclic groups containing one to four ring heteroatoms each selected from O, S and N. In certain instances, each heterocyclic group has from 4 to atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Illustrative examples of heteroaryl groups include the following moieties:

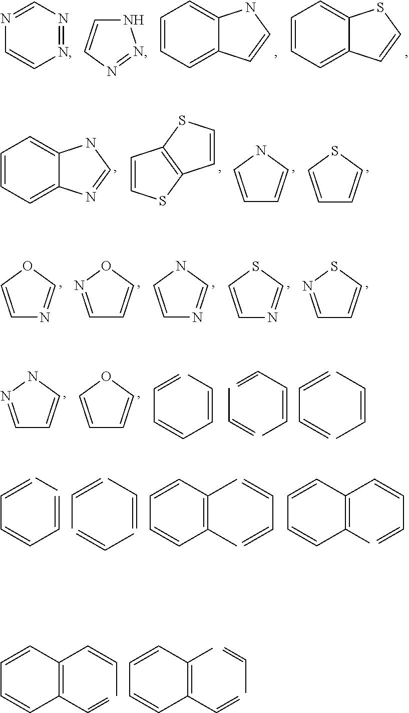

and the like.

A “heteroalicyclic” group or “heterocyclo” group refers to a cycloalkyl group, wherein at least one skeletal ring atom is a heteroatom selected from nitrogen, oxygen and sulfur. In various embodiments, the radicals are with an aryl or heteroaryl. Illustrative examples of heterocyclo groups, also referred to as non-aromatic heterocycles, include:

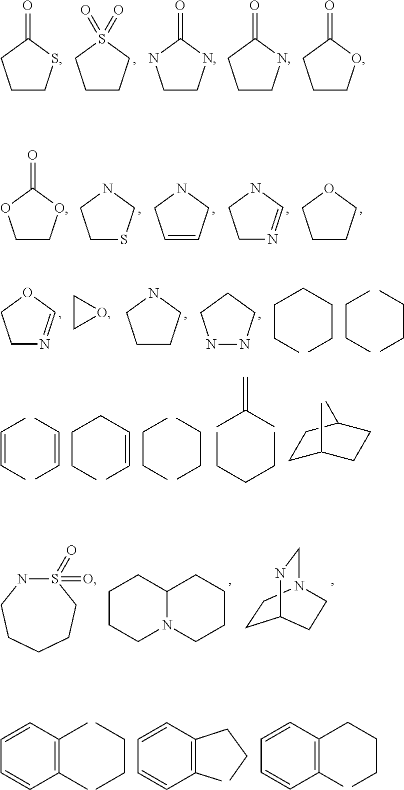

and the like. The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a —CN group.

An “isocyanato” group refers to a —NCO group.

A “thiocyanato” group refers to a —CNS group.

An “isothiocyanato” group refers to a —NCS group.

“Alkoyloxy” refers to a RC(═O)O— group.

“Alkoyl” refers to a RC(═O)— group.

The term “modulate,” as used herein refers to having some affect on (e.g., increasing, enhancing or maintaining a certain level).

The term “optionally substituted” or “substituted” means that the referenced group may be substituted with one or more additional group(s) individually and independently selected from C₁-C₆alkyl, C₃-C₈cycloalkyl, aryl, heteroaryl, C₂-C₆ heteroalicyclic, hydroxy, C₁-C₆ alkoxy, aryloxy, C₁-C₆alkylthio, arylthio, C₁-C₆alkylsulfoxide, arylsulfoxide, C₁-C₆alkylsulfone, arylsulfone, cyano, halo, C₂-C₈acyl, C₂-C₈acyloxy, nitro, C₁-C₆ haloalkyl, C₁-C₆ fluoroalkyl, and amino, including C₁-C₆alkylamino, and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(═O)—, —C(═O)NH—, S(═O)₂ NH—, —NHS(═O)₂—, —OC(═O)NH—, —NHC(═O)O—, —(C₁-C₆alkyl)-, or —(C₂-C₆ alkenyl)-; and each R^{s} is independently selected from H, (C₁-C₄alkyl), (C₃-C₈cycloalkyl), heteroaryl, aryl, and C₁-C₆ heteroalkyl. Optionally substituted non-aromatic groups may be substituted with one or more oxo (═O). The protecting groups that may form the protective derivatives of the above substituents are known to those of skill in the art and may be found in references such as Greene and Wuts, above. In some embodiments, alkyl groups described herein are optionally substituted with an O that is connected to two adjacent carbon atoms (i.e., forming an epoxide).

The term “therapeutically effective amount” or an “effective amount” as used herein, refers to a sufficient amount of a therapeutically active agent to provide a desired effect in a subject or individual. In some embodiments, a “therapeutically effective amount” or an “effective amount” of an enteroendocrine peptide secretion enhancing agent or an ABTI or an FXR agonist refers to a sufficient amount of the enteroendocrine peptide secretion enhancing agent or an ABTI or an FXR agonist to treat pancreatitis in a subject or individual. In some embodiments, a “therapeutically effective amount” or an “effective amount” of an enteroendocrine peptide secretion enhancing agent refers to a sufficient amount of an enteroendocrine peptide secretion enhancing agent or an ABTI or an FXR agonist to increase the secretion of enteroendocrine peptide(s) and/or bile acids in a subject or individual such that alleviation of symptoms of pancreatitis is observed.

### Enteroendocrine Cells (EEC)

Inventors have discovered that EEC plays a role in innate immunity and repair. Host defense against invading microbial organisms is maintained by an intact epithelial barrier and by the immune system. Immunity has innate and acquired components, recognizing microorganisms as non-self and triggering an immune response. Cells of the innate immune system principally sense microbial presence via activation of Toll-like receptors (TLR). TLR are differentially distributed in multiple cell types, but are chiefly expressed by dendritic cells, macrophages, and myofibroblasts TLRs recognize a broad range of pathogen derived components, signaling to induce the expression of pro-inflammatory genes and cytokines as a coordinated immune response. This, in conjunction with phagocytosis-mediated antigen presentation, instructs the development of antigen-specific adaptive immunity, especially via Th₁ cells. TLRs are also found on EEC. This assigns a novel role to EEC as innate immunity sensors, in addition to their canonical role as nutrient sensors.

### L-Cells

The epithelial barrier is also a key component in host defence. A further pre-proglucagon splice product, GLP-2, is secreted by enteroendocrine L-cells in the distal small intestine and has been shown to improve intestinal wound healing in a TGF-B (anti-inflammatory cytokine TGF-B), mediated process, small bowel responding better than large bowel. GLP-2 has also been shown to ameliorate the barrier dysfunction induced by experimental stress and food allergy. Again, L-cells are activated by luminal nutrients, and the barrier compromise observed in TPN may partly reflect its hyposecretion in the absence of enteral stimuli. Moreover, GLP-2 is also responsible, at least in part for growth and adaptation observed in short-bowel models. Therefore, abnormal enteroendocrine cells (EEC) function may predispose to GI inflammatory disorders, and the underlying nutrient-EEC-vagal pathways are targets in the injured gut as contemplated in the present embodiments.

L-cells are scattered throughout the epithelial layer of the gut from the duodenum to the rectum, with the highest numbers occurring in the ileum, colon, and rectum. They are characterized by an open-cell morphology, with apical microvilli facing into the gut lumen and secretory vesicles located adjacent to the basolateral membrane, and are therefore in direct contact with nutrients in the intestinal lumen. Furthermore, L-cells are located in close proximity to both neurons and the microvasculature of the intestine, thereby allowing the L-cell to be affected by both neural and hormonal signals. As well as Glucagon-Like Peptide 1 (GLP-1) and Glucagon-Like Peptide 2 (GLP-2), L-cells also secrete peptide YY (PYY), and glutamate. The cells are just one member of a much larger family of enteroendocrine cells that secrete a range of hormones, including ghrelin, GIP, cholecystokinin, somatostatin, and secretin, which are involved in the local coordination of gut physiology, as well as in playing wider roles in the control of cytokine release and/or controlling the adaptive process, attenuating intestinal injury, reducing bacterial translocation, inhibiting the release of free radical oxygen, or any combination thereof. L-cells are unevenly distributed in the gastrointestinal tract, within higher concentrations in the distal portion of the gastrointestinal tract (e.g., in the distal ileum, colon and rectum).

### Proglucagon Products

The proglucagon gene product is expressed in the L-cells of the small intestine, in beta-cells of the pancreas and in the central nervous system. Tissue-specific expression of isoforms of the enzyme prohormone convertase directs posttranslational synthesis of specific proglucagon-derived peptides in the L-cell and α-cell. Specifically, cleavage of proglucagon by prohormone convertase 1/3, which is expressed in the L-cell, forms GLP-1 and GLP-2, as well as the glucagon-containing peptides, glicentin and oxyntomodulin. In contrast, α-cell expression of prohormone convertase 2 forms glucagon, glicentin-related pancreatic peptide, and the major proglucagon fragment, which contains within its sequence both the GLP-1 and GLP-2 sequences.

### Pancreatic Polypeptide (PP)-Fold Peptides

The Pancreatic Polypeptide (PP)-fold peptides include Peptide YY (PYY), Pancreatic Polypeptide (PP) and Neuropeptide Y (NPY), which all share sequence homology and contain several tyrosine residues. They have a common tertiary structure which consists of an alpha-helix and polyproline helix, connected by a β-turn, resulting in a characteristic U-shaped peptide, the PP-fold.

Neuropeptide Y (NPY) is one of the most abundant neurotransmitters in the brain. Hypothalamic levels of NPY reflect the body's nutritional status, wherein the levels of hypothalamic NPY mRNA and NPY release increase with fasting and decrease after feeding.

Pancreatic Polypeptide (PP) is produced by cells at the periphery of the islets of the endocrine pancreas, and to a lesser extent in the exocrine pancreas, colon and rectum.

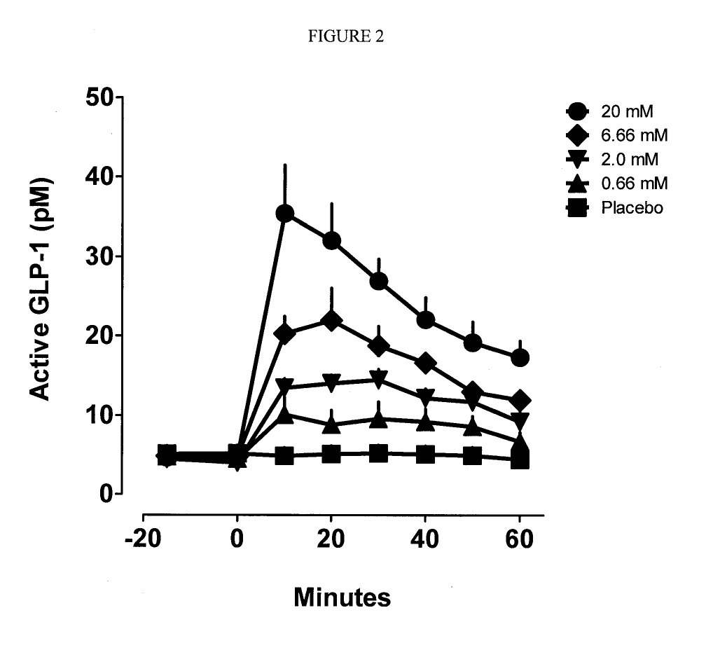

Peptide YY (PYY) is secreted predominantly from the distal gastrointestinal tract, particularly the ileum, colon and rectum. FIG. 2 illustrates the concentration of PYY at various locations in the gastrointestinal tract. Other signals, such as gastric acid, CCK and luminal bile salts, insulin-like growth factor 1, bombesin and calcitonin-gene-related peptide increase PYY levels, whereas gastric distension has no effect, and levels are reduced by GLP-1. The N-terminal of circulating PYY allows it to cross the blood-brain barrier.

In some embodiments, provided herein is a method of increasing circulating PYY levels by non-systemically administering an effective amount of an enteroendocrine peptide secretion enhancing agent (e.g., a bile acid) to an individual suffering from pancreatitis. In some embodiments, provided herein is a method of increasing circulating PYY levels by administering to the distal gastrointestinal tract (e.g., distal ileum, colon and/or rectum) an effective amount of an enteroendocrine peptide secretion enhancing agent (e.g., a bile acid).

### GLP-1

Glucagon-like peptide 1 (GLP-1) is an intestinal hormone that effects in the regulation of glycemia, stimulating glucose-dependent insulin secretion, proinsulin gene expression, and B-cell proliferative and anti-apoptotic pathways, as well as inhibiting glucagon release, gastric emptying, and food intake. The anorexigenic effect of GLP-1 is mediated by GLP-1 receptors which are present in both the NTS and hypothalamus, and in the pancreas, lung, brain, kidney, gastrointestinal tract and heart. Reduced secretion of GLP-1 contributes to the pathogenesis of pancreatitis.

The primary physiological stimulus of GLP-1 secretion from L-cells is ingestion of carbohydrates, luminal glucose (not systemic glucose) fat, and protein. Protein hydrolysate are also potent triggers of GLP-1 release, and certain amino acids such as, but not limited to, alanine, serine, glutamine, asparagine, and glycine stimulate GLP-1 release. Within the fat group, the long-chain unsaturated fatty acid and short-chain fatty acid subgroups are potent triggers of GLP-1 release, while the short-chain fatty acids also stimulate peptide YY release. In addition to luminal nutrients, intestinal peptides, neurotransmitters, as well as systemic hormones, modulate GLP-1 secretion. Such intestinal peptides include, but are not limited to, somatostatin (forms SS14 and SS28), and such neurotransmitters include, but are not limited to, acetylcholine and γ-aminobutyric acid (GABA) (both of which enhance GLP-1 release), and α- and β-adrenergic agonists, (which respectively inhibit and/or stimulate GLP-1 secretion from L-cells). Peripheral hormones that participate in energy homeostasis, such as the adipocyte hormone leptin, also stimulate GLP-1 release. Other GLP-1 secretegoues include bile acids/salts, insulin, gastrin-releasing peptide (GRP), several gut peptides including, but not limited to, Gastric Inhibitory Polypeptide (GIP) and calcitonin gene-related protein (CGRP). CGRP is a peptide found throughout the enteric nervous system. Thus, GLP-1 secretagogues include, but are not limited to, nutrients, neurotransmitters, neuropeptides, intestinal peptide, peripheral hormones, and bile acids/salts.

Within about 15 minutes of food ingestion the circulating GLP-1 levels increase and remain elevated for up to 3 hours, depending on the composition of the meal. Circulating GLP-1 exists in two equipotent forms, GLP-1^{7-36NH2} and GLP-1⁷⁻³⁷, with GLP-1^{7-36NH2} being the predominant form. Secreted GLP-1 is rapidly degraded by the ubiquitous enzyme dipeptidyl peptidase-4 (DPP-4), resulting in an extremely short half-life for GLP-1 of about 30 seconds to about 2 minutes. Therefore, levels of circulating GLP-1 are maintained by inhibiting DPP-4 activity, or alternatively, by enhancing GLP-1 secretion.

In some embodiments, provided herein is a method of increasing circulating GLP (e.g., GLP-1) levels by administering to the distal gastrointestinal tract (e.g., distal ileum, colon and/or rectum) an effective amount of an enteroendocrine peptide secretion enhancing agent (e.g., a bile acid) to an individual in need thereof.

In some embodiments, provided herein is a method of increasing circulating GLP-1 levels by non-systemically administering an effective amount of an ASBTI to an individual suffering from pancreatitis. In further embodiments, provided herein is a method of increasing circulating GLP-1 levels by administering a combination of an ASBTI and a DPP-4 inhibitor to an individual in need thereof. Increased levels of GLP-1 modify (e.g., reduce) secretion of pancreatic enzymes thereby alleviating symptoms of pancreatitis (e.g., abdominal pain, inflammation of the pancreas).

### GLP-2

Glucagon-like peptide-2 (GLP-2) is a 33 amino acid peptide, co-secreted along with GLP-1 from intestinal endocrine cells in the small and large intestine. GLP-2 exhibits a short t1/2 in vivo, due to rapid inactivation by DPP-4. Thus DPP-4 inhibitors will potentiate the action of exogenous and endogenous GLP-2, along with GLP-1.

### Enteroendocrine Peptide Secretion Enhanced Treatment

The methods and composition described herein use, by way of non-limiting example, the administration of bile acids/salts and bile acids/salts mimics to modulate (e.g., increase) the circulating levels of GLP-1. In certain embodiments of the present invention, such administration induces inflammation in pancreas.

### Bile Acid

Bile contains water, electrolytes and a numerous organic molecules including bile acids, cholesterol, phospholipids and bilirubin. Bile is secreted from the liver and stored in the gall bladder, and upon gall bladder contraction, due to ingestion of a fatty meal, bile passes through the bile duct into the intestine. Bile acids are critical for digestion and absorption of fats and fat-soluble vitamins in the small intestine. Adult humans produce 400 to 800 mL of bile daily. The secretion of bile can be considered to occur in two stages. Initially, hepatocytes secrete bile into canaliculi, from which it flows into bile ducts and this hepatic bile contains large quantities of bile acids, cholesterol and other organic molecules. Then, as bile flows through the bile ducts, it is modified by addition of a watery, bicarbonate-rich secretion from ductal epithelial cells. Bile is concentrated, typically five-fold, during storage in the gall bladder.

The flow of bile is lowest during fasting, and a majority of that is diverted into the gallbladder for concentration. When chyme from an ingested meal enters the small intestine, acid and partially digested fats and proteins stimulate secretion of cholecystokinin and secretin, both of which are important for secretion and flow of bile. Cholecystokinin (cholecysto=gallbladder and kinin=movement) is a hormone which stimulates contractions of the gallbladder and common bile duct, resulting in delivery of bile into the gut. The most potent stimulus for release of cholecystokinin is the presence of fat in the duodenum. Secretin is a hormone secreted in response to acid in the duodenum, and it simulates biliary duct cells to secrete bicarbonate and water, which expands the volume of bile and increases its flow out into the intestine.

Bile acids are derivatives of cholesterol. Cholesterol, ingested as part of the diet or derived from hepatic synthesis, are converted into bile acids in the hepatocyte. Examples of such bile acids include cholic and chenodeoxycholic acids, which are then conjugated to an amino acid (such as glycine or taurine) to yield the conjugated form that is actively secreted into cannaliculi. The most abundant of the bile salts in humans are cholate and deoxycholate, and they are normally conjugated with either glycine or taurine to give glycocholate or taurocholate respectively.

Free cholesterol is virtually insoluble in aqueous solutions, however in bile it is made soluble by the presence of bile acids and lipids. Hepatic synthesis of bile acids accounts for the majority of cholesterol breakdown in the body. In humans, roughly 500 mg of cholesterol are converted to bile acids and eliminated in bile every day. Therefore, secretion into bile is a major route for elimination of cholesterol. Large amounts of bile acids are secreted into the intestine every day, but only relatively small quantities are lost from the body. This is because approximately 95% of the bile acids delivered to the duodenum are absorbed back into blood within the ileum, by a process is known as “Enterohepatic Recirculation”.

Venous blood from the ileum goes straight into the portal vein, and hence through the sinusoids of the liver. Hepatocytes extract bile acids very efficiently from sinusoidal blood, and little escapes the healthy liver into systemic circulation. Bile acids are then transported across the hepatocytes to be resecreted into canaliculi. The net effect of this enterohepatic recirculation is that each bile salt molecule is reused about 20 times, often two or three times during a single digestive phase. Bile biosynthesis represents the major metabolic fate of cholesterol, accounting for more than half of the approximate 800 mg/day of cholesterol that an average adult uses up in metabolic processes. In comparison, steroid hormone biosynthesis consumes only about 50 mg of cholesterol per day. Much more that 400 mg of bile salts is required and secreted into the intestine per day, and this is achieved by re-cycling the bile salts. Most of the bile salts secreted into the upper region of the small intestine are absorbed along with the dietary lipids that they emulsified at the lower end of the small intestine. They are separated from the dietary lipid and returned to the liver for re-use. Re-cycling thus enables 20-30 g of bile salts to be secreted into the small intestine each day.

Bile acids are amphipathic, with the cholesterol-derived portion containing both hydrophobic (lipid soluble) and polar (hydrophilic) moieties while the amino acid conjugate is generally polar and hydrophilic. This amphipathic nature enables bile acids to carry out two important functions: emulsification of lipid aggregates and solubilization and transport of lipids in an aqueous environment. Bile acids have detergent action on particles of dietary fat which causes fat globules to break down or to be emulsified. Emulsification is important since it greatly increases the surface area of fat available for digestion by lipases which cannot access the inside of lipid droplets. Furthermore, bile acids are lipid carriers and are able to solubilize many lipids by forming micelles and are critical for transport and absorption of the fat-soluble vitamins.

### Pharmaceutical Compositions and Methods of Use

In some embodiments, compositions described herein are administered for delivery of enteroendocrine peptide secretion enhancing agents to a subject or individual. In certain embodiments, any compositions described herein are formulated for ileal, rectal and/or colonic delivery. In more specific embodiments, the composition is formulated for non-systemic or local delivery to the rectum and/or colon. It is to be understood that as used herein, delivery to the colon includes delivery to sigmoid colon, transverse colon, and/or ascending colon. In still more specific embodiments, the composition is formulated for non-systemic or local delivery to the rectum and/or colon is administered rectally. In other specific embodiments, the composition is formulated for non-systemic or local delivery to the rectum and/or colon is administered orally.

In some embodiments, provided herein is a composition comprising an enteroendocrine peptide secretion enhancing agent and, optionally, a pharmaceutically acceptable carrier for alleviating symptoms of pancreatitis in an individual.

In certain embodiments, the composition comprises an enteroendocrine peptide secretion enhancing agent and an absorption inhibitor. In specific embodiments, the absorption inhibitor is an inhibitor that inhibits the absorption of the (or at least one of the) specific enteroendocrine peptide secretion enhancing agent with which it is combined. In some embodiments, the composition comprises an enteroendocrine peptide secretion enhancing agent, an absorption inhibitor and a carrier (e.g., an orally suitable carrier or a rectally suitable carrier, depending on the mode of intended administration). In certain embodiments, the composition comprises an enteroendocrine peptide secretion enhancing agent, an absorption inhibitor, a carrier, and one or more of a cholesterol absorption inhibitor, an enteroendocrine peptide, a peptidase inhibitor, a spreading agent, and a wetting agent.

In certain embodiments enteroendocrine peptide secretion enhancing agents are selected from, by way of non-limiting example, bile acids, bile acid mimic and/or modified bile acids. In more specific embodiments, compositions described herein are formulated for non-systemic or local delivery of a bile acid, bile acid mimic and/or modified bile acid (as the active component or components) to the rectum and/or colon, including the sigmoid colon, transverse colon, and/or ascending colon. In certain embodiments, the compositions described herein are administered rectally for non-systemic or local delivery of the bile acid active component to the rectum and/or colon, including the sigmoid colon, transverse colon, and/or ascending colon. In other embodiments, the compositions described herein are administered orally for non-systemic delivery of the bile salt active component to the rectum and/or colon, including the sigmoid colon, transverse colon, and/or ascending colon. In specific embodiments, compositions formulated for oral administration are, by way of non-limiting example, enterically coated or formulated oral dosage forms, such as, tablets and/or capsules. It is to be understood that the terms “subject” and “individual” are utilized interchangeably herein and include, e.g., humans and human patients in need of treatment.

### Enteroendocrine Peptide Enhancing Agents

In some embodiments, enteroendocrine peptide enhancing agents provided herein include, by way of non-limiting example, enteroendocrine peptide secretion (e.g., of the L-cells) enhancing agents, inhibitors of degradation of enteroendocrine peptides (e.g., of the L-cells), or combinations thereof.

In certain embodiments, the enteroendocrine peptide secretion enhancing agents used in the methods and compositions described herein include, by way of non-limiting example, a steroid acid or a nutrient. In specific embodiments, the steroid acid or nutrient described herein is a steroid acid or nutrient that enhances the secretion of an enteroendocrine peptide. In specific embodiments, the steroid acid is an oxidize cholesterol acid. In some embodiments, an enteroendocrine peptide secretion enhancing agent, bile acid, or bile acid mimic used in any composition or method described herein is a compound of Formula VII:

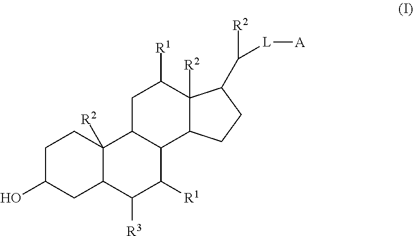

In certain embodiments, each R¹ is independently H, OH, O-lower alkyl (e.g., OCH₃, or OEt). In some embodiments, each R¹ is independently H, OH, lower (e.g., C₁-C₆ or C₁-C₃) alkyl, or lower (e.g., C₁-C₆ or C₁-C₃) heteroalkyl. In certain embodiments, L is a substituted or unsubstituted alkyl or substituted or unsubstituted heteroalkyl. In some embodiments, R² is H, OH, lower alkyl, or lower heteroalkyl (e.g., OMe). In certain embodiments, R³ is H, OH, O-lower alkyl, lower alkyl, or lower heteroalkyl (e.g., OMe). In some embodiments, A is COOR⁴, S(O)ₙR⁴, or OR⁵. In certain embodiments, R⁴ is H, an anion, a pharmaceutically acceptable cation (e.g., an alkali metal cation, alkaline earth metal cation, or any other pharmaceutically acceptable cation) substituted or unsubstituted alkyl, substituted or unsubstituted heteroalkyl, substituted or unsubstituted aryl, substituted or unsubstituted heteroaryl, an amino acid, or the like; and n is 1-3. Each R⁵ is independently selected from lower alkyl and H.

In specific embodiments, L is unsubstituted branched or straight chain alkyl. In more specific embodiments, L is unsubstituted branched or straight chain lower alkyl. In some embodiments, L is (CR⁵₂)ₘ—CONR⁵—(CR⁵₂)ₚ. Each m is 1-6 and n is 1-6. In specific embodiments, m is 2 and n is 1. In other specific embodiments, m is 2 and n is 2. In certain embodiments, A is COOH or COO—. In some embodiments, A is SO₃H or SO₃—.

In specific embodiments, the compound of Formula VII has a structure represented by:

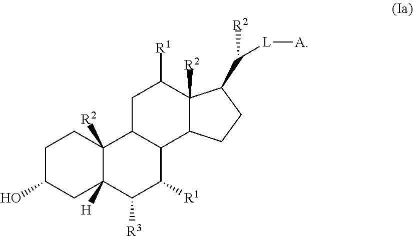

In some embodiments, bile acid mimics include, by way of non-limiting example, 6-methyl-2-oxo-4-thiophen-2-yl-1,2,3,4-tetrahydro-phyrimidine-5-carboxylic acid benzyl ester (or TGR5-binding analogs thereof), oleanolic acid (or TGR5-binding analogs thereof), crataegolic acid, 6α-ethyl-23(S)-methylcholic acid (S-EMCA, INT-777), (3R)-3-Hydroxy-3-(2-propen-1-yl)-lup-20(29)-en-28-oic acid hydrate (RG-239), or the like.

In some embodiments, a bile acid mimic is

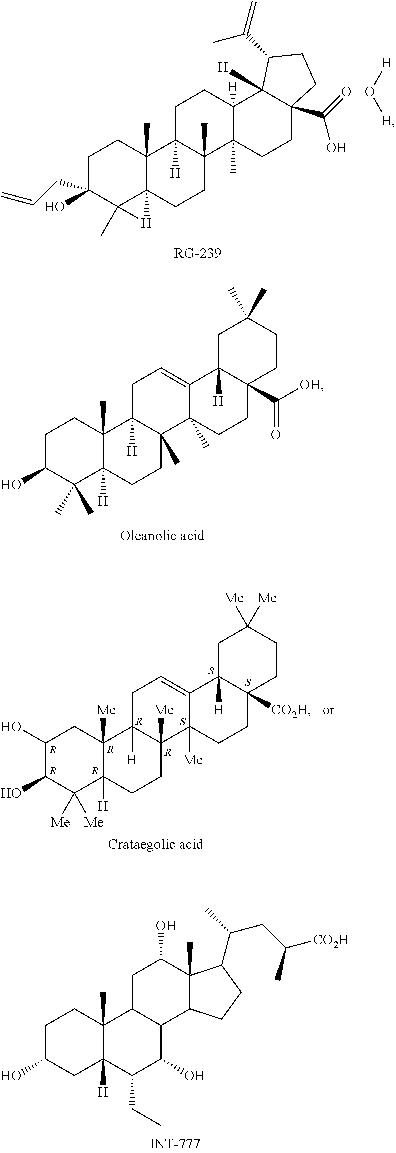

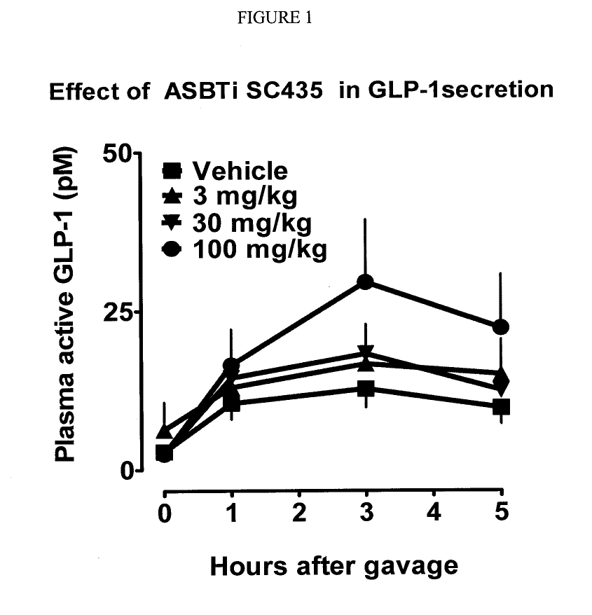

In certain embodiments, enteroendocrine peptide secretion enhancing agents used in the methods and compositions described herein enhance the secretion of an enteroendocrine peptide secreted by L-cells (e.g., GLP-1, GLP-2, PYY, and the like). FIG. 1 (FIGS. 1A and 1B) illustrates the response of enteroendocrine peptides to administration of bile salts.

In some embodiments, the enteroendocrine peptide secretion enhancing agent is a steroid acid, such as a bile acid/salt, a bile acid/salt mimic, a modified bile acid/salt, or a combination thereof. The bile acids or salts thereof used in the methods and compositions described herein include, by way of non-limiting example, cholic acid, deoxycholic acid, glycocholic acid, glycodeoxycholic acid, taurocholic acid, taurodihydrofusidate, taurodeoxycholic acid, cholate, glycocholate, deoxycholate, taurocholate, taurodeoxycholate, chenodeoxycholic acid, ursodeoxycholic acid, tauroursodeoxycholic acid, glycoursodeoxycholic acid, 7-B-methyl cholic acid, methyl lithocholic acid, and combinations thereof. In certain embodiments, bile salts used in the methods and compositions described herein are pharmaceutically acceptable salts including, by way of non-limiting example, the sodium and potassium salts thereof. In specific embodiments, the enteroendocrine peptide secretion enhancing agent is a pharmaceutically acceptable bile acid salt including, by way of non-limiting example, sodium glycocholate, sodium taurocholate and combinations thereof. In some embodiments, more than one bile acid and/or salt is used in a methods and/or composition described herein. In certain embodiments, the bile acid/salt used herein has a low or relatively low solubility in water.

Although bile acids facilitate digestion and absorption of lipids in the small intestine, they are generally used in pharmaceutical formulations as excipients. As excipients, bile acids find uses as surfactants and/or as agents that enhance the transfer of active components across mucosal membranes, for systemic delivery of a pharmaceutically active compound. In certain embodiments of the methods and pharmaceutical compositions described herein, however, a bile acid, a bile acid mimic and/or a modified bile acid is the active agent used to enhance secretion of enteroendocrine peptides.

In certain specific embodiments, the enteroendocrine peptide secretion enhancing agents used in the methods and compositions described herein are modified bile acids/salts. In certain embodiments, the bile acid/salt is modified in such a way so as to inhibit absorption of the bile acid/salt across the rectal or colonic mucosa.

In certain embodiments, the enteroendocrine peptide secretion enhancing agents described herein are a glucagon-like peptide secretion enhancing agent. In a specific embodiment, the glucugen-like peptide secretion enhancing agent is a bile acid, a bile acid mimic or a modified bile acid. In some embodiments, the glucagon-like peptide secretion enhancing agents are selected from, by way of non-limiting example, glucagon-like peptide-1 (GLP-1) secretion enhancing agents or glucagon-like peptide-2 (GLP-2) secretion enhancing agents. In some embodiments, the glucagon-like peptide secretion enhancing agents enhance both GLP-1 and GLP-2. In a specific embodiment, the GLP-1 and/or GLP-2 secretion enhancing agent is selected from bile acids, bile acid mimics or modified bile acids.

In certain embodiments, the enteroendocrine peptide secretion enhancing agent described herein is a pancreatic polypeptide-fold peptide secretion enhancing agent. In more specific embodiments, the pancreatic polypeptide-fold peptide secretion enhancing agent is selected from, by way of non-limiting example, peptide YY (PYY) secretion enhancing agents. In specific embodiments, the pancreatic polypeptide-fold peptide secretion enhancing agent or the PYY secretion enhancing agent is selected from a bile acid, a bile acid mimic, a modified bile acid or a fatty acid or salt thereof (e.g., a short chain fatty acid).

In some embodiments, the enteroendocrine peptide secretion enhancing agent is selected from, by way of non-limiting example, carbohydrates, glucose, fats, and proteins. In certain embodiments, the enteroendocrine peptide secretion enhancing agent is selected from fatty acids, including long chain fatty acids and short chain fatty acids. Short chain fatty acids and salts include, by way of non-limiting example, propionic acid, butyric acid, propionate, and butyrate.

In some embodiments, the enteroendocrine peptide secretion enhancing agent is selected from, by way of non-limiting example, carbohydrates, glucose, fat, protein, protein hydrolysate, amino acids, nutrients, intestinal peptides, peripheral hormones that participate in energy homeostasis, such as the adipocyte hormone leptin, bile acids/salts, insulin, gastrin-releasing peptide (GRP), gut peptides, gastric acid, CCK, insulin-like growth factor 1, bombesin, calcitonin-gene-related peptide and combinations thereof that enhance the secretion of enteroendocrine peptides.

In certain embodiments, the inhibitors of degradation of L-cell enteroendocrine peptide products include DPP-IV inhibitors, TGR5 modulators (e.g., TGR5 agonists), or combinations thereof. In certain instances, the administration of a DPP-IV inhibitor in combination with any of the compounds disclosed herein reduces or inhibits degradation of GLP-1 or GLP-2. In certain instances, administration of a TGR5 agonist in combination with any of the compounds disclosed herein enhances the secretion of enteroendocrine peptide products from L-cells. In some instances, the enteroendocrine peptide enhancing agent agonizes or partially agonizes bile acid receptors (e.g., TGR5 receptors or Farnesoid-X receptors) on in the gastrointestinal tract.

DPP-IV inhibitors include (2S)-1-{2-[(3-hydroxy-1-adamantyl)amino]acetyl}pyrrolidine-2-carbonitrile (vildagliptin), (3R)-3-amino-1-[9-(trifluoromethyl)-1,4,7,8-tetrazabicyclo[4.3.0]nona-6,8-dien-4-yl]-4-(2,4,5-trifluorophenyl)butan-1-one (sitagliptin), (1S,3S,5S)-2-[(2S)-2-amino-2-(3-hydroxy-1-adamantyl)acetyl]-2-azabicyclo[3.1.0]hexane-3-carbonitrile (saxagliptin), and 2-({6-[(3R)-3-aminopiperidin-1-yl]-3-methyl-2,4-dioxo-3,4-dihydropyrimidin-1(2H)-yl}methyl)benzonitrile (alogliptin). TGR5 modulators (e.g., agonists) include the compounds disclosed in, e.g, WO2008/091540, WO 2008067219 and US Appl. No. 2008/0221161, the TGR5 modulators (e.g., agonists) of which are hereby incorporated herein by reference.

In some embodiments, the enteroendocrine peptide secretion enhancing agents used in the methods and compositions described herein may or may not be substrates for bile acid scavenger systems. In some embodiments, the enteroendocrine peptide secretion enhancing agents may not form micelles and/or assist in fat absorption. In certain embodiments, the enteroendocrine peptide secretion enhancing agents may or may not enhance permeability and/or promote inflammation. In certain embodiments, the enteroendocrine peptide secretion enhancing agent may not irritate the bowel or promote diarrhea. In some embodiments, the enteroendocrine peptide secretion enhancing agent is selected from, by way of non-limiting example, toll or toll-like receptor ligands.

### FXR Agonists

In some embodiments, FXR agonist is GW4064, GW9662, INT-747, T0901317, WAY-362450, fexaramine, a cholic acid, a deoxycholic acid, a glycocholic acid, a glycodeoxycholic acid, a taurocholic acid, a taurodihydrofusidate, a taurodeoxycholic acid, a cholate, a glycocholate, a deoxycholate, a taurocholate, a taurodeoxycholate, a chenodeoxycholic acid, an ursodeoxycholic acid, a tauroursodeoxycholic acid, a glycoursodeoxycholic acid, a 7-B-methyl cholic acid, a methyl lithocholic acid.

### Absorption Inhibitors

In certain embodiments, the compositions described herein are and the methods described herein include administering a composition that is formulated for the non-systemic delivery of enteroendocrine peptide secretion enhancing agents to the rectum and/or colon (sigmoid, transverse, and/or ascending colon). As previously discussed, enteroendocrine peptide secretion enhancing agents include, by way of non-limiting example, bile acids, bile salts, bile acid mimics, bile salt mimics, modified bile acids, modified bile salts and combinations thereof. In certain embodiments, the composition described herein as being formulated for the non-systemic delivery of enteroendocrine peptide secretion enhancing agents further includes an absorption inhibitor. As used herein, an absorption inhibitor includes an agent or group of agents that inhibit absorption of the enteroendocrine peptide secretion enhancing agent across the rectal or colonic mucosa. In specific embodiments, the absorption inhibitor is an absorption inhibitor that inhibits the absorption of the specific enteroendocrine peptide secretion enhancing agent with which it is combined.

Suitable bile acid absorption inhibitors (also described herein as absorption inhibiting agents) include, by way of non-limiting example, anionic exchange matrices, polyamines, quaternary amine containing polymers, quaternary ammonium salts, polyallylamine polymers and copolymers, colesevelam, colesevelam hydrochloride, CholestaGel (N,N,N-trimethyl-6-(2-propenylamino)-1-hexanaminium chloride polymer with (chloromethyl)oxirane, 2-propen-1-amine and N-2-propenyl-1-decanamine hydrochloride), cyclodextrins, chitosan, chitosan derivatives, carbohydrates which bind bile acids, lipids which bind bile acids, proteins and proteinaceous materials which bind bile acids, and antibodies and albumins which bind bile acids. Suitable cyclodextrins include those that bind bile acids such as, by way of non-limiting example, β-cyclodextrin and hydroxypropyl-β-cyclodextrin. Suitable proteins, include those that bind bile acids such as, by way of non-limiting example, bovine serum albumin, egg albumin, casein, α^{□}-acid glycoprotein, gelatin, soy proteins, peanut proteins, almond proteins, and wheat vegetable proteins.

In certain embodiments the absorption inhibitor is cholestyramine. In specific embodiments, cholestyramine is combined with a bile acid. Cholestyramine, an ion exchange resin, is a styrene polymer containing quaternary ammonium groups crosslinked by divinylbenzene. In other embodiments, the absorption inhibitor is colestipol. In specific embodiments, colestipol is combined with a bile acid. Colestipol, an ion exchange resin, is a copolymer of diethylenetriamine and 1-chloro-2,3-epoxypropane.

In certain embodiments of the compositions and methods described herein the enteroendocrine peptide secretion enhancing agent is linked to an absorption inhibitor, while in other embodiments the enteroendocrine peptide secretion enhancing agent and the absorption inhibitor are separate molecular entities. In specific embodiments the bile acid, bile acid mimic or the modified bile acid is linked to a bile acid adsorption inhibitor described herein.

### Cholesterol Absorption Inhibitors

In certain embodiments, a composition described herein optionally includes at least one cholesterol absorption inhibitor. Suitable cholesterol absorption inhibitors include, by way of non-limiting example, ezetimibe (SCH 58235), ezetimibe analogs, ACT inhibitors, stigmastanyl phosphorylcholine, stigmastanyl phosphorylcholine analogues, β-lactam cholesterol absorption inhibitors, sulfate polysaccharides, neomycin, plant sponins, plant sterols, phytostanol preparation FM-VP4, Sitostanol, P -sitosterol, acyl-CoA:cholesterol-O-acyltransferase (ACAT) inhibitors, Avasimibe, Implitapide, steroidal glycosides and the like. Suitable enzetimibe analogs include, by way of non-limiting example, SCH 48461, SCH 58053 and the like. Suitable ACT inhibitors include, by way of non-limiting example, trimethoxy fatty acid anilides such as C1-976, 3-[decyldimethylsilyl]-N-[2-(4-methylphenyl)-1-phenylethyl]-propanamide, melinamide and the like. P -lactam cholesterol absorption inhibitors include, by way of non-limiting example, (3R-4S)-1,4-bis-(4-methoxyphenyl)-3-(3-phenylpropyl)-2-azetidinone and the like.

### Enteroendocrine Peptides

In certain embodiments, the compositions described herein optionally include at least one enteroendocrine peptide. Suitable enteroendocrine peptides include, by way of non-limiting example, glucagon-like peptides GLP-1 and/or GLP-2, or pancreatic polypeptide -fold peptides pancreatic polypeptide (PP), neuropeptide Y (NPY) and/or peptide YY (PYY).

### Peptidase Inhibitors

In some embodiments, the compositions described herein optionally include at least one peptidase inhibitor. Such peptidase inhibitors include, but are not limited to, dipeptidyl peptidase-4 inhibitors (DPP-4), neutral endopeptidase inhibitors, and converting enzyme inhibitors. Suitable dipeptidyl peptidase-4 inhibitors (DPP-4) include, by way of non-limiting example, Vildaglipti, 2S)-1-{2-[(3-hydroxy-1-adamantyl)amino]acetyl}pyrrolidine-2-carbonitrile, Sitagliptin, (3R)-3-amino-1-[9-(trifluoromethyl)-1,4,7,8-tetrazabicyclo[4.3.0]nona-6,8-dien-4-yl]-4-(2,4,5-trifluorophenyl)butan-1-one, Saxagliptin, and (1S,3S,5S)-2-[(2S)-2-amino-2-(3-hydroxy-1-adamantyl)acetyl]-2-azabicyclo[3.1.0]hexane-3-carbonitrile. Such neutral endopeptidase inhibitors include, but are not limited to, Candoxatrilat and Ecadotril.

### Spreading Agents/Wetting Agents

In certain embodiments, the composition described herein optionally comprises a spreading agent. In some embodiments, a spreading agent is utilized to improve spreading of the composition in the colon and/or rectum. Suitable spreading agents include, by way of non-limiting example, hydroxyethylcellulose, hydroxypropymethyl cellulose, polyethylene glycol, colloidal silicon dioxide, propylene glycol, cyclodextrins, microcrystalline cellulose, polyvinylpyrrolidone, polyoxyethylated glycerides, polycarbophil, di-n-octyl ethers, Cetiol™OE, fatty alcohol polyalkylene glycol ethers, Aethoxal™B), 2-ethylhexyl palmitate, Cegesoft™C 24), and isopropyl fatty acid esters.

In some embodiments, the compositions described herein optionally comprise a wetting agent. In some embodiments, a wetting agent is utilized to improve wettability of the composition in the colon and rectum. Suitable wetting agents include, by way of non-limiting example, surfactants. In some embodiments, surfactants are selected from, by way of non-limiting example, polysorbate (e.g., 20 or 80), stearyl hetanoate, caprylic/capric fatty acid esters of saturated fatty alcohols of chain length C₁₂-C₁₈, isostearyl diglycerol isostearic acid, sodium dodecyl sulphate, isopropyl myristate, isopropyl palmitate, and isopropyl myristate/isopropyl stearate/isopropyl palmitate mixture.

### Methods

Provided herein, in certain embodiments, are methods for treating pancreatitis and/or symptoms of pancreatitis (e.g., abdominal pain) comprising administration of a therapeutically effective amount of an ASBTI and/or an enteroendocrine peptide enhancing agent and/or a FXR agonist to an individual in need thereof. Provided herein, in certain embodiments, are methods for treating pancreatitis and/or symptoms of pancreatitis (e.g., abdominal pain) comprising contacting the gastrointestinal tract, including the distal ileum and/or the colon and/or the rectum, of an individual in need thereof with an ASBTI and/or an enteroendocrine peptide enhancing agent and/or a FXR agonist. Also provided herein are methods for reducing intraenterocyte bile acids, reducing activity and/or secretion of pancreatic enzymes, of an individual comprising administration of a therapeutically effective amount of an ASBTI and/or an enteroendocrine peptide enhancing agent and/or a FXR agonist to an individual in need thereof.

In some embodiments, provided herein is a method of treating pancreatitis and/or symptoms of pancreatitis (e.g., abdominal pain) in an individual comprising delivering to ileal, colon, and/or rectal L-cells of the individual a therapeutically effective amount of any ASBTI and/or enteroendocrine peptide secretion enhancing agent described herein. In certain embodiments, the therapeutically effective amount of enteroendocrine peptide secretion enhancing agent stimulates or activates the L-cells to which the enteroendocrine peptide secretion enhancing agent is administered.

Provided herein are methods for stimulating L-cells in the distal gastrointestinal tract, including L-cells in the distal ileum and/or colon and/or rectum, of an individual comprising administration of a therapeutically effective amount of an ASBTI and/or an enteroendocrine peptide enhancing agent and/or a FXR agonist to an individual in need thereof. Also provided herein is a method of promoting stimulation of L-cell secretion in an individual in need thereof, the method comprising orally or rectally administering an effective amount of a minimally absorbed bile acid, bile salt, or mimetic thereof. In specific instances, the individual in need thereof is suffering from a disorder (e.g., pancreatitis) ameliorated by L-cell secreted products. Also provided herein is a method of promoting stimulation of L-cell secretion in an individual in need thereof, the method comprising orally administering an effective amount of a minimally absorbed ASBIT or salt thereof. In specific instances, the individual in need thereof is suffering from a disorder (e.g., pancreatitis) ameliorated by L-cell secreted products.

In certain embodiments, increased L-cell secretion of enteroendocrine peptides is associated with reduced secretion of pancreatic enzymes. In certain instances, increased L-cell secretion of enteroendocrine peptides is associated with protection of the pancreas (e.g., via decrease in production of inflammatory cytokines). In some embodiments, increased L-cell secretion of enteroendocrine peptides is associated with a reduction in severity of symptoms associated with pancreatitis (e.g., abdominal pain).

Provided herein are methods for increasing the concentration of bile acids and salts thereof in the vicinity of L-cells lining the gastrointestinal tract, including L-cells in the distal ileum, and/or the colon and/or the rectum of an individual, comprising administration of a therapeutically effective amount of an ASBTI and/or an enteroendocrine peptide enhancing agent and/or a FXR agonist to an individual in need thereof. In some of the aforementioned embodiments, the ASBTI and/or an enteroendocrine peptide enhancing agent and/or a FXR agonist is contacted with the distal ileum of the individual in need thereof. In some of the aforementioned embodiments, the ASBTI is not absorbed systemically. In some other embodiments, the ASBTI is absorbed systemically.

In some embodiments of the methods provided herein, inhibition of bile acid transporters and/or bile acid recycling increases the concentration of bile acids in the vicinity of L-cells to concentrations that are higher than physiological levels of bile acids in individuals that have not been treated with an ASBTI and/or an enteroendocrine peptide enhancing agent and/or a FXR agonist. In certain embodiments, an increase in concentration of bile acids in the intestinal lumen of an individual is more effective for healing of the pancreas that has been injured by auto-digestion compared to baseline concentrations of bile acids in the intestinal lumen of the individual. In certain embodiments, an increase in concentration of bile acids in the intestinal lumen of an individual is more effective for reducing symptoms of pancreatitis and/or symptoms thereof and/or duration of illness compared to baseline concentrations of bile acids in the intestinal lumen of the individual.

In some embodiments of the methods described herein, an increase in concentration of bile acids in the vicinity of L-cell increases the secretion of enteroendocrine peptides, including GLP-1, GLP-2, PYY and/or oxyntomodulin from L-cells. In some instances a higher concentration of GLP-1 and/or GLP-2 and/or PYY and/or oxynotmodulin in the blood and/or plasma of an individual, induces suppression of pancreatic secretions and/or reduces activation of pancreatic enzymes, reduces intraenterocyte bile acids, and/or reduces damage to pancreas caused by auto-digestion.

Provided herein are methods for reducing damage to the pancreas comprising administration of a therapeutically effective amount of an ASBTI and/or an enteroendocrine peptide enhancing agent and/or a FXR agonist to an individual in need thereof (e.g., an individual suffering from pancreatitis).

Provided herein are methods for reducing pain associated with pancreatitis comprising administration of a therapeutically effective amount of an ASBTI and/or an enteroendocrine peptide enhancing agent and/or a FXR agonist to an individual in need thereof (e.g., an individual suffering from pancreatitis).

Provided herein are methods for preventing, reducing occurrence of, or delaying onset of pancreatitis after a pancreato-biliary surgical procedure comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

Provided herein are methods for preventing, reducing occurrence of, or delaying onset of pancreatitis as a complication of surgery (e.g., Endoscopic Retrograde Cholangiopancreatography Procedure (ERCP) procedure) comprising administration of a therapeutically effective amount of an ASBTI and/or an enteroendocrine peptide enhancing agent and/or a FXR agonist to an individual in need thereof (e.g., an individual who has undergone an ERCP procedure).

In certain embodiments, provided herein are methods for reducing intraenterocyte bile acids comprising administration of a therapeutically effective amount of an ASBTI and/or an enteroendocrine peptide enhancing agent and/or a FXR agonist to an individual in need thereof.

In some embodiments, the methods provide for inhibition of bile salt recycling upon administration of any of the compounds described herein to an individual. In some embodiments, an ASBTI and/or an enteroendocrine peptide enhancing agent and/or a FXR agonist described herein is systemically absorbed upon administration. In some embodiments, an ASBTI and/or an enteroendocrine peptide enhancing agent and/or a FXR agonist described herein is not absorbed systemically. In some embodiments, an ASBTI and/or an enteroendocrine peptide enhancing agent and/or a FXR agonist described herein is administered to the individual orally, enterically or rectally. In some embodiments, an ASBTI and/or an enteroendocrine peptide enhancing agent and/or a FXR agonist described herein is delivered and/or released in the distal ileum of an individual. In some embodiments, an ASBTI and/or an enteroendocrine peptide enhancing agent and/or a FXR agonist described herein increases the concentration of bile acids in the distal ileum, the colon and/or the rectum thereby increasing secretion of enteroendocrine peptide products from L-cells in the gastrointestinal tract. In certain instances administration of a therapeutically effective amount of an ASBTI and/or an enteroendocrine peptide enhancing agent and/or a FXR agonist described herein to an individual in need thereof increases the secretion of enteroendocrine peptide products (e.g., GLP-1, GLP-2, PYY, oxytonmodulin or the like) from L-cells that line the gastrointestinal tract. In some embodiments, elevated levels of GLP-1 enhance healing of an injured pancreas. In some embodiments, an ASBTI and/or an enteroendocrine peptide enhancing agent and/or a FXR agonist described herein is administered in combination with a DPP-IV inhibitor. In some instances, inhibition of DPP-IV reduces the degradation of enteroendocrine peptide products (e.g. GLP-1) thereby prolonging the beneficial effects of the enteroendocrine peptide product.

In some embodiments of any of the methods described herein, administration of an ASBT inhibitor and/or an enteroendocrine peptide enhancing agent and/or a FXR agonist described herein increases the level of GLP-1 in the blood and/or plasma of an individual by from about 1.1 times to about 30 times compared to the level of GLP-1 in the blood and/or plasma of the individual prior to administration of the ASBTI and/or enteroendocrine peptide enhancing agent and/or FXR agonist. In some embodiments of any of the methods described herein, administration of the ASBTI and/or enteroendocrine peptide enhancing agent and/or FXR agonist described herein increases the level of GLP-1 in the blood and/or plasma of an individual by from about 1.1 times to about 20 times compared to the level of GLP-1 in the blood and/or plasma of the individual prior to administration of the ASBTI and/or enteroendocrine peptide enhancing agent and/or FXR agonist. In some embodiments of any of the methods described herein, administration of an ASBT inhibitor and/or an enteroendocrine peptide enhancing agent and/or a FXR agonist described herein increases the level of GLP-1 in the blood and/or plasma of an individual by from about 1.5 times to about 10 times compared to the level of GLP-1 in the blood and/or plasma of the individual prior to administration of the ASBTI and/or an enteroendocrine peptide enhancing agent and/or a FXR agonist. In some embodiments of any of the methods described herein, administration of an ASBT inhibitor and/or an enteroendocrine peptide enhancing agent and/or a FXR agonist described herein increases the level of GLP-1 in the blood and/or plasma of an individual by from about 2 times to about 8 times compared to the level of GLP-1 in the blood and/or plasma of the individual prior to administration of the ASBTI and/or an enteroendocrine peptide enhancing agent and/or a FXR agonist. In some embodiments of any of the methods described herein, administration of an ASBT inhibitor and/or an enteroendocrine peptide enhancing agent and/or a FXR agonist described herein increases the level of GLP-1 in the blood and/or plasma of an individual by from about 2 times to about 6 times compared to the level of GLP-1 in the blood and/or plasma of the individual prior to administration of the ASBTI and/or an enteroendocrine peptide enhancing agent and/or a FXR agonist.

In some instances, an increase in GLP-1 level of from about 2 times to about 3 times following the administration of an ASBT inhibitor and/or an enteroendocrine peptide enhancing agent and/or a FXR agonist described herein compared to the level of GLP-1 in the blood and/or plasma of the individual prior to administration of the ASBTI and/or an enteroendocrine peptide enhancing agent and/or a FXR agonist is associated with a healing effect in an inflamed pancreas.

Also provided herein is a method for treating conditions (e.g., pancreatitis) that are ameliorated by increased secretion of L-cell enteroendocrine peptides comprising contacting the gastrointestinal tract, including the distal ileum and/or the colon and/or the rectum, of an individual in need thereof with a therapeutically effective amount of any ASBTI compound and/or an enteroendocrine peptide enhancing agent and/or a FXR agonist described herein. L-cells are highly specialized gut enteroendocrine cells expressed along the gastrointestinal tract. The majority of L cells are located in the distal gastrointestinal tract, predominantly in the ileum and colon. The L-cells in the enteric endocrine system do not secrete their hormone continuously. Instead, they respond to changes in the environment within the lumen of the digestive tube, including changes in bile acid concentrations in the lumen of the digestive tube. The apical border of L-cells is in contact with the contents of the gastrointestinal lumen. Enteroendocrine peptides secreted by L-cells include GLP-1, GLP-2, PYY and oxyntomodulin. In certain instances, the methods described herein enhance L-cell secretion of one or more enteroendocrine hormones.

In some embodiments, the methods described herein enhance L-cell secretion of GLP-1, GLP-2, PYY or oxyntomodulin or combinations thereof. In certain embodiments, enhanced secretion of multiple enteroendocrine hormones (e.g., enhanced secretion of PYY and/or GLP-1 and/or GLP-2 and/or oxyntomodulin) is more effective for healing of an inflamed pancreas compared to enhanced secretion of any single enteroendocrine hormone. In certain embodiments, enhanced secretion of multiple enteroendocrine hormones (e.g., enhanced secretion of PYY and/or GLP-1 and/or GLP-2 and/or oxyntomodulin) is more effective for reducing symptoms of pancreatitis (e.g., pain) and/or duration of illness compared to enhanced secretion of any single enteroendocrine hormone.

In certain instances, contacting the distal ileum of an individual with an ASBTI (e.g., any ASBTI described herein) inhibits bile acid reuptake and increases the concentration of bile acids in the vicinity of L-cells in the distal ileum and/or colon and/or rectum, thereby reducing intraenterocyte bile acids, enhancing the release of enteroendocrine peptides, and/or reducing damage to pancreas caused by hyper-activation of pancreatic enzymes and/or auto-digestion of pancreas. Without being limited to any particular theory, bile acids and/or bile salts interact with TGR5 receptors on the apical surface of L-cells to trigger the release of one or more enteroendocrine hormones into systemic circulation and/or the gastrointestinal lumen. Under physiological conditions, the concentration of enteroendocrine hormones varies in the gastrointestinal tract. By way of example, in the absence of an ASBTI, PYY concentrations in the upper small intestine are about ˜5 μmol/g tissue, about ˜80 μmol/g tissue in the distal ileum and ascending colon, ˜200 μmol/g tissue in the sigmoid colon, and ˜500 μmol/g tissue in the rectum. In some embodiments, the administration of one or more ASBTIs, according to methods described herein, increases concentrations of one or more enteroendocrine peptides in the gastrointestinal lumen and/or systemic circulation compared to physiological concentrations of the enteroendocrine peptides in the absence of an ASBTI.

Administration of a compound described herein is achieved in any suitable manner including, by way of non-limiting example, by oral, enteric, parenteral (e.g., intravenous, subcutaneous, intramuscular), intranasal, buccal, topical, rectal, or transdermal administration routes. Any compound or composition described herein is administered in a method or formulation appropriate to treat a new born or an infant. Any compound or composition described herein is administered in an oral formulation (e.g., solid or liquid) to treat a new born or an infant. Any compound or composition described herein is administered prior to ingestion of food, with food or after ingestion of food.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein are administered to an individual susceptible to or otherwise at risk of a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain instances, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain embodiments, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In some embodiments, doses administered include those up to the maximum tolerable dose by a newborn or an infant.

In certain embodiments, about 0.001-5000 mg per day, from about 0.001-1500 mg per day, about 0.001 to about 100 mg/day, about 0.001 to about 50 mg/day, or about 0.001 to about 30 mg/day, or about 0.001 to about 10 mg/day of a compound described herein is administered to an individual in need thereof. In various embodiments, the desired dose is conveniently presented in a single dose or in divided doses administered simultaneously (or over a short period of time) or at appropriate intervals, for example as two, three, four or more sub-doses per day. In various embodiments, a single dose is from about 0.001 mg/kg to about 500 mg/kg. In various embodiments, a single dose is from about 0.001, 0.01, 0.1, 1, or 10 mg/kg to about 10, 50, 100, or 250 mg/kg. In various embodiments, a single dose of an ASBTI is from about 0.001 mg/kg to about 100 mg/kg. In various embodiments, a single dose of an ASBTI is from about 0.001 mg/kg to about 50 mg/kg. In various embodiments, a single dose of an ASBTI is from about 0.001 mg/kg to about 10 mg/kg. In various embodiments, a single dose of an ASBTI is administered every 6 hours, every 12 hours, every 24 hours, every 48 hours, every 72 hours, every 96 hours, every 5 days, every 6 days, or once a week. In some embodiments the total single dose of an ASBTI and/or an enteroendocrine peptide enhancing agent and/or a FXR agonist is in the range described above.

In the case wherein the patient's status does improve, upon the doctor's discretion an ASBTI and/or an enteroendocrine peptide enhancing agent and/or a FXR agonist is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%. In some embodiments the total single dose of an ASBTI and/or an enteroendocrine peptide enhancing agent and/or a FXR agonist is in the range described above.

Once improvement of the patient's conditions has occurred (e.g., weight loss), a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms (e.g., weight gain).

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are optionally determined by pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. In certain embodiments, data obtained from cell culture assays and animal studies are used in formulating a range of dosage for use in human. In specific embodiments, the dosage of compounds described herein lies within a range of circulating concentrations that include the ED₅₀ with minimal toxicity. The dosage optionally varies within this range depending upon the dosage form employed and the route of administration utilized.

In some embodiments, the systemic exposure of a therapeutically effective amount of any non-systemic ASBTI described herein (e.g., an ASBTI that comprises a group L-K) is reduced when compared to the systemic exposure of a therapeutically effective amount of any systemically absorbed ASBTI (e.g. Compounds 100A, 100C). In some embodiments, the AUC of a therapeutically effective amount of any non-systemic ASBTI described herein (e.g., an ASBTI that comprises a group L-K) is at least 10%, at least 20%, at least 30%, at least 40%, at least 50%, at least 60%, at least 70%, at least 80% or at least 90% reduced when compared to the AUC of any systemically absorbed ASBTI (e.g. Compounds 100A, 100C).

In some embodiments, the systemic exposure of a therapeutically effective amount of a compound of Formula I that is not systemically absorbed (e.g., a compound of Formula I that comprises a group L-K) is reduced when compared to the systemic exposure of a therapeutically effective amount of Compound 100A. In some embodiments, the AUC of a therapeutically effective amount of a compound of Formula I that is not systemically absorbed (e.g., a compound of Formula I that comprises a group L-K) is about 10%, about 20%, about 30%, about 40%, about 50%, about 60%, about 70%, about 80% or about 90% reduced when compared to the AUC of a therapeutically effective amount of Compound 100A. In some embodiments, the AUC of a therapeutically effective amount of a compound of Formula I that is not systemically absorbed (e.g., a compound of Formula I that comprises a group L-K) is about 50% reduced when compared to the AUC of a therapeutically effective amount of Compound 100A. In other embodiments, the AUC of a therapeutically effective amount of a compound of Formula I that is not systemically absorbed (e.g., a compound of Formula I that comprises a group L-K) is about 75% reduced when compared to the AUC of a therapeutically effective amount of Compound 100A.

In some embodiments, the systemic exposure of a therapeutically effective amount of a compound of Formula II that is not systemically absorbed (e.g., a compound of Formula II that comprises a group L-K) is reduced when compared to the systemic exposure of a therapeutically effective amount of Compound 100A. In some embodiments, the AUC of a therapeutically effective amount of a compound of Formula II that is not systemically absorbed (e.g., a compound of Formula II that comprises a group L-K) is about 10%, about 20%, about 30%, about 40%, about 50%, about 60%, about 70%, about 80% or about 90% reduced when compared to the AUC of a therapeutically effective amount of Compound 100A. In some embodiments, the AUC of a therapeutically effective amount of a compound of Formula II that is not systemically absorbed (e.g., a compound of Formula II that comprises a group L-K) is about 50% reduced when compared to the AUC of a therapeutically effective amount of Compound 100A. In other embodiments, the AUC of a therapeutically effective amount of a compound of Formula II that is not systemically absorbed (e.g., a compound of Formula II that comprises a group L-K) is about 75% reduced when compared to the AUC of a therapeutically effective amount of Compound 100A.

In some embodiments, the systemic exposure of a therapeutically effective amount of a compound of Formula III, IIIA, IIIB or IIIC is reduced when compared to the systemic exposure of a therapeutically effective amount of Compound 100C. In some embodiments, the AUC of a therapeutically effective amount of a compound of Formula III, IIIA, IIIB or IIIC is about 10%, about 20%, about 30%, about 40%, about 50%, about 60%, about 70%, about 80% or about 90% reduced when compared to the AUC of a therapeutically effective amount of Compound 100C. In some embodiments, the AUC of a therapeutically effective amount of a compound of Formula III, IIIA, IIIB or IIIC is about 50% reduced when compared to the AUC of a therapeutically effective amount of Compound 100C. In other embodiments, the AUC of a therapeutically effective amount of a compound of Formula III, IIIA, IIIB or IIIC is about 75% reduced when compared to the AUC of a therapeutically effective amount of Compound 100C.

In some embodiments, the systemic exposure of a therapeutically effective amount of a compound of Formula IV that is not systemically absorbed (e.g., a compound of Formula IV that comprises a group L-K) is reduced when compared to the systemic exposure of a therapeutically effective amount of Compound 100A. In some embodiments, the AUC of a therapeutically effective amount of a compound of Formula IV that is not systemically absorbed (e.g., a compound of Formula I that comprises a group L-K) is about 10%, about 20%, about 30%, about 40%, about 50%, about 60%, about 70%, about 80% or about 90% reduced when compared to the AUC of a therapeutically effective amount of Compound 100A. In some embodiments, the AUC of a therapeutically effective amount of a compound of Formula IV that is not systemically absorbed (e.g., a compound of Formula IV that comprises a group L-K) is about 50% reduced when compared to the AUC of a therapeutically effective amount of Compound 100A. In other embodiments, the AUC of a therapeutically effective amount of a compound of Formula IV that is not systemically absorbed (e.g., a compound of Formula IV that comprises a group L-K) is about 75% reduced when compared to the AUC of a therapeutically effective amount of Compound 100A.

In some embodiments, the systemic exposure of a therapeutically effective amount of a compound of Formula V that is not systemically absorbed (e.g., a compound of Formula V that comprises a group L-K) is reduced when compared to the systemic exposure of a therapeutically effective amount of Compound 100A. In some embodiments, the AUC of a therapeutically effective amount of a compound of Formula V that is not systemically absorbed (e.g., a compound of Formula V that comprises a group L-K) is about 10%, about 20%, about 30%, about 40%, about 50%, about 60%, about 70%, about 80% or about 90% reduced when compared to the AUC of a therapeutically effective amount of Compound 100A. In some embodiments, the AUC of a therapeutically effective amount of a compound of Formula I that is not systemically absorbed (e.g., a compound of Formula V that comprises a group L-K) is about 50% reduced when compared to the AUC of a therapeutically effective amount of Compound 100A. In other embodiments, the AUC of a therapeutically effective amount of a compound of Formula I that is not systemically absorbed (e.g., a compound of Formula V that comprises a group L-K) is about 75% reduced when compared to the AUC of a therapeutically effective amount of Compound 100A.

In some embodiments, the systemic exposure of a therapeutically effective amount of a compound of Formula VI or VID that is not systemically absorbed (e.g., a compound of Formula VI or VID that comprises a group L-K) is reduced when compared to the systemic exposure of a therapeutically effective amount of Compound 100A. In some embodiments, the AUC of a therapeutically effective amount of a compound of Formula VI or VID that is not systemically absorbed (e.g., a compound of Formula VI or VID that comprises a group L-K) is about 10%, about 20%, about 30%, about 40%, about 50%, about 60%, about 70%, about 80% or about 90% reduced when compared to the AUC of a therapeutically effective amount of Compound 100A. In some embodiments, the AUC of a therapeutically effective amount of a compound of Formula VI or VID that is not systemically absorbed (e.g., a compound of Formula VI or VID that comprises a group L-K) is about 50% reduced when compared to the AUC of a therapeutically effective amount of Compound 100A. In other embodiments, the AUC of a therapeutically effective amount of a compound of Formula I that is not systemically absorbed (e.g., a compound of Formula VI or VID that comprises a group L-K) is about 75% reduced when compared to the AUC of a therapeutically effective amount of Compound 100A.

In certain embodiments, the Cmax of a therapeutically effective amount of any non-systemic ASBTI described herein (e.g., an ASBTI that comprises a group L-K) is at least 10%, at least 20%, at least 30%, at least 40%, at least 50%, at least 60%, at least 70%, at least 80% or at least 90% reduced when compared to the Cmax of any systemically absorbed ASBTI (e.g. Compound 100A).

By way of example, the Cmax of a therapeutically effective amount of a compound of Formula III, IIIA, IIIB or IIIC is about 10%, about 20%, about 30%, about 40%, about 50%, about 60%, about 70%, about 80% or about 90% reduced when compared to the Cmax of a therapeutically effective amount of Compound 100C. In some embodiments, the Cmax of a therapeutically effective amount of a compound of Formula III, IIIA, IIIB or IIIC is about 25% reduced when compared to the Cmax of a therapeutically effective amount of Compound 100C. In certain embodiments, the Cmax of a therapeutically effective amount of a compound of III, IIIA or 111B is about 50% reduced when compared to the Cmax of a therapeutically effective amount of Compound 100C. In other embodiments, the Cmax of a therapeutically effective amount of a compound of Formula III, IIIA, IIIB or IIIC is about 75% reduced when compared to the Cmax of a therapeutically effective amount of Compound 100C.

In certain embodiments, the pharmaceutical composition administered includes a therapeutically effective amount of an enteroendocrine peptide secretion enhancing agent, an absorption inhibitor and a carrier (e.g., an orally suitable carrier or a rectally suitable carrier, depending on the mode of intended administration). In certain embodiments, the pharmaceutical composition used or administered comprises an enteroendocrine peptide secretion enhancing agent, an absorption inhibitor, a carrier, and one or more of a cholesterol absorption inhibitor, an enteroendocrine peptide, a peptidase inhibitor, a spreading agent, and a wetting agent.

In a specific embodiment, the pharmaceutical composition used to prepare a rectal dosage form or administered rectally comprises an enteroendocrine peptide secretion enhancing agent, an absorption inhibitor, a rectally suitable carrier, an optional cholesterol absorption inhibitor, an optional enteroendocrine peptide, an optional peptidase inhibitor, an optional spreading agent, and an optional wetting agent. In certain embodiments, rectally administered compositions evokes an anorectal response. In specific embodiments, the anorectal response is an increase in secretion of one or more enteroendocrine by cells (e.g., L-cells) in the colon and/or rectum (e.g., in the epithelial layer of the colon and/or rectum). In some embodiments, the anorectal response persists for at least 1, 2, 3, 4, 5 ,6, 7, 8, 9, 10, 11, 12, 13, 14, 15, 16, 17, 18, 19, 20, 21, 22, 23 or 24 hours. In other embodiments the anorectal response persists for a period between 24 hours and 48 hours, while in other embodiments the anorectal response persists for persists for a period greater than 48 hours.

In another specific embodiment, the pharmaceutical composition used to prepare an oral dosage form or administered orally comprises an enteroendocrine peptide secretion enhancing agent, an absorption inhibitor, an orally suitable carrier, an optional cholesterol absorption inhibitor, an optional enteroendocrine peptide, an optional peptidase inhibitor, an optional spreading agent, and an optional wetting agent. In certain embodiments, the orally administered compositions evokes an anorectal response. In specific embodiments, the anorectal response is an increase in secretion of one or more enteroendocrine by cells in the colon and/or rectum (e.g., in L-cells the epithelial layer of the colon and/or rectum). In some embodiments, the anorectal response persists for at least 1, 2, 3, 4, 5, 6, 7, 8, 9, 10, 11, 12, 13, 14, 15, 16, 17, 18, 19, 20, 21, 22, 23 or 24 hours. In other embodiments the anorectal response persists for a period between 24 hours and 48 hours, while in other embodiments the anorectal response persists for persists for a period greater than 48 hours.

### Routes of Administration and Dosage

In some embodiments, the compositions described herein and the compositions administered in the methods described herein are formulated to enhance enteroendocrine peptide secretion and to evoke an anorectal response. In certain embodiments, the compositions described herein are formulated for rectal or oral administration. In some embodiments, such formulations are administered rectally or orally, respectively. In some embodiments, the compositions described herein are combined with a device for local delivery of the compositions to the rectum and/or colon (sigmoid colon, transverse colon, or ascending colon). In certain embodiments, for rectal administration the composition described herein are formulated as enemas, rectal gels, rectal foams, rectal aerosols, suppositories, jelly suppositories, or retention enemas. In some embodiments, for oral administration the compositions described herein are formulated for oral administration and enteric delivery to the colon.

In certain embodiments, the compositions or methods described herein are non-systemic. In some embodiments, compositions described herein deliver the enteroendocrine peptide secretion enhancing agent to the distal ileum, colon, and/or rectum and not systemically (e.g., a substantial portion of the enteroendocrine peptide secretion enhancing agent is not systemically absorbed). In some embodiments, oral compositions described herein deliver the enteroendocrine peptide secretion enhancing agent to the distal ileum, colon, and/or rectum and not systemically (e.g., a substantial portion of the enteroendocrine peptide secretion enhancing agent is not systemically absorbed). In some embodiments, rectal compositions described herein deliver the enteroendocrine peptide secretion enhancing agent to the distal ileum, colon, and/or rectum and not systemically (e.g., a substantial portion of the enteroendocrine peptide secretion enhancing agent is not systemically absorbed). In certain embodiments, non-systemic compositions described herein deliver less than 90% w/w of the enteroendocrine peptide secretion enhancing agent systemically. In certain embodiments, non-systemic compositions described herein deliver less than 80% w/w of the enteroendocrine peptide secretion enhancing agent systemically. In certain embodiments, non-systemic compositions described herein deliver less than 70% w/w of the enteroendocrine peptide secretion enhancing agent systemically. In certain embodiments, non-systemic compositions described herein deliver less than 60% w/w of the enteroendocrine peptide secretion enhancing agent systemically. In certain embodiments, non-systemic compositions described herein deliver less than 50% w/w of the enteroendocrine peptide secretion enhancing agent systemically. In certain embodiments, non-systemic compositions described herein deliver less than 40% w/w of the enteroendocrine peptide secretion enhancing agent systemically. In certain embodiments, non-systemic compositions described herein deliver less than 30% w/w of the enteroendocrine peptide secretion enhancing agent systemically. In certain embodiments, non-systemic compositions described herein deliver less than 25% w/w of the enteroendocrine peptide secretion enhancing agent systemically. In certain embodiments, non-systemic compositions described herein deliver less than 20% w/w of the enteroendocrine peptide secretion enhancing agent systemically. In certain embodiments, non-systemic compositions described herein deliver less than 15% w/w of the enteroendocrine peptide secretion enhancing agent systemically. In certain embodiments, non-systemic compositions described herein deliver less than 10% w/w of the enteroendocrine peptide secretion enhancing agent systemically. In certain embodiments, non-systemic compositions described herein deliver less than 5% w/w of the enteroendocrine peptide secretion enhancing agent systemically. In some embodiments, systemic absorption is determined in any suitable manner, including the total circulating amount, the amount cleared after administration, or the like.

In certain embodiments, the compositions and/or formulations described herein are administered at least once a day. In certain embodiments, the formulations containing the enteroendocrine peptide secretion enhancing agents are administered at least twice a day, while in other embodiments the formulations containing the enteroendocrine peptide secretion enhancing agents are administered at least three times a day. In certain embodiments, the formulations containing the enteroendocrine peptide secretion enhancing agents are administered up to five times a day. It is to be understood that in certain embodiments, the dosage regimen of composition containing the enteroendocrine peptide secretion enhancing agents described herein to is determined by considering various factors such as the patient's age, sex, and diet.

The concentration of the enteroendocrine peptide secretion enhancing agents administered in the formulations described herein ranges from about 1 mM to about 1 M. In certain embodiments the concentration of the enteroendocrine peptide secretion enhancing agents administered in the formulations described herein ranges from about 1 mM to about 750 mM. In certain embodiments the concentration of the enteroendocrine peptide secretion enhancing agents administered in the formulations described herein ranges from about 1 mM to about 500 mM. In certain embodiments the concentration of the enteroendocrine peptide secretion enhancing agents administered in the formulations described herein ranges from about 5 mM to about 500 mM. In certain embodiments the concentration of the enteroendocrine peptide secretion enhancing agents administered in the formulations described herein ranges from about 10 mM to about 500 mM. In certain embodiments the concentration of the enteroendocrine peptide secretion enhancing agents administered in the formulations described herein ranges from about 25 mM to about 500 mM. In certain embodiments the concentration of the enteroendocrine peptide secretion enhancing agents administered in the formulations described herein ranges from about 50 mM to about 500 mM. In certain embodiments the concentration of the enteroendocrine peptide secretion enhancing agents administered in the formulations described herein ranges from about 100 mM to about 500 mM. In certain embodiments the concentration of the enteroendocrine peptide secretion enhancing agents administered in the formulations described herein ranges from about 200 mM to about 500 mM.

In certain embodiments, any composition described herein comprises a therapeutically effective amount (e.g., to treat pancreatitis) of an enteroendocrine peptide secretion enhancing agent (e.g., bile acid). In some embodiments, compositions described herein comprise or methods described herein comprise administering about 0.01 mg to about 10 g of an enteroendocrine peptide secretion enhancing agent (e.g., bile acid). In certain embodiments, a composition described herein comprises or a method described herein comprises administering about 0.1 mg to about 500 mg of an enteroendocrine peptide secretion enhancing agent (e.g., bile acid). In certain embodiments, a composition described herein comprises or a method described herein comprises administering about 0.1 mg to about 100 mg of an enteroendocrine peptide secretion enhancing agent (e.g., bile acid). In certain embodiments, a composition described herein comprises or a method described herein comprises administering about 0.1 mg to about 50 mg of an enteroendocrine peptide secretion enhancing agent (e.g., bile acid). In certain embodiments, a composition described herein comprises or a method described herein comprises administering about 0.1 mg to about 10 mg of an enteroendocrine peptide secretion enhancing agent (e.g., bile acid). In certain embodiments, a composition described herein comprises or a method described herein comprises administering about 0.5 mg to about 10 mg of an enteroendocrine peptide secretion enhancing agent (e.g., bile acid). In some embodiments, compositions described herein comprise or methods described herein comprise administering about 0.1 mmol to about 1 mol of an enteroendocrine peptide secretion enhancing agent (e.g., bile acid). In certain embodiments, a composition described herein comprises or a method described herein comprises administering about 0.01 mmol to about 500 mmol of an enteroendocrine peptide secretion enhancing agent (e.g., bile acid). In certain embodiments, a composition described herein comprises or a method described herein comprises administering about 0.1 mmol to about 100 mmol of an enteroendocrine peptide secretion enhancing agent (e.g., bile acid). In certain embodiments, a composition described herein comprises or a method described herein comprises administering about 0.5 mmol to about 30 mmol of an enteroendocrine peptide secretion enhancing agent (e.g., bile acid). In certain embodiments, a composition described herein comprises or a method described herein comprises administering about 0.5 mmol to about 20 mmol of an enteroendocrine peptide secretion enhancing agent (e.g., bile acid). In certain embodiments, a composition described herein comprises or a method described herein comprises administering about 1 mmol to about 10 mmol of an enteroendocrine peptide secretion enhancing agent (e.g., bile acid). In certain embodiments, a composition described herein comprises or a method described herein comprises administering about 0.01 mmol to about 5 mmol of an enteroendocrine peptide secretion enhancing agent (e.g., bile acid). In certain embodiments, a composition described herein comprises or a method described herein comprises administering about 0.1 mmol to about 1 mmol of an enteroendocrine peptide secretion enhancing agent (e.g., bile acid). In various embodiments, certain enteroendocrine peptide secretion enhancing agents (e.g., bile acids) have different potencies and dosing is optionally adjusted accordingly. For example, the investigation in TGR5-transfected CHO cells of TGR5 agonist potency of natural bile acids indicates the following rank of potency: Lithocholic acid (LCA)>deoxycholic acid (DCA)>murocholic acid (Muro-CA)>lagodeoxycholic acid (lago-DCA)>chenodeoxycholic (CDCA)>cholic acid (CA)>hyodeoxycholic acid (HDCA>ursodeoxycholic acid (UDCA); and assays on TGR5-transfected CHO cells demonstrate that EC₅₀ (in M) for UDCA was 36.4, TauroCA (TCA) 4.95 and LCA 0.58.

In certain embodiments, by targeting the distal gastrointestinal tract (e.g., distal ileum, colon, and/or rectum), compositions and methods described herein provide efficacy (e.g., in reducing inflammatory cytokines) with a reduced dose of enteroendocrine peptide secretion enhancing agent (e.g., as compared to an oral dose that does not target the distal gastrointestinal tract).

### Rectal Administration Formulations

The pharmaceutical compositions described herein for the non-systemic delivery of enteroendocrine peptide secretion enhancing agents to the rectum and/or colon are formulated for rectal administration as rectal enemas, rectal foams, rectal gels, and rectal suppositories. The components of such formulations are described herein. It is to be understood that as used herein, pharmaceutical compositions and compositions are or comprise the formulations as described herein.

### Rectal Enemas

In certain embodiments, the compositions described herein are formulated as rectal enema formulations for non-systemic delivery of enteroendocrine peptide secretion enhancing agents. In certain embodiments, such rectal enemas are formulated as a solution, aqueous suspension or emulsion. In some embodiments, solution enemas contain a carrier vehicle, an enteroendocrine peptide secretion enhancing agent, an absorption inhibitor (e.g., of the enteroendocrine peptide secretion enhancing agent across the rectal or colonic mucosa), and one or more of the following: a solubilizer, a preservative, a chelating agent, a buffer for pH regulation, and a thickener. In certain embodiments, rectal enemas are formulated as an emulsion or aqueous suspension containing a carrier vehicle, at least one enteroendocrine peptide secretion enhancing agent, at least one agent for inhibiting absorption of the enteroendocrine peptide secretion enhancing agent across the rectal or colonic mucosa, and one or more of the following: a preservative, a chelating agent, a buffer for pH regulation, a solubilizer, a thickener, and an emulsifier/surfactant.

In certain embodiments, rectal enemas are formulated such that a enteroendocrine peptide secretion enhancing agent is dissolved or dispersed in a suitable flowable carrier vehicle, including but not limited to water, alcohol or an aqueous-alcoholic mixture. In certain embodiments, the carrier vehicle is thickened with natural or synthetic thickeners. In further embodiments the rectal enema formulations also contain a lubricant.

In some embodiments, unit dosages of such enema formulations are administered from prefilled bags or syringes.

In certain embodiments, the volume of enema administered using such rectal enema formulations is a volume suitable for achieving a desired result, e.g., from about 10 mL to about 1000 mL. In certain embodiments, the volume of enema administered using such rectal enema formulations is from about 10 mL to about 900 mL. In certain embodiments, the volume of enema administered using such rectal enema formulations is from about 10 mL to about 800 mL. In certain embodiments, the volume of enema administered using such rectal enema formulations is from about 10 mL to about 700 mL. In certain embodiments, the volume of enema administered using such rectal enema formulations is from about 10 mL to about 600 mL. In certain embodiments, the volume of enema administered using such rectal enema formulations is from about 10 mL to about 500 mL. In certain embodiments, the volume of enema administered using such rectal enema formulations is from about 10 mL to about 400 mL. In certain embodiments, the volume of enema administered using such rectal enema formulations is from about 10 mL to about 300 mL. In certain embodiments, the volume of enema administered using such rectal enema formulations is from about 10 mL to about 200 mL. In certain embodiments, the volume of enema administered using such rectal enema formulations is from about 10 mL to about 100 mL. In some embodiments, such enemas may have a volume of less than 1 L, less than 900 mL, less than 700 mL, less than 600 mL, less than 500 mL, less than 250 mL, less than 100 mL, less than 30 mL, less than 10 mL, less than 3 mL, or the like.

### Rectal Foams

In certain instances, leakage is a problem associated with enemas. As such, it is often desirable or necessary for patients to lie down during administration of enemas. In some embodiments, rectal administration using foams overcomes the problem of leakage from the rectum following administration.

In certain embodiments, the pharmaceutical compositions are formulated as rectal foams. In some embodiments, rectal foams are used for the rectal administration and for local or non-systemic delivery of enteroendocrine peptide secretion enhancing agents to the rectum and/or colon. Such rectal foams formulations contain an enteroendocrine peptide secretion enhancing agent dissolved or suspended in a liquid carrier vehicle, an absorption inhibitor (e.g., of the enteroendocrine peptide secretion enhancing agent across the rectal or colonic mucosa), a surfactant/emulsifier with foaming properties and a propellant (e.g., a propellant gas). In certain embodiments, rectal foam formulations also contain one or more of the following: a suspending/solubilizing agent, a thickener, a preservative, a chelating agent, a buffer, an antioxidant, a tonicity modifiers, and a spreading agent. In certain embodiments, surfactants/emulsifiers include, by way of non-limiting example, non-ionic surfactants, anionic surfactants, cationic surfactants, and combinations thereof.

In certain embodiments, rectal foam formulations are filled in pressurized containers prior to rectal administration. In certain embodiments the pressurized container is a can. In certain embodiments, propellants used herein include, by way of non-limiting example, hydrocarbons (such as isobutane, N-butane or propane), fluorocarbons (e.g. dichlorodifluoromethane and dichlorotetrafluoroethane), chlorofluorocarbons, dimethyl ether, hydrofluorocarbons, compressed gases, freon (such as freon 12, freon 114), hydrochlorofluorocarbons, hydrofluorocarbons or mixtures thereof.

In some embodiments, the maximum amount of propellant used is determined by its miscibility with other components in the composition to form a mixture, such as a homogeneous mixture. In certain embodiments, the minimal level of propellant used in the composition is determined by the desired foam characteristics, and its ability to substantially or completely evacuate the container.

In some embodiments, the propellant concentration used in such rectal foam formulations is about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 50%, 55% to about 60% (w/w).

In certain embodiments, rectal foams are formed upon rectal administration, wherein the dispensing valve of the can allows rapid expansion of the propellant, triggering the foaming action of the surfactant and resulting foam forms within the rectum and colon. In other embodiments, the rectal foams used for rectal administration of the compositions described herein are formed within the dispensing container prior to rectal administration.

The distance the foam can reach within the colon and rectum is controlled by controlling the foam propelling properties by varying the type and quantity of propellant used. The volume of foam administered using such rectal foam formulations is from about 10 mL to about 1000 mL. In certain embodiments, the volume of a composition described herein (e.g., a foam) described herein or used in a method described herein (e.g., a foam, enema, or gel) is from about 10 mL to about 900 mL. In certain embodiments, the volume of a composition described herein (e.g., a foam) described herein or used in a method described herein (e.g., a foam, enema, or gel) is from about 10 mL to about 800 mL. In certain embodiments, the volume of a composition described herein (e.g., a foam) described herein or used in a method described herein (e.g., a foam, enema, or gel) is from about 10 mL to about 700 mL. In certain embodiments, the volume of a composition described herein (e.g., a foam) described herein or used in a method described herein (e.g., a foam, enema, or gel) is from about 10 mL to about 600 mL. In certain embodiments, the volume of a composition described herein (e.g., a foam) described herein or used in a method described herein (e.g., a foam, enema, or gel) is from about 10 mL to about 500 mL. In certain embodiments, the volume of a composition described herein (e.g., a foam) described herein or used in a method described herein (e.g., a foam, enema, or gel) is from about 10 mL to about 400 mL. In certain embodiments, the volume of a composition described herein (e.g., a foam) described herein or used in a method described herein (e.g., a foam, enema, or gel) is from about 10 mL to about 300 mL. In certain embodiments, the volume of a composition described herein (e.g., a foam) described herein or used in a method described herein (e.g., a foam, enema, or gel) is from about 10 mL to about 200 mL. In certain embodiments, the volume of a composition described herein (e.g., a foam) described herein or used in a method described herein (e.g., a foam, enema, or gel) is from about 10 mL to about 100 mL. In specific embodiments, the volume of a composition described herein (e.g., a foam) described herein or used in a method described herein (e.g., a foam, enema, or gel) is about 20 mL to about 60 mL, about 20 mL, about 40 mL, or about 60 mL.

### Rectal Gels

In some embodiments, the pharmaceutical compositions described herein are formulated as rectal gels. In certain embodiments, the rectal gels are suitable for the regional or local non-systemic administration of one or more enteroendocrine peptide secretion enhancing agents to the rectum and/or colon. In some embodiments, rectal gel formulations contain at least one enteroendocrine peptide secretion enhancing agent dissolved or suspended in a solvent/liquid carrier vehicle, an absorption inhibitor (e.g., of the enteroendocrine peptide secretion enhancing agent across the rectal or colonic mucosa) and at least one thickening agents. In certain embodiments such rectal gel formulations also contain one or more of the following: a buffering agent(s), a preservative(s), and an antioxidant(s).

In certain embodiments, rectal gels have gel-like consistencies but are sufficiently flowable so as to be capable of local or regional administration through a catheter, needle, syringe, or other comparable means of local or regional administration.

In some embodiments, the concentration of a thickener used in a rectal gel formulation is in an amount or concentration suitable to achieve a desired thickness or viscosity, e.g., from about 0.05% to about 10% by weight. In certain embodiments, the concentration of the thickener used in such rectal gel formulations ranges from about 0.05% to about 8% by weight. In certain embodiments, the concentration of the thickener used in such rectal gel formulations ranges from about 0.05% to about 7% by weight. In certain embodiments, the concentration of the thickener used in such rectal gel formulations ranges from about 0.05% to about 6% by weight. In certain embodiments, the concentration of the thickener used in such rectal gel formulations ranges from about 0.05% to about 5% by weight. In certain embodiments, the concentration of the thickener used in such rectal gel formulations ranges from about 0.05% to about 4% by weight. In certain embodiments, the concentration of the thickener used in such rectal gel formulations ranges from about 0.05% to about 3% by weight. In certain embodiments, the concentration of the thickener used in such rectal gel formulations ranges from about 0.05% to about 2% by weight. In certain embodiments, the concentration of the thickener used in such rectal gel formulations ranges from about 0.05% to about 1% by weight. In certain embodiments the rectal gel formulation includes methyl cellulose having a concentration from about 0.05% to about 2%, while in other embodiments the rectal gel formulation includes methyl cellulose having a concentration of about 1%.

In some embodiments, the any formulation described herein (e.g., arectal gel formulation) has a viscosity ranging from about 500 to about 50,000 centipoise (cP) at 25 C. In certain embodiments, the viscosity of the formulation described herein is from about 500 to about 40,000 centipoise (cP) at 25 C. In certain embodiments, the viscosity of the formulation described herein is from about 500 to about 30,000 centipoise (cP) at 25 C. In certain embodiments, the viscosity of the formulation described herein is from about 500 to about 20,000 centipoise (cP) at 25 C. In certain embodiments, the viscosity of the formulation described herein is from about 500 to about 10,000 centipoise (cP) at 25 C. In some embodiments, the formulation has a final viscosity of less than about 40,000 centipoises (cP), 20,000 cP, 15,000 cP, or 10,000 cP at 25 C. In some embodiments, the formulation has a viscosity of about 5,000 cP, 6,000 cP, 7,000 cP, 8,000 cP, 9,000 cP, 10,000 cP, 12,000 cP, 15,000 cP, 18,000 cP, 20,000 cP, 25,000 cP, 30,000 cP, 35,000 cP, or 40,000 cP at 25 C. In some embodiments, the formulation has a viscosity of about 1,000-20,000 cP, 5,000-15,000 cP, 6,000-12,000 cP, 7,000-10,000,500-3500 cP, 500-300cP, 1,000-2,000 cP, or about 1,500 cP at 25° C. In specific embodiments, the formulation has a viscosity of 1,000 cP to about 2,500 cP, or about 1,500 cP at 25 C. In certain embodiments, the amount of thickener used in a composition described herein is sufficient to achieve a viscosity as described herein.

In some embodiments, unit dosages of such rectal gel formulations are administered from prefilled bags or syringes.

### Rectal Suppositories

In some embodiments, the pharmaceutical compositions described herein are also formulated as a suppository. In certain embodiments, suppositories are formulated for the regional or local non-systemic administration of one or more enteroendocrine peptide secretion enhancing agents to the rectum and/or colon.

In some embodiments, rectal suppository formulations contain a enteroendocrine peptide secretion enhancing agent, an absorption inhibitor (e.g., of the enteroendocrine peptide secretion enhancing agent across the rectal or colonic mucosa) and at least one pharmaceutically acceptable suppository base. In some embodiments, suppository formulation are prepared by combining an enteroendocrine peptide secretion enhancing agent with a pharmaceutically acceptable suppository base, melted, poured into a mould or moulds and cooled.

In certain embodiments, pharmaceutically acceptable suppository bases include, by way of non-limiting example, cocoa butter, beeswax, esterified fatty acids, glycerinated gelatin, semisynthetic glycerides of vegetable saturated fatty acids, polyethylene glycols, Witepsol, and polyoxyethylene sorbitan fatty acid esters.

In certain embodiments, the suppository formulations used to deliver one or more enteroendocrine peptide secretion enhancing agents to the rectum and/or colon also contain one or more of the following: buffering agents, preservatives, antioxidants, surfactants, and thickeners.

In some embodiments, suppositories contain from 0.5 to 10 mg of an enteroendocrine peptide secretion enhancing agent. In specific embodiments, suppositories contain from 1 to 5 mg of an enteroendocrine peptide secretion enhancing agent.

### Components Used in Rectal Delivery/Administration Formulations

In certain embodiments, liquid carrier vehicles in the compositions and/or formulations described herein include, by way of non-limiting example, purified water, propylene glycol, polyethyleneglycol, ethanol, 1-propanol, 2-propanol, 1-propen-3-ol (allyl alcohol), propylene glycol, glycerol, 2-methyl-2-propanol, formamide, methyl formamide, dimethyl formamide, ethyl formamide, diethyl formamide, acetamide, methyl acetamide, dimethyl acetamide, ethyl acetamide, diethyl acetamide, 2-pyrrolidone, N-methyl-2-pyrrolidone, N-ethyl-2-pyrrolidone, tetramethyl urea, 1,3-dimethyl-2-imidazolidinone, propylene carbonate, 1,2-butylene carbonate, 2,3-butylene carbonate, dimethyl sulfoxide, diethyl sulfoxide, hexamethyl phosphoramide, pyruvic aldehyde dimethylacetal, dimethylisosorbide and combinations thereof.

In some embodiments, stabilizers used in compositions and/or formulations described herein include, but are not limited to, partial glycerides of polyoxyethylenic saturated fatty acids.

In certain embodiments, surfactants/emulsifiers used in the compositions and/or formulations described herein include, by way of non-limiting example, mixtures of cetostearylic alcohol with sorbitan esterified with polyoxyethylenic fatty acids, polyoxyethylene fatty ethers, polyoxyethylene fatty esters, fatty acids, sulfated fatty acids, phosphated fatty acids, sulfosuccinates, amphoteric surfactants, non-ionic poloxamers, non-ionic meroxapols, petroleum derivatives, aliphatic amines, polysiloxane derivatives, sorbitan fatty acid esters, laureth-4, PEG-2 dilaurate, stearic acid, sodium lauryl sulfate, dioctyl sodium sulfosuccinate, cocoamphopropionate, poloxamer 188, meroxapol 258, triethanolamine, dimethicone, polysorbate 60, sorbitan monostearate, pharmaceutically acceptable salts thereof, and combinations thereof.

In some embodiments, non-ionic surfactants used in compositions and/or formulations described herein include, by way of non-limiting example, phospholipids, alkyl poly(ethylene oxide), poloxamers, polysorbates, sodium dioctyl sulfosuccinate, Brij™-30 (Laureth-4), Brij™-58 (Ceteth-20) and Brij™-78 (Steareth-20), Brij™-721 (Steareth-21), Crillet-1 (Polysorbate 20), Crillet-2 (Polysorbate 40), Crillet-3 (Polysorbate 60), Crillet 45 (Polysorbate 80), Myrj-52 (PEG-40 Stearate), Myrj-53 (PEG-50 Stearate), Pluronic™F77 (Poloxamer 217), Pluronic™F87 (Poloxamer 237), Pluronic™F98 (Poloxamer 288), Pluronic™L62 (Poloxamer 182), Pluronic™L64 (Poloxamer 184), Pluronic™F68 (Poloxamer 188), Pluronic™L81 (Poloxamer 231), Pluronic™L92 (Poloxamer 282), Pluronic™L101 (Poloxamer 331), Pluronic™P103 (Poloxamer 333), Pluracare™F 108 NF (Poloxamer 338), and Pluracare™F 127 NF (Poloxamer 407) and combinations thereof. Pluronic™polymers are commercially purchasable from BASF, USA and Germany.

In certain embodiments, anionic surfactants used in compositions and/or formulations described herein include, by way of non-limiting example, sodium laurylsulphate, sodium dodecyl sulfate (SDS), ammonium lauryl sulfate, alkyl sulfate salts, alkyl benzene sulfonate, and combinations thereof.

In some embodiments, the cationic surfactants used in compositions and/or formulations described herein include, by way of non-limiting example, benzalkonium chloride, benzethonium chloride, cetyl trimethylammonium bromide, hexadecyl trimethyl ammonium bromide, other alkyltrimethylammonium salts, cetylpyridinium chloride, polyethoxylated tallow and combinations thereof.

In certain embodiments, the thickeners used i in compositions and/or formulations described herein include, by way of non-limiting example, natural polysaccharides, semi-synthetic polymers, synthetic polymers, and combinations thereof. Natural polysaccharides include, by way of non-limiting example, acacia, agar, alginates, carrageenan, guar, arabic, tragacanth gum, pectins, dextran, gellan and xanthan gums. Semi-synthetic polymers include, by way of non-limiting example, cellulose esters, modified starches, modified celluloses, carboxymethylcellulose, methyl cellulose, ethyl cellulose, hydroxyethyl cellulose, hydroxypropyl cellulose and hydroxypropyl methylcellulose. Synthetic polymers include, by way of non-limiting example, polyoxyalkylenes, polyvinyl alcohol, polyacrylamide, polyacrylates, carboxypolymethylene (carbomer), polyvinylpyrrolidone (povidones), polyvinylacetate, polyethylene glycols and poloxamer. Other thickeners include, by way of nonlimiting example, polyoxyethyleneglycol isostearate, cetyl alcohol, Polyglycol 300 isostearate, propyleneglycol, collagen, gelatin, and fatty acids (e.g., lauric acid, myristic acid, palmitic acid, stearic acid, palmitoleic acid, linoleic acid, linolenic acid, oleic acid and the like).

In some embodiments, chelating agents used in the compositions and/or formulations described herein include, by way of non-limiting example, ethylenediaminetetraacetic acid (EDTA) or salts thereof, phosphates and combinations thereof.

In some embodiments, the concentration of the chelating agent or agents used in the rectal formulations described herein is a suitable concentration, e.g., about 0.1%, 0.15%, 0.2%, 0.25%, 0.3%, 0.4%, or 0.5% (w/v).

In some embodiments, preservatives used in compositions and/or formulations described herein include, by way of non-limiting example, parabens, ascorbyl palmitate, benzoic acid, butylated hydroxyanisole, butylated hydroxytoluene, chlorobutanol, ethylenediamine, ethylparaben, methylparaben, butyl paraben, propylparaben, monothioglycerol, phenol, phenylethyl alcohol, propylparaben, sodium benzoate, sodium propionate, sodium formaldehyde sulfoxylate, sodium metabisulfite, sorbic acid, sulfur dioxide, maleic acid, propyl gallate, benzalkonium chloride, benzethonium chloride, benzyl alcohol, chlorhexidine acetate, chlorhexidine gluconate, sorbic acid, potassium sorbitol, chlorbutanol, phenoxyethanol, cetylpyridinium chloride, phenylmercuric nitrate, thimerosol, and combinations thereof.

In certain embodiments, antioxidants used in compositions and/or formulations described herein include, by way of non-limiting example, ascorbic acid, ascorbyl palmitate, butylated hydroxyanisole, butylated hydroxytoluene, hypophosphorous acid, monothioglycerol, propyl gallate, sodium ascorbate, sodium sulfite, sodium bisulfite, sodium formaldehyde sulfoxylate, potassium metabisulphite, sodium metabisulfite, oxygen, quinones, t-butyl hydroquinone, erythorbic acid, olive (olea eurpaea) oil, pentasodium penetetate, pentetic acid, tocopheryl, tocopheryl acetate and combinations thereof.

In some embodiments, concentration of the antioxidant or antioxidants used in the rectal formulations described herein is sufficient to achieve a desired result, e.g., about 0.1%, 0.15%, 0.2%, 0.25%, 0.3%, 0.4%, or 0.5% (w/v).

The lubricating agents used in compositions and/or formulations described herein include, by way of non-limiting example, natural or synthetic fat or oil (e.g., a tris-fatty acid glycerate and the like). In some embodiments, lubricating agents include, by way of non-limiting example, glycerin (also called glycerine, glycerol, 1,2,3-propanetriol, and trihydroxypropane), polyethylene glycols (PEGs), polypropylene glycol, polyisobutene, polyethylene oxide, behenic acid, behenyl alcohol, sorbitol, mannitol, lactose, polydimethylsiloxane and combinations thereof.

In certain embodiments, mucoadhesive and/or bioadhesive polymers are used in the compositions and/or formulations described herein as agents for inhibiting absorption of the enteroendocrine peptide secretion enhancing agent across the rectal or colonic mucosa. Bioadhesive or mucoadhesive polymers include, by way of non-limiting example, hydroxypropyl cellulose, polyethylene oxide homopolymers, polyvinyl ether-maleic acid copolymers, methyl cellulose, ethyl cellulose, propyl cellulose, hydroxyethyl cellulose, hydroxypropyl cellulose, hydroxypropylmethyl cellulose, carboxymethylcellulose, polycarbophil, polyvinylpyrrolidone, carbopol, polyurethanes, polyethylene oxide-polypropyline oxide copolymers, sodium carboxymethyl cellulose, polyethylene, polypropylene, lectins, xanthan gum, alginates, sodium alginate, polyacrylic acid, chitosan, hyaluronic acid and ester derivatives thereof, vinyl acetate homopolymer, calcium polycarbophil, gelatin, natural gums, karaya, tragacanth, algin, chitosan, starches, pectins, and combinations thereof.

In some embodiments, buffers/pH adjusting agents used in compositions and/or formulations described herein include, by way of non-limiting example, phosphoric acid, monobasic sodium or potassium phosphate, triethanolamine (TRIS), BICINE, HEPES, Trizma, glycine, histidine, arginine, lysine, asparagine, aspartic acid, glutamine, glutamic acid, carbonate, bicarbonate, potassium metaphosphate, potassium phosphate, monobasic sodium acetate, acetic acid, acetate, citric acid, sodium citrate anhydrous, sodium citrate dihydrate and combinations thereof. In certain embodiments, an acid or a base is added to adjust the pH. Suitable acids or bases include, by way of non-limiting example, HCL, NaOH and KOH.

In certain embodiments, concentration of the buffering agent or agents used in the rectal formulations described herein is sufficient to achieve or maintain a physiologically desirable pH, e.g., about 0.1%, 0.2%, 0.3%, 0.4%, 0.5%, 0.6%, 0.8%, 0.9%, or 1.0% (w/w).

The tonicity modifiers used in compositions and/or formulations described herein include, by way of non-limiting example o, sodium chloride, potassium chloride, sodium phosphate, mannitol, sorbitol or glucose.

### Devices

In certain aspects of the methods and pharmaceutical compositions described herein, a device is used for rectal administration of the compositions and/or formulations described herein (e.g., the rectal gels, rectal foams, ememas and suppositories described herein). In certain embodiments, rectal gels or rectal enemas are administered using a bag or a syringe, while rectal foams are administered using a pressurized container.

In certain embodiments, a perfusion system is used to rectally administer the pharmaceutical compositions and/or formulations described herein. In some embodiments, the system comprises a tube surrounded by a semi-permeable membrane is rectally inserted and a solution containing a composition described herein is pumped into the membrane. In certain embodiments, the membrane expands to contact the rectal and/or colon walls, wherein the enterendocrine peptide secretion enhancing agents perfuse from the inside of the membrane to the outside. In certain embodiments, the solution is re-circulated as a continuous perfusion system.

### Oral Administration for Colonic Delivery

In certain aspects, the composition or formulation containing one or more enteroendocrine peptide secretion enhancing agents is orally administered for local delivery of an ASBTI, and/or an enteroendocrine peptide secretion enhancing agent, and/or an FXR agonist to the colon and/or rectum. Unit dosage forms of such compositions include a pill, tablet or capsules formulated for enteric delivery to colon. In certain embodiments, such pills, tablets or capsule contain the compositions described herein entrapped or embedded in microspheres. In some embodiments, microspheres include, by way of non-limiting example, chitosan microcores HPMC capsules and cellulose acetate butyrate (CAB) microspheres. In certain embodiments, oral dosage forms are prepared using conventional methods known to those in the field of pharmaceutical formulation. For example, in certain embodiments, tablets are manufactured using standard tablet processing procedures and equipment. An exemplary method for forming tablets is by direct compression of a powdered, crystalline or granular composition containing the active agent(s), alone or in combination with one or more carriers, additives, or the like. In alternative embodiments, tablets are prepared using wet-granulation or dry-granulation processes. In some embodiments, tablets are molded rather than compressed, starting with a moist or otherwise tractable material.

In certain embodiments, tablets prepared for oral administration contain various excipients, including, by way of non-limiting example, binders, diluents, lubricants, disintegrants, fillers, stabilizers, surfactants, preservatives, coloring agents, flavoring agents and the like. In some embodiments, binders are used to impart cohesive qualities to a tablet, ensuring that the tablet remains intact after compression. Suitable binder materials include, by way of non-limiting example, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), Veegum, and combinations thereof. In certain embodiments, diluents are utilized to increase the bulk of the tablet so that a practical size tablet is provided. Suitable diluents include, by way of non-limiting example, dicalcium phosphate, calcium sulfate, lactose, cellulose, kaolin, mannitol, sodium chloride, dry starch, powdered sugar and combinations thereof. In certain embodiments, lubricants are used to facilitate tablet manufacture; examples of suitable lubricants include, by way of non-limiting example, vegetable oils such as peanut oil, cottonseed oil, sesame oil, olive oil, corn oil, and oil of theobroma, glycerin, magnesium stearate, calcium stearate, stearic acid and combinations thereof. In some embodiments, disintegrants are used to facilitate disintegration of the tablet, and include, by way of non-limiting example, starches, clays, celluloses, algins, gums, crosslinked polymers and combinations thereof. Fillers include, by way of non-limiting example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. In certain embodiments, stabilizers are used to inhibit or retard drug decomposition reactions that include, by way of example, oxidative reactions. In certain embodiments, surfactants are anionic, cationic, amphoteric or nonionic surface active agents.

In some embodiments, ASBTIs, enteroendocrine peptide secretion enhancing agents, and/or FXR agonists described herein are orally administered in association with a carrier suitable for delivery of the enteroendocrine peptide secretion enhancing agents to the distal gastrointestinal tract (e.g., distal ileum, colon, and/or rectum).

In certain embodiments, a composition described herein comprises an ASBTI, an enteroendocrine peptide secretion enhancing agent, or an FXR agonist in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of abut 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent (e.g., bile acid) to the distal ileum. In some embodiments, a dosage form comprising an enteroendocrine peptide secretion enhancing agent (e.g., bile acid) is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pharmaceutical composition described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of Formula I. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

In further embodiments, a tablet or capsule comprising an ASBTI and/or an enteroendocrine peptide enhancing agent and/or an FXR agonist is film-coated for delivery to targeted sites within the gastrointestinal tract. Examples of enteric film coats include and are not limited to hydroxypropylmethylcellulose, polyvinyl pyrrolidone, hydroxypropyl cellulose, polyethylene glycol 3350, 4500, 8000, methyl cellulose, pseudo ethylcellulose, amylopectin and the like.

### Bile Acid Sequestrant

In certain embodiments, an oral formulation for use in any method described herein is, e.g., an ASBTI or an enteroendocrine peptide secretion enhancing agent or an FXR agonist in association with a labile bile acid sequestrant. A labile bile acid sequestrant is a bile acid sequestrant with a labile affinity for bile acids. In certain embodiments, a bile acid sequestrant described herein is an agent that sequesters (e.g., absorbs or is charged with) bile acid, and/or the salts thereof.

In specific embodiments, the labile bile acid sequestrant is an agent that sequesters (e.g., absorbs or is charged with) bile acid, and/or the salts thereof, and releases at least a portion of the absorbed or charged bile acid, and/or salts thereof in the distal gastrointestinal tract (e.g., the colon, ascending colon, sigmoid colon, distal colon, rectum, or any combination thereof). In certain embodiments, the labile bile acid sequestrant is an enzyme dependent bile acid sequestrant. In specific embodiments, the enzyme is a bacterial enzyme. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like. In some embodiments, the labile bile acid sequestrant is a time dependent bile acid sequestrant (i.e., the bile acid sequesters the bile acid and/or salts thereof and after a time releases at least a portion of the bile acid and/or salts thereof). In some embodiments, a time dependent bile acid sequestrant is an agent that degrades in an aqueous environment over time. In certain embodiments, a labile bile acid sequestrant described herein is a bile acid sequestrant that has a low affinity for bile acid and/or salts thereof, thereby allowing the bile acid sequestrant to continue to sequester bile acid and/or salts thereof in an environ where the bile acids and/or salts thereof are present in high concentration and release them in an environ wherein bile acids and/or salts thereof are present in a lower relative concentration. In some embodiments, the labile bile acid sequestrant has a high affinity for a primary bile acid and a low affinity for a secondary bile acid, allowing the bile acid sequestrant to sequester a primary bile acid or salt thereof and subsequently release a secondary bile acid or salt thereof as the primary bile acid or salt thereof is converted (e.g., metabolized) to the secondary bile acid or salt thereof. In some embodiments, the labile bile acid sequestrant is a pH dependent bile acid sequestrant. In some embodiments, the pH dependent bile acid sequestrant has a high affinity for bile acid at a pH of 6 or below and a low affinity for bile acid at a pH above 6. In certain embodiments, the pH dependent bile acid sequestrant degrades at a pH above 6.

In some embodiments, a bile acid sequestrant provided herein is cholestyramine, a hydrophilic polyacrylic quaternary ammonium anion exchange resin, which is known to be effective in reducing blood cholesterol levels. Cholestyramine, and various compositions including cholestyramine, are described, for example, in British Pat Nos. 929,391 and 1,286,949; and U.S. Pat. Nos. 3,383,281 ; 3,308,020; 3,769,399; 3,846,541 ; 3,974,272; 4,172,120; 4,252,790; 4,340,585; 4,814,354; 4,874,744; 4,895,723; 5,695,749; and 6,066,336, each of which are incorporated by reference herein. Cholestyramine is commercially available from Novopharm, USA Inc (Questrans Light), Upsher-Smith (PREVALITE (D) and Apothecon. As used herein, “cholestyramine” includes any such composition comprising cholestyramine, or pharmaceutically acceptable salts thereof. Questrans™Light (cholestyramine) is a non-absorbable anion binding resin FDA approved for the treatment of hypercholesterolemia. An amine polymer having a first substituent, bound to a first amine of the amine polymer, that includes a hydrophobic aliphatic moiety, and a second substituent, bound to a second amine of the amine polymer, that includes an aliphatic quaternary amine-containing moiety as described in U.S. Pat. Nos. 5,693,675 and 5,607,669, each of which are incorporated by reference herein. The salt of an alkylated and cross linked polymer comprising the reaction product of: (a) one or more cross linked polymers, or salts and copolymers thereof having a repeat unit selected from the group consisting of: (NR—CH2CH2)n (2) and (NR—CH2CH2-NR—CH2CH2-NR—CH2CHOH—CH2)n (3) where n is a positive integer and each R, independently, is H or a C1-C8alkyl group; (b) at least one aliphatic alkylating agent, said reaction product characterized in that: (i) at least some of the nitrogen atoms in said repeat units unreacted with said alkylating agent; (ii) less than 10 mol percent of the nitrogen atoms in said repeat units reacting with said alkylating agent forming quaternary ammonium units; and(iii) a fixed positive charge and one or more counter ions, such as Colesevelam and colesevelam hydrochloride.

In some embodiments, Suitable bile acid binders for such a combination therapy are resins, such as cholestyramine and cholestipol. One advantage is that the dose of bile acid binder might be kept lower than the therapeutic dose for treatment of cholesterolemia in single treatment comprising solely a bile acid binder. By a low dose of bile acid binder any possible side effects caused by poor tolerance of the patient to the therapeutic dose could also be avoided.

Another useful bile acid binder is a water insoluble non-toxic polymeric amine having a molecular weight in excess of 3,000, having the property of binding at least 30% of the available glycocholic acid within minutes when exposed to an aqueous solution of an equal weight of said acid, having a polymer skeleton inert to digestive enzymes, and having a water content greater than 65% after equilibration with air at 100% relative humidity, egg, cholestipol described in U.S. Pat. No. 3,383,281, which is incorporated by reference herein.

In some embodiments, a suitable bile acid binder is one of cholestyramine, cholestipol or colesevelam. In a preferred embodiment, provided herein is the use of colesevelam as the bile acid binder.

In some embodiments, labile bile acid sequestrants described herein include any compound, e.g., a macro-structured compound that can sequester bile acids and/or salts thereof through any suitable mechanism. For example, in certain embodiments, bile acid sequestrants sequester bile acids and/or salts thereof through ionic interactions, polar interactions, static interactions, hydrophobic interactions, lipophilic interactions, hydrophilic interactions, steric interactions, or the like. In certain embodiments, macrostructured compounds sequester bile acids and/or sequestrants by trapping the bile acids and/or salts thereof in pockets of the macrostructured compounds and, optionally, other interactions, such as those described above. In some embodiments, bile acid sequestrants (e.g., labile bile acid sequestrants) include, by way of non-limiting example, lignin, modified lignin, polymers, polycationic polymers and copolymers, polymers and/or copolymers comprising anyone one or more of N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof, or any combination thereof.

Covalent Linkage of the Drug with a Carrier

In some embodiments, strategies used for colon targeted delivery include, by way of non-limiting example, covalent linkage of the ASBTI and/or the enteroendocrine peptide secretion enhancing agents to a carrier, coating the dosage form with a pH-sensitive polymer for delivery upon reaching the pH environment of the colon, using redox sensitive polymers, using a time released formulation, utilizing coatings that are specifically degraded by colonic bacteria, using bioadhesive system and using osmotically controlled drug delivery systems.

In certain embodiments of such oral administration of a composition containing an ASBTI and/or an enteroendocrine peptide secretion enhancing agent and/or an FXR agonist described herein involves covalent linking to a carrier wherein upon oral administration the linked moiety remains intact in the stomach and small intestine. Upon entering the colon the covalent linkage is broken by the change in pH, enzymes, and/or degradation by intestinal microflora. In certain embodiments, the covalent linkage between the ASBTI and/or enteroendocrine peptide secretion enhancing agent and the carrier includes, by way of non-limiting example, azo linkage, glycoside conjugates, glucuronide conjugates, cyclodextrin conjugates, dextran conjugates, and amino-acid conjugates (high hydrophilicity and long chain length of the carrier amino acid).

Coating with Polymers: pH-Sensitive Polymers

In some embodiments, the oral dosage forms described herein are coated with an enteric coating to facilitate the delivery of an ASBTI and/or an enteroendocrine peptide secretion enhancing agent to the colon and/or rectum. In certain embodiments, an enteric coating is one that remains intact in the low pH environment of the stomach, but readily dissolved when the optimum dissolution pH of the particular coating is reached which depends upon the chemical composition of the enteric coating. The thickness of the coating will depend upon the solubility characteristics of the coating material. In certain embodiments, the coating thicknesses used in such formulations described herein range from about 25 μm to about 200 μm.

In certain embodiments, the compositions or formulations described herein are coated such that an enteroendocrine peptide secretion enhancing agent of the composition or formulation is delivered to the colon and/or rectum without absorbing at the upper part of the intestine. In a specific embodiment, specific delivery to the colon and/or rectum is achieved by coating of the dosage form with polymers that degrade only in the pH environment of the colon. In alternative embodiments, the composition is coated with an enteric coat that dissolves in the pH of the intestines and an outer layer matrix that slowly erodes in the intestine. In some of such embodiments, the matrix slowly erodes until only a core composition comprising an enteroendocrine peptide secretion enhancing agent (and, in some embodiments, an absorption inhibitor of the agent) is left and the core is delivered to the colon and/or rectum.

In certain embodiments, pH-dependent systems exploit the progressively increasing pH along the human gastrointestinal tract (GIT) from the stomach (pH 1-2 which increases to 4 during digestion), small intestine (pH 6-7) at the site of digestion and it to 7-8 in the distal ileum. In certain embodiments, dosage forms for oral administration of the compositions described herein are coated with pH-sensitive polymer(s) to provide delayed release and protect the enteroendocrine peptide secretion enhancing agents from gastric fluid. In certain embodiments, such polymers are be able to withstand the lower pH values of the stomach and of the proximal part of the small intestine, but disintegrate at the neutral or slightly alkaline pH of the terminal ileum and/or ileocecal junction. Thus, in certain embodiments, provided herein is an oral dosage form comprising a coating, the coating comprising a pH-sensitive polymer. In some embodiments, the polymers used for colon and/or rectum targeting include, by way of non-limiting example, methacrylic acid copolymers, methacrylic acid and methyl methacrylate copolymers, Eudragit L100, Eudragit S100, Eudragit L-30D, Eudragit FS-30D, Eudragit L100-55, polyvinylacetate phthalate, hyrdoxypropyl ethyl cellulose phthalate, hyrdoxypropyl methyl cellulose phthalate 50, hyrdoxypropyl methyl cellulose phthalate 55, cellulose acetate trimelliate, cellulose acetate phthalate and combinations thereof.

In certain embodiments, oral dosage forms suitable for delivery to the colon and/or rectum comprise a coating that has a biodegradable and/or bacteria degradable polymer or polymers that are degraded by the microflora (bacteria) in the colon. In such biodegradable systems suitable polymers include, by way of non-limiting example, azo polymers, linear-type-segmented polyurethanes containing azo groups, polygalactomannans, pectin, glutaraldehyde crosslinked dextran, polysaccharides, amylose, guar gum, pectin, chitosan, inulin, cyclodextrins, chondroitin sulphate, dextrans, locust bean gum, chondroitin sulphate, chitosan, poly (-caprolactone), polylactic acid and poly(lactic-co-glycolic acid).

In certain embodiments of such oral administration of compositions containing one or more ASBTIs and/or enteroendocrine peptide secretion enhancing agents described herein, the compositions are delivered to the colon without absorbing at the upper part of the intestine by coating of the dosage forms with redox sensitive polymers that are degraded by the microflora (bacteria) in the colon. In such biodegradable systems such polymers include, by way of non-limiting example, redox-sensitive polymers containing an azo and/or a disulfide linkage in the backbone.

In some embodiments, compositions formulated for delivery to the colon and/or rectum are formulated for time-release. In some embodiments, time release formulations resist the acidic environment of the stomach, thereby delaying the release of the enteroendocrine peptide secretion enhancing agents until the dosage form enters the colon and/or rectum.

In certain embodiments the time released formulations described herein comprise a capsule (comprising an enteroendocrine peptide secretion enhancing agent and an optional absorption inhibitor) with hydrogel plug. In certain embodiments, the capsule and hydrogel plug are covered by a water-soluble cap and the whole unit is coated with an enteric polymer. When the capsule enters the small intestine the enteric coating dissolves and the hydrogels plug swells and dislodges from the capsule after a period of time and the composition is released from the capsule. The amount of hydrogel is used to adjust the period of time to the release the contents.

In some embodiments, provided herein is an oral dosage form comprising a multi-layered coat, wherein the coat comprises different layers of polymers having different pH-sensitivities. As the coated dosage form moves along GIT the different layers dissolve depending on the pH encountered. Polymers used in such formulations include, by way of non-limiting example, polymethacrylates with appropriate pH dissolution characteristics, Eudragit® RL and Eudragit®RS (inner layer), and Eudragit® FS (outer layer). In other embodiments the dosage form is an enteric coated tablets having an outer shell of hydroxypropylcellulose or hydroxypropylmethylcellulose acetate succinate (HPMCAS).

In some embodiments, provided herein is an oral dosage form that comprises coat with cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and combinations thereof.

### Combination Therapy

In certain instances, provided herein are combination compositions and/or therapies comprising any compound described herein and an additional therapeutic agent. In some embodiments, the additional therapeutic agent is a L-cell endocrine peptide enhancer. In some instances, the L-cell endocrine peptide enhancer is a GLP-1 enhancer. In some embodiments, the GLP-1 enhancer is GLP-1, a GLP-1 secretion enhancer, a GLP-1 degradation inhibitor, the like, or a combination thereof. In certain instances, enhanced GLP-1 concentration provides regeneration of intestinal lining and/or heals injury to the gastrointestinal structures and/or reduces induction of cytokines and/or enhances the adaptive process, attenuates intestinal injury, reduces bacterial translocation, inhibits the release of free radical oxygen, or any combination thereof. In some instances, the L-cell endocrine peptide enhancer is a PYY enhancer. In some instances, the L-cell endocrine peptide enhancer is an oxyntomodulin enhancer. In some instances, enhanced PYY or oxyntomodulin secretion heals injury to pancreas.

### TGR5 Receptor Modulators

In some instances, the additional therapeutic agent modulates bile acid receptors in the gastrointestinal lumen. In some embodiments, the additional therapeutic agent agonizes or partially agonizes bile acid receptors (e.g., TGR5 receptors or Farnesoid-X receptors) in the gastrointestinal tract. In some embodiments, the additional therapeutic agent is a bile acid analog. In certain instances the additional therapeutic agent is a TGR5 agonist. In certain instances, administration of a TGR5 agonist in combination with any of the compounds described herein enhances the secretion of enteroendocrine peptides from L-cells. TGR5 modulators (e.g., agonists) include, and are not limited to, the compounds described in, WO 2008/091540, WO 2008/067219 and U.S. Appl. No. 2008/0221161. Biguanides

In some embodiments, the additional therapeutic agent is a biguanide. In some instances, biguanides reduce bile acid reuptake in the GI tract. Examples of biguanides include and are not limited to metformin, buformin, phenformin, proguanil or the like.

### Enteroendocrine Peptides

In some embodiments, the additional therapeutic agent is an enteroendocrine peptide. In some embodiments, enteroendocrine peptides heals injury to pancreas. Examples of enteroendocrine peptides that are administered as additional therapeutic agents include and are not limited to GLP-1 or GLP-1 analogs such as Taspoglutide® (Ipsen), or the like.

### Pain Medication

In some embodiments, the additional therapeutic agent is an agent that treats pain. Examples of pain therapeutics include and are not limited to analgesics (e.g., acetaminophen); non-steroidal anti-inflammatory drugs (e.g., ibuprofen, naproxen, anti-inflammatory steroids (e.g., dexamethasone, prednisolone and the like), celecoxib, rofecoxib and the like; or narcotics or opiates (e.g., codeine, hydrocodone, morphine, fentanyl, methadone, oxycodone and the like).

### Pancreatic Enzymes

In some embodiments, the additional therapeutic agent is a pancreatic enzyme. Pancreatic juice, composed of the secretions of both ductal and acinar cells, is composed of several enzymes and/or hormones such as, for example, trypsinogen (which is an inactive(zymogenic) protease that, once activated in the duodenum, into trypsin, breaks down proteins at the basic amino acids); chymotrypsinogen (which is an inactive(zymogenic) protease that once activated by duodenal enterokinase, breaks down proteins at their aromatic amino acids); carboxypeptidase (which is a protease that takes off the terminal amino acid group from a protein); pancreatic lipase that degrades triglycerides into fatty acids and glycerol; pancreatic amylase that, degrades most carbohydrates; amylin, and the like. Examples of certain pancreatic enzymes that are available as oral supplements include and are not limited to Creon® (pancrealipase) capsules, Pancreaze™ (pancrealipase enteric coated) capsules, and Zenpep™(pancrealipase delayed release) capsules.

Combination Therapy with ASBTI and DPP-IV Inhibitor

In specific embodiments, the additional therapeutic agent inhibits degradation of L-cell enteroendocrine peptides. In certain embodiments, the additional therapeutic agent is a DPP-IV inhibitor. In certain instances, administration of an ASBTI to an individual in need thereof enhances the secretion of GLP-1; administration of a DPP-IV inhibitor in combination with the ASBTI reduces or inhibits degradation of GLP-1 thereby prolonging the therapeutic benefit of enhanced levels of GLP-1.

DPP-IV inhibitors suitable for use with the methods described herein include and are not limited to (2S)-1-{2-[(3-hydroxy-1-adamantyl)amino]acetyl}pyrrolidine-2-carbonitrile (vildagliptin), (3R)-3-amino-1-[9-(trifluoromethyl)-1,4,7,8-tetrazabicyclo[4.3.0]nona-6,8-dien-4-yl]-4-(2,4,5-trifluorophenyl)butan-1-one (sitagliptin), (1S,3S,5S)-2-[(2S)-2-amino-2-(3-hydroxy-1-adamantyl)acetyl]-2-azabicyclo[3.1.0]hexane-3-carbonitrile (saxagliptin), and 2-({6-[(3R)-3-aminopiperidin-1-yl]-3-methyl-2,4-dioxo-3,4-dihydropyrimidin-1(2H)-yl}methyl)benzonitrile (alogliptin).

In certain embodiments, an ASBTI and a second active ingredient are used such that the combination is present in a therapeutically effective amount. That therapeutically effective amount arises from the use of a combination of an ASBTI and the other active ingredient (e.g., a DPP-IV inhibitor) wherein each is used in a therapeutically effective amount, or by virtue of additive or synergistic effects arising from the combined use, each can also be used in a subclinical therapeutically effective amount, i.e., an amount that, if used alone, provides for reduced effectiveness for the therapeutic purposes noted herein, provided that the combined use is therapeutically effective. In some embodiments, the use of a combination of an ASBTI and any other active ingredient as described herein encompasses combinations where the ASBTI or the other active ingredient is present in a therapeutically effective amount, and the other is present in a subclinical therapeutically effective amount, provided that the combined use is therapeutically effective owing to their additive or synergistic effects. As used herein, the term “additive effect” describes the combined effect of two (or more) pharmaceutically active agents that is equal to the sum of the effect of each agent given alone. A syngergistic effect is one in which the combined effect of two (or more) pharmaceutically active agents is greater than the sum of the effect of each agent given alone. Any suitable combination of an ASBIT with one or more of the aforementioned other active ingredients and optionally with one or more other pharmacologically active substances is contemplated as being within the scope of the methods described herein.

In some embodiments, the particular choice of compounds depends upon the diagnosis of the attending physicians and their judgment of the condition of the individual and the appropriate treatment protocol. The compounds are optionally administered concurrently (e.g., simultaneously, essentially simultaneously or within the same treatment protocol) or sequentially, depending upon the nature of the disease, disorder, or condition, the condition of the individual, and the actual choice of compounds used. In certain instances, the determination of the order of administration, and the number of repetitions of administration of each therapeutic agent during a treatment protocol, is based on an evaluation of the disease being treated and the condition of the individual.

In some embodiments, therapeutically-effective dosages vary when the drugs are used in treatment combinations. Methods for experimentally determining therapeutically-effective dosages of drugs and other agents for use in combination treatment regimens are described in the literature.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g, from more than zero weeks to less than four weeks. In addition, the combination methods, compositions and formulations are not to be limited to the use of only two agents; the use of multiple therapeutic combinations are also envisioned (including two or more compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy described herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In certain embodiments, provided herein are combination therapies. In certain embodiments, the compositions described herein comprise an additional therapeutic agent. In some embodiments, the methods described herein comprise administration of a second dosage form comprising an additional therapeutic agent. In certain embodiments, combination therapies the compositions described herein are administered as part of a regimen. Therefore, additional therapeutic agents and/or additional pharmaceutical dosage form can be applied to a patient either directly or indirectly, and concomitantly or sequentially, with the compositions and formulations described herein.

### Kits

In another aspect, provided herein are kits containing a device for rectal administration pre-filled a pharmaceutical composition described herein. In certain embodiments, kits contain a device for rectal administration and a pharmaceutical composition (e.g., a rectal dosage form) as described herein. In certain embodiments the kits includes prefilled bags for administration of rectal enemas, while in other embodiments the kits include prefilled bags for administration of rectal gels. In certain embodiments the kits includes prefilled syringes for administration of rectal enemas, while in other embodiments the kits include prefilled syringes for administration of rectal gels. In certain embodiments the kits includes prefilled pressurized cans for administration of rectal foams.

### Pharmaceutical Compositions

Provided herein, in certain embodiments, is a pharmaceutical composition comprising a therapeutically effective amount of any compound described herein. In certain instances, the pharmaceutical composition comprises an ASBT inhibitor (e.g., any ASBTI described herein).

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a compound described herein, such as, for example, a compound of Formula I-VI, with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the compounds described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

In certain embodiments, a pharmaceutical compositions described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” includes, in some embodiments, a pharmaceutically acceptable excipient and is selected on the basis of compatibility with compounds described herein, such as, compounds of any of Formula I-VII, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in certain embodiments, the pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Release in Distal Ileum and/or Colon

In certain embodiments, a dosage form comprises a matrix (e.g., a matrix comprising hypermellose) that allows for controlled release of an active agent in the distal jejunum, proximal ileum, distal ileum and/or the colon. In some embodiments, a dosage form comprises a polymer that is pH sensitive (e.g., a MMX™ matrix from Cosmo Pharmaceuticals) and allows for controlled release of an active agent in the ileum and/or the colon. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of about 7 to about 8). In some embodiments, a dosage form suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an ASBTI to the distal ileum. In some embodiments, a dosage form comprising an ASBTI is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the ileum and/or the colon. In some embodiments, bacterially activated systems are suitable for targeted delivery to the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of Formula I-VI. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

An ASBT inhibitor (e.g., a compound of Formula I-VI) is used in the preparation of medicaments for the prophylactic and/or therapeutic treatment of pancreatitis. A method for treating any of the diseases or conditions described herein in an individual in need of such treatment, involves administration of pharmaceutical compositions containing at least one ASBT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

### Screening Process

Provided in certain embodiments herein are processes and kits for identifying compounds suitable for treating pancreatitis mediated by L-cell enteroendocrine peptides. In certain embodiments, provided herein are assays for identifying compounds that selectively inhibit the ASBT, or enhance the secretion of L-cell enteroendocrine peptides, or FXR agonists, or a combination thereof by:
a. providing cells that are a model of intestinal L-cells (e.g., SLC-1 cells, GLUTag cells, NCI-H719 cells);
b. contacting the cells with a compound (e.g., a compound as described herein);
c. detecting or measuring the effect of the compound on the secretion of enteroendocrine peptides (e.g., GLP-1, GLP-2) from the cells.

In certain embodiments, provided herein are assays for identifying compounds that are non-systemic compounds by
a. providing cells that are a model of intestinal permeability (e.g., Caco-2 cells);
b. culturing the cells as a monolayer on semi-permeable plastic supports that are fitted into the wells of multi-well culture plates;
c. contacting the apical or basolateral surface of the cells with a compound (e.g., a compound as described herein) and incubating for a suitable length of time;
d. detecting or measuring the concentration of the compound on both sides of the monolayer by liquid-chromatography-mass spectrometry (LC-MS) and computing intestinal permeability of the compound.

In certain embodiments, non-systemic compounds are identified by suitable parallel artificial membrane permeability assays (PAMPA).

In certain embodiments, non-systemic compounds are identified by use of isolated vascular-perfused gut preparations.

In certain embodiments, provided herein are assays for identifying compounds that inhibit recycling of bile acid salts by
a. providing cells that are a model of intestinal cells with apical bile acid transporters (e.g., BHK cells, CHO cells);
b. incubating the cells with a compound (e.g., a compound as described herein) and/or a radiolabeled bile acid (e.g., ¹⁴C taurocholate) for a suitable length of time;
c. washing the cells with a suitable buffer (e.g. phosphate buffered saline);
d. detecting or measuring the residual concentration of the radiolabeled bile acid in the cells.

### EXAMPLES

### Example 1

### Synthesis of 1-phenethyl-1-((1,4-diazabicyclo[2.2.2]octanyl)pentyl)imidodicarbonimidic diamide, iodide salt

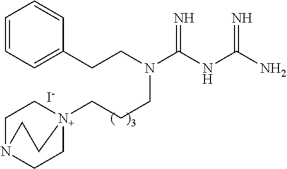

### Step 1: Synthesis of 5-(1,4-diazabicyclo[2.2.2]octanyl)-1-iodo pentane, iodide salt

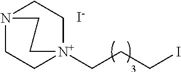

1,4-diazabicyclo[2.2.2]octane is suspended in THF. Diiodopentane is added dropwise and the mixture is refluxed overnight. The reaction mixture is filtered.

### Step 2: Synthesis of N-phenethyl-5-(1,4-diazabicyclo[2.2.2]octanyl)-1-iodo pentane, iodide salt

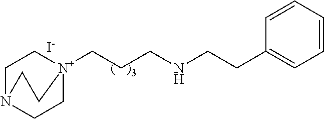

5-(1,4-diazabicyclo[2.2.2]octanyl)-1-iodo pentane, iodide salt is suspended in acetonitrile. Phenethylamine is added dropwise and the mixture is refluxed overnight. The reaction mixture is filtered.

### Step 3: Synthesis of 1-phenethyl-1-((1,4-diazabicyclo[2.2.2]octanyl)pentyl)imidodicarbonimidic diamide, iodide salt

N-phenethyl-5-(1,4-diazabicyclo[2.2.2]octanyl)-1-iodo pentane, iodide salt is heated with dicyanodiamide in n-butanol for 4 h. The reaction mixture is concentrated under reduced pressure.

The compounds in Table 1 are prepared using methods as described herein, and using appropriate starting materials.

TABLE 1

| Compound | |
|---|---|
| No. | Structure |
| 1 | 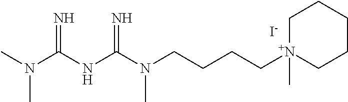 [SMILES: CN(C)C(=N)NC(=N)N(C)CCCC[N+]1(C)CCCCC1 ; [I-]] |
| 2 | 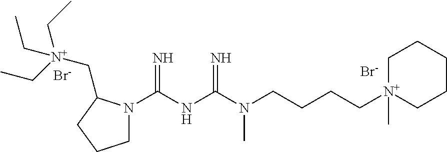 [SMILES: CC[N+](CC)(CC)CC1CCCN1C(=N)NC(=N)N(C)CCCC[N+]1(C)CCCCC1 ; [Br-] ; [Br-]] |
| 3 | 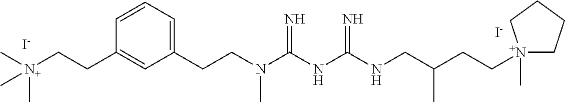 [SMILES: CC(CC[N+]1(C)CCCC1)CNC(=N)NC(=N)N(C)CCc1cccc(CC[N+](C)(C)C)c1 ; [I-] ; [I-]] |
| 4 | 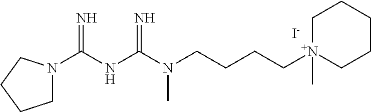 [SMILES: CN(CCCC[N+]1(C)CCCCC1)C(=N)NC(=N)N1CCCC1 ; [I-]] |
| 5 | 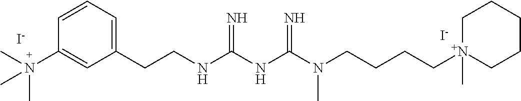 |
| 6 | 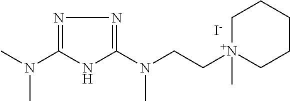 [SMILES: CN(C)c1nnc(N(C)CC[N+]2(C)CCCCC2)[nH]1 ; [I-]] |
| 7 | 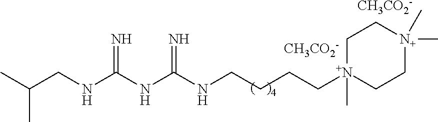 |
| 8 | 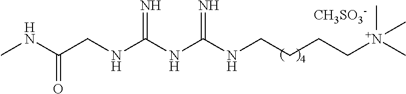 |
| 9 | 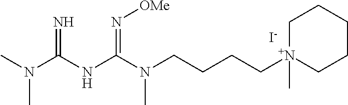 [SMILES: CO/N=C(/NC(=N)N(C)C)N(C)CCCC[N+]1(C)CCCCC1 ; [I-]] |
| 10 | 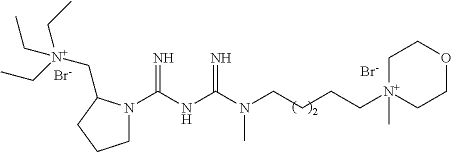 |
| 11 | 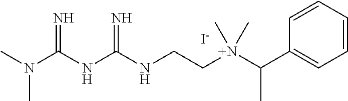 [SMILES: CC(c1ccccc1)[N+](C)(C)CCNC(=N)NC(=N)N(C)C ; [I-]] |

### Example 2

### In Vitro Assay for Inhibition of ASBT-Mediated Bile Acid Uptake

Baby hamster kidney (BHK) cells are transfected with cDNA of human ASBT. The cells are seeded in 96-well tissue culture plates at 60,000 cells/well. Assays are run within 24 hours of seeding.

On the day of the assay the cell monolayer is washed with 100 mL of assay buffer. The test compound is added to each well along with 6 mM [¹⁴C]taurocholate in assay buffer (final concentration of 3 mM [¹⁴C]taurocholate in each well). The cell cultures are incubated for 2 h at 37° C. The wells are washed with PBS. Scintillation counting fluid is added to each well, the cells are shaken for 30 minutes prior to measuring amount of radioactivity in each well. A test compound that has significant ASBT inhibitory activity provides an assay wherein low levels of radioactivity are observed in the cells.

### Example 3

### In Vitro Assay for Secretion of GLP-2

Human NCI-H716 cells are used as a model for L-cells. Two days before each assay experiment, cells are seeded in 12-well culture plates coated with Matrigel® to induce cell adhesion. On the day of the assay, cells are washed with buffer. The cells are incubated for 2 hours with medium alone, or with test compound. The extracellular medium is assayed for the presence of GLP-2. Peptides in the medium are collected by reverse phase adsorption and the extracts are stored until assay. The presence of GLP-2 is assayed using ELISA. The detection of increased levels of GLP-2 in a well containing a test compound identifies the test compound as a compound that can enhance GLP-2 secretions from L-cells.

### Example 4

### In Vivo Bioavailability Assay

The test compounds are solubilized in saline solutions. Sprague Dawley rats are dosed at 2-10 mg/kg body weight by iv and oral dosing. Peripheral blood samples are taken from the femoral artery at selected time periods up to 8 hours. Plasma concentrations of the compounds are determined by quantitative HPLC and/or mass spectrometry. Clearance and AUC values are determined for the compounds.

For oral dosing, bioavailabilty is calculated by also drawing plasma samples from the portal vein. Cannulae are inserted in the femoral artery and the hepatic portal vein to obtain estimates of total absoprtion of drug without first-pass clearance in the liver. The fraction absorbed (F) is calculated by

F=AUCₚₒ/AUCᵢᵥ

### Example 5

### Assay to Determine Ileal Intraenterocyte and Luminal Bile Acid Levels

Ileal luminal bile acid levels in SD rats are determined by flushing a 3-cm section of distal ileum with sterile, cold PBS. After flushing with additional PBS, the same section of ileum is weighed and then homogenized in fresh PBS for determination of interenterocyte bile acid levels. A LC/MS/MS system is used to evaluate cholic acid, DCA, LCA, chnodeoxycholic acid, and ursodeoxycholic acid levels.

### Example 6

### In Vivo Effects of ASBT Inhibitor SC-435 on Plasma Active GLP-1 Levels in Pancreatitis Treatment

Reduction of pancreatic enzymes secretion is crucial factor for treatment of pancreatitis. GLP-1 reduces exocrine pancreatic secretion and has been demonstrated to improve and ameliorate pancreatitis. As such, it was our goal to increase plasma GLP-1 levels to decrease the markers of pancreatitis.

Animals:

Male Sprague Dawley rats (HSD) 12 weeks old were fasted overnight for 16 hours.

Test Compound:

SC-435 (racemate form synthesized at Nanosyn Inc. Menlo Park, Calif.) in 1 ml of saline administrated orally via gavage tube (n=5 per group).

Dosage:

SC-435 doses of 0, 3, 30 or 100 mg/kg in 1 ml water.

Blood Collection:

Blood samples (200 μl) taken from the caudal vein with capillary tube at 0, 1, 3 and h after compound administration for testing of plasma active GLP-1 levels (ELISA, Millipore Inc.)

Plasma Collection:

Blood was collected in ice-cooled EDTA vial. Immediately (<30 seconds) after collection DPP-IV inhibitor added (10mkl per 1 ml blood). Samples were centrifuged immediately at 1000×g for 10 minutes in refrigerated centrifuge. Plasma was stored at −70° C. until evaluation.

Results:

SC-435 dose-dependently increased 5-hour integrated GLP-1 concentrations 2.5 fold vs. vehicle. Peak GLP-1 of 30-36 pM observed 3-5 hours after SC-435 administration (FIG. 1).

Conclusion:

Oral administration of the ASBTi's produced significant and dose-dependent increase of GLP-1 secretion, which is associated with treatment and prevention of pancreatitis. ASBTIs would be valuable in the treatment of pancreatitis.

### Example 7

### Animal Model to Determine Effect of Therapy on Pancreatitis

A modified protocol for a noninvasive model of severe acute pancreatitis in rats described in Surgery 2007, 142, pp 327-336 is used.

Wistar rats are infused intravenously with cerulein or a combination of cerulein and enterokinase. Saline (154-mmol/L NaCl) is infused in controls. Intrapancreatic protease activation and the release of cytokines is correlated with the severity of organ injury. Pancreatic injuries are determined at 6 h by measurement of IL-6 and amylase levels in serum and histology.

At 6 hours, the animals are orally administered a composition comprising compound 100A or compound 100B or a composition comprising bile cid mimic INT-777. Other compounds disclosed herein are tested.

At 24 hours histologic evaluation includes pancreatic hemorrhage, necrosis, and leukocyte infiltration in pancreas of animals. IL-6 and amylase levels in serum are correlated with the severity of pancreatitis. Lower levels of amylase and/or IL-6 are indicative of a therapeutic effect.

### Example 8

Investigation of orally delivered 1-[4-[4-[(4R,5R)-3,3-dibutyl-7-(dimethylamino)-2,3,4,5-tetrahydro-4-hydroxy-1,1-dioxido-1-benzothiepin-5-yl]phenoxy]butyl]-4-aza-1-azoniabicyclo[2.2.2]octane methane sulfonate (Compound 100B) and metformin in combination with DPP-IV inhibitor on plasma GLP-1 levels in normal rats

12-week-old male HSD rats are fasted for 16 h and given oral dose of 0, 3, 30, 100 mg/kg of the ASBTI 1-[4-[4-[(4R,5R)-3,3-dibutyl-7-(dimethylamino)-2,3,4,5-tetrahydro-4-hydroxy-1,1-dioxido-1-benzothiepin-5-yl]phenoxy]butyl]-4-aza-1-azoniabicyclo[2.2.2]octane methane sulfonate (Synthesized by Nanosyn Inc., CA, USA) or metformin (Control, 0, 3, 30, 100, 300 mg/kg) in saline and a dose of 30 mg/kg DPP-IV inhibitor sitaglipin in a mixture of valine-pyrrolidine in water (n=5 per group). Blood samples in volume of 0.6 ml for each time point are taken from the caudal vein with a heparinized capillary tube 0, 1, 3 and 5 h after the administration of compounds and plasma GLP-1 levels are determined. Aprotinin and 10 μl of DPP-IV inhibitor per ml of blood are used for blood sample preservation during 10 min centrifugation and for storage at −70° C. or below. GLP-1 (Active pM) is tested by Millipore ELISA Kits (Millipore Corporation, 290 Concord Road, Billerica, Mass.).

### Example 9

### Tablet Formulation

10 kg of a compound of Formula I-VII is first screened through a suitable screen (e.g. 500 micron). 25 kg Lactose monohydrate, 8 kg hydroxypropylmethyl cellulose, the screened compound of Formula I-VII and 5 kg calcium hydrogen phosphate (anhydrous) are then added to a suitable blender (e.g. a tumble mixer) and blended. The blend is screened through a suitable screen (e.g. 500 micron) and reblended. About 50% of the lubricant (2.5 kg, magnesium stearate) is screened, added to the blend and blended briefly. The remaining lubricant (2 kg, magnesium stearate) is screened, added to the blend and blended briefly. The granules are screened (e.g. 200 micron) to obtain granulation particles of the desired size. In some embodiments, the granules are optionally coated with a drug release controlling polymer such as polyvinylpyrrolidine, hydroxypropylcellulose, hydroxypropylmethyl cellulose, methyl cellulose, or a methacrylic acid copolymer, to provide an extended release formulation. The granules are filled in gelatin capsules.

### Example 10

### Rectal Foams

### a) 500 mM Sodium Taurocholate

### Preparation Method:

Using a stainless steel dissolving vessel fitted with a propeller stirrer and turboemulsifier 26.88 grams of sodium taurocholate, 0.25 grams of potassium metabisulphite, 0.3 grams EDTA (disodium salt), 0.38 grams of sodium benzoate and 0.2 grams of xanthan gum are dissolved in 100 mL of purified water. While stirring, 4 grams of Polysorbate 20 and 4 grams of Polyglycol 300 isostearate are added and stirring is continued for 15 minutes. The suspension is then pumped into an aerosol cans and is immediately sealed by clinching the dispenser valve. The can is then pressurized by pumping 6.5 grams of Freon 12 and 3.5 grams of Freon 114 into the can.

### b) 500 mM Sodium Glycocholate

### Preparation Method:

Using a stainless steel dissolving vessel fitted with a propeller stirrer and turboemulsifier 24.38 grams of sodium glycocholate, 0.25 grams of potassium metabisulphite, 0.3 grams EDTA (disodium salt), 0.38 grams of sodium benzoate and 0.2 grams of xanthan gum are dissolved in 100 mL of purified water. While stirring, 4 grams of Polysorbate 20 and 4 grams of Polyglycol 300 isostearate are added and stirring is continued for 15 minutes. The suspension is then pumped into an aerosol cans and is immediately sealed by clinching the dispenser valve. The can is then pressurized by pumping 6.5 grams of Freon 12 and 3.5 grams of Freon 114 into the can.

### c) No Bile Salt (Control)

### Preparation Method:

Using a stainless steel dissolving vessel fitted with a propeller stirrer and turboemulsifier 0.25 grams of potassium metabisulphite, 0.3 grams EDTA (disodium salt), 0.38 grams of sodium benzoate and 0.2 grams of xanthan gum are dissolved in 100 mL of purified water. While stirring, 4 grams of Polysorbate 20 and 4 grams of Polyglycol 300 isostearate are added and stirring is continued for 15 minutes. The suspension is then pumped into an aerosol cans and is immediately sealed by clinching the dispenser valve. The can is then pressurized by pumping 6.5 grams of Freon 12 and 3.5 grams of Freon 114 into the can.

### Example 11

### Rectal Enemas

### a) 500 mM Sodium Taurocholate

### Preparation Method:

Using a stainless steel dissolving vessel fitted with a propeller stirrer 26.88 grams of sodium taurocholate, 0.25 grams of potassium metabisulphite, 0.3 grams EDTA (disodium salt), 0.38 grams of sodium benzoate are dissolved in 100 mL of purified water and stirring is continued for 10 minutes. The solution is then pulled into a syringe.

### b) 500 mM Sodium Glycocholate

### Preparation Method:

Using a stainless steel dissolving vessel fitted with a propeller stirrer and turboemulsifier 24.38 grams of sodium glycocholate, 0.25 grams of potassium metabisulphite, 0.3 grams EDTA (disodium salt), 0.38 grams of sodium benzoate are dissolved in 100 mL of purified water and stirring is continued for 10 minutes. The solution is then pulled into a syringe.

c) No Bile salt (Control)

### Preparation Method:

Using a stainless steel dissolving vessel fitted with a propeller stirrer and turboemulsifier 0.25 grams of potassium metabisulphite, 0.3 grams EDTA (disodium salt), 0.38 grams of sodium benzoate are dissolved in 100 mL of purified water and stirring is continued for 10 minutes. The solution is then pulled into a syringe.

### Example 12

### Rectal Suppositories

### a) Sodium Taurocholate

### Preparation Method:

Using a stainless steel dissolving vessel fitted with a propeller stirrer 2.69 grams of sodium taurocholate and 0.1 grams of methyl cellulose are added to 10 grams of higher saturated fatty acid triglycerides (Witepsol™S55; Dynamic Novel Aktiengesellschaft, West Germany) and the combination is melted at 50 C and stirred. While the composition is a liquid it is filled into suppository containers for rats (50 mg per container) and then quenched in ice-water.

### b) 500 mM Sodium Glycocholate

### Preparation Method:

Using a stainless steel dissolving vessel fitted with a propeller stirrer 2.69 grams of sodium glycocholate and 0.1 grams of methyl cellulose are added to 10 grams of higher saturated fatty acid triglycerides (Witepsol™S55; Dynamic Novel Aktiengesellschaft, West Germany) and the combination is melted at 50 C and stirred. While the composition is a liquid it is filled into suppository containers for rats (50 mg per container) and then quenched in ice-water.

### c) No Bile Salt (Control)

### Preparation Method:

Using a stainless steel dissolving vessel fitted with a propeller stirrer 0.1 grams of methyl cellulose is added to 10 grams of higher saturated fatty acid triglycerides (Witepsol™S55; Dynamic Novel Aktiengesellschaft, West Germany) and the combination is melted at 50 C and stirred. While the composition is a liquid it is filled into suppository containers for rats (50 mg per container) and then quenched in ice-water.

### Example 13

### Rectal Gels—Sodium Taurocholate/Control

### a) 500 mM Sodium Taurocholate

### Preparation Method:

Using a stainless steel dissolving vessel fitted with a propeller stirrer 26.88 grams of sodium taurocholate and 1 gram of methyl cellulose are dissolved in 100 mL of purified water and stirred for 15 minutes. 6 syringes connected to gavage tubes were then each filled with 3 mL of the composition.

### b) No Bile Salt (Control)

### Preparation Method:

Using a stainless steel dissolving vessel fitted with a propeller stirrer 1 gram of methyl cellulose is dissolved in 100 mL of purified water and stirred for 15 minutes. 5 syringes connected to gavage tubes are then each filled with 3 mL of the composition.

### Example 14

### Rectal Gels—Sodium Taurcholate Dose Response

### a) 50 mM Sodium Taurocholate

### Preparation Method:

Using a stainless steel dissolving vessel fitted with a propeller stirrer 2.688 grams of sodium taurocholate and 1 gram of methyl cellulose are dissolved in 100 mL of purified water and stirred for 15 minutes. 12 syringes connected to gavage tubes are then each filled with 3 mL of the composition.

### b) 150 mM Sodium Taurocholate

### Preparation Method:

Using a stainless steel dissolving vessel fitted with a propeller stirrer 8.066 grams of sodium taurocholate and 1 gram of methyl cellulose are dissolved in 100 mL of purified water and stirred for 15 minutes. 12 syringes connected to gavage tubes are then each filled with 3 mL of the composition.

### c) 500 mM Sodium Taurocholate

### Preparation Method:

Using a stainless steel dissolving vessel fitted with a propeller stirrer 26.88 grams of sodium taurocholate and 1 gram of methyl cellulose are dissolved in 100 mL of purified water and stirred for 15 minutes. 12 syringes connected to gavage tubes are then each filled with 3 mL of the composition.

### d) No Bile Salt (Control)

### Preparation Method:

Using a stainless steel dissolving vessel fitted with a propeller stirrer 1 gram of methyl cellulose is dissolved in 100 mL of purified water and stirred for 15 minutes. 12 syringes connected to gavage tubes are then each filled with 3 mL of the composition.

### Example 15

### Rectal Gels—Sodium Glycocholate/Control

### a) 500 mM Sodium Glycocholate

### Preparation Method:

Using a stainless steel dissolving vessel fitted with a propeller stirrer 24.38 grams of sodium glucocholate and 1 gram of methyl cellulose are dissolved in 100 mL of purified water and then stirred for 15 minutes. 6 syringes connected to gavage tubes are then each filled with 3 mL of the composition.

b) No Bile salt (control)

### Preparation Method:

Using a stainless steel dissolving vessel fitted with a propeller stirrer 1 gram of methyl cellulose is dissolved in 100 mL of purified water and stirred for 15 minutes. 5 syringes connected to gavage tubes are then each filled with 3 mL of the composition.

### Example 16

### Rectal Gels—Sodium Glycocholate Dose Response

### a) 50 mM Sodium Glycocholate

### Preparation Method:

Using a stainless steel dissolving vessel fitted with a propeller stirrer 2.44 grams of sodium glycocholate and 1 gram of methyl cellulose are dissolved in 100 mL of purified water and then stirred for 15 minutes. 12 syringes connected to gavage tubes are then each filled with 3 mL of the composition.

### b) 150 mM Sodium Glycocholate

### Preparation Method:

Using a stainless steel dissolving vessel fitted with a propeller stirrer 7.32 grams of sodium glycocholate and 1 gram of methyl cellulose are dissolved in 100 mL of purified water and then stirred for 15 minutes. 12 syringes connected to gavage tubes are then each filled with 3 mL of the composition.

### c) 500 mM Sodium Glycocholate

### Preparation Method:

Using a stainless steel dissolving vessel fitted with a propeller stirrer 24.38 grams of sodium glycocholate and 1 gram of methyl cellulose are dissolved in 100 mL of purified water and then stirred for 15 minutes. 12 syringes connected to gavage tubes are then each filled with 3 mL of the composition.

d) No Bile salt (control)

### Preparation Method:

Using a stainless steel dissolving vessel fitted with a propeller stirrer 1 gram of methyl cellulose is dissolved in 100 mL of purified water and then stirred for 15 minutes. 12 syringes connected to gavage tubes are then each filled with 3 mL of the composition.

### Example 17

### In Vivo Effects of Bile Acid, Taurocholate, on Plasma Active GLP-1 Levels in Pancreatitis Treatment

Reduction of pancreatic enzymes secretion is crucial factor for treatment of pancreatitis. GLP-1 reduces exocrine pancreatic secretion and has been demonstrated to improve and ameliorate pancreatitis. As such, it was our goal to increase plasma GLP-1 levels to decrease the markers of pancreatitis in human subjects.

Method:

Ten subjects were each studied on five separate occasions after an overnight fast and oral administration of 100 mg sitagliptin 10 hours before the study. The subjects then received an intrarectal infusion of either one of four doses of taurocholate (0.66, 2, 6.66, or 20 mmoles) or vehicle placebo in a random blinded fashion. Taurocholate was administered in 20 mL of a 1% carboxymethyl cellulose emulsion over 1 min. Plasma samples for GLP-1 hormone collected prior to, and for one hour following the infusion.

Results:

Taurocholate caused a dose-related increase of GLP-1, with 20 mmoles taurocholate resulting in peak concentrations -6 fold higher than placebo (P<0.0001). FIG. 2. ED₅₀ value for effects on integrated GLP-1 response was 8.1 mmoles.

Conclusion:

Rectally administered sodium taurocholate produced significant and dose-dependent increase of GLP-1 secretion, which is associated with treatment and prevention of pancreatitis. Enteroendocrine secretion enhancing agents such as taurocholate would be valuable in the treatment of pancreatitis.

### Example 18

### Enteric Coated Tablets

### a) 5 mg Sodium Taurocholate

### Preparation Method:

Preparation of core: 5 mg sodium taurocholate, 25 mg microcrystalline cellulose, 20 mg mannitol, and 10 mg croscarmellose sodium are mixed in a Hobart Mixer for 15 minutes. The mixture is granulated with 20% polyvinyl pyrrolidone (4 mg) solution until optimum granulation is obtained. The granulation is dried overnight at 50° C. The granulation is then passed through a #30 mesh. The granulation is then blended with 1 mg magnesium stearate. Using an F-Press ¼″ standard concave round punch, the granulation is compressed into a tablet. Preparation of erodible polymer layer and dual matrix tablets: 415 mg hydroxypropyl methylcellulose, 75 mg microcrystalline cellulose, and 6 mg polyvinylpyrrolidone are uniformly mixed with a mortar. The powder mix is granulated with 50% v/v alcohol solution until optimum granulation is obtained. The granulation is dried overnight at 50° C. The granulation is then passed through a #40 mesh screen. The granulation is then blended with 2.5 mg magnesium stearate. Using a Carver Press and a 7/16″ standard concave round punch, half of the granulation is placed in the die cavity, the core is then placed in the cavity and the other half of the granulation is placed in the die cavity. The mass is compressed to 5,000 lbs to form the dual matrix tablet. Enteric coating: Using a propellar mixer, 42 g of hydroxypropyl methylcellulose phthalate and 4.2 g of distilled acetylated monoglycerides are dissolved in 514 mL of a mixture of a cetone and absolute alcohol (1:1). Using a spray system, the dual matrix tablets are then coated with the enteric coating solution. Approximately 60 mg of the coating material (dry basis) is applied per tablet.

### b) 500 mM Sodium Glycocholate

### Preparation Method:

Preparation of core: 5 mg sodium glycocholate, 25 mg microcrystalline cellulose, 20 mg mannitol, and 10 mg croscarmellose sodium are mixed in a Hobart Mixer for 15 minutes. The mixture is granulated with 20% polyvinyl pyrrolidone (4 mg) solution until optimum granulation is obtained. The granulation is dried overnight at 50° C. The granulation is then passed through a #30 mesh. The granulation is then blended with 1 mg magnesium stearate. Using an F-Press ¼″ standard concave round punch, the granulation is compressed into a tablet. Preparation of erodible polymer layer and dual matrix tablets: 415 mg hydroxypropyl methylcellulose, 75 mg microcrystalline cellulose, and 6 mg polyvinylpyrrolidone are uniformly mixed with a mortar. The powder mix is granulated with 50% v/v alcohol solution until optimum granulation is obtained. The granulation is dried overnight at 50° C. The granulation is then passed through a #40 mesh screen. The granulation is then blended with 2.5 mg magnesium stearate. Using a Carver Press and a 7/16″ standard concave round punch, half of the granulation is placed in the die cavity, the core is then placed in the cavity and the other half of the granulation is placed in the die cavity. The mass is compressed to 5,000 lbs to form the dual matrix tablet. Enteric coating: Using a propellar mixer, 42 g of hydroxypropyl methylcellulose phthalate and 4.2 g of distilled acetylated monoglycerides are dissolved in 514 mL of a mixture of a cetone and absolute alcohol (1:1). Using a spray system, the dual matrix tablets are then coated with the enteric coating solution. Approximately 60 mg of the coating material (dry basis) is applied per tablet.

### c) No Bile Salt (Control)

### Preparation Method:

Preparation of core: 25 mg microcrystalline cellulose, 20 mg mannitol, and 10 mg croscarmellose sodium are mixed in a Hobart Mixer for 15 minutes. The mixture is granulated with 20% polyvinyl pyrrolidone (4 mg) solution until optimum granulation is obtained. The granulation is dried overnight at 50° C. The granulation is then passed through a #30 mesh. The granulation is then blended with 1 mg magnesium stearate. Using an F-Press ¼″ standard concave round punch, the granulation is compressed into a tablet. Preparation of erodible polymer layer and dual matrix tablets: 415 mg hydroxypropyl methylcellulose, 75 mg microcrystalline cellulose, and 6 mg polyvinylpyrrolidone are uniformly mixed with a mortar. The powder mix is granulated with 50% v/v alcohol solution until optimum granulation is obtained. The granulation is dried overnight at 50° C. The granulation is then passed through a #40 mesh screen. The granulation is then blended with 2.5 mg magnesium stearate. Using a Carver Press and a 7/16″ standard concave round punch, half of the granulation is placed in the die cavity, the core is then placed in the cavity and the other half of the granulation is placed in the die cavity. The mass is compressed to 5,000 lbs to form the dual matrix tablet. Enteric coating: Using a propellar mixer, 42 g of hydroxypropyl methylcellulose phthalate and 4.2 g of distilled acetylated monoglycerides are dissolved in 514 mL of a mixture of a cetone and absolute alcohol (1:1). Using a spray system, the dual matrix tablets are then coated with the enteric coating solution. Approximately 60 mg of the coating material (dry basis) is applied per tablet.

### Example 19

### Absorption Inhibitors

### a) Control: 500 mM Sodium Taurocholate

### Preparation Method:

Using a stainless steel dissolving vessel fitted with a propeller stirrer and turboemulsifier 26.88 grams of sodium taurocholate, 0.25 grams of potassium metabisulphite, 0.3 grams EDTA (disodium salt) and 0.38 grams of sodium benzoate dissolved in 100 mL of purified water. While stirring, 4 grams of Polysorbate 20 and 4 grams of Polyglycol 300 isostearate are added and stirring is continued for 15 minutes. The suspension is then pumped into an aerosol cans and is immediately sealed by clinching the dispenser valve. The can is then pressurized by pumping 6.5 grams of Freon 12 and 3.5 grams of Freon 114 into the can.

### b) 500 mM Sodium Taurocholate+Candidate Absorption Inhibitor

### Preparation Method:

Using a stainless steel dissolving vessel fitted with a propeller stirrer and turboemulsifier 26.88 grams of sodium taurocholate, 0.25 grams of potassium metabisulphite, 0.3 grams EDTA (disodium salt), 0.38 grams of sodium benzoate and between 0.01 grams and 20 grams of a candidate absorption inhibitor are dissolved in 100 mL of purified water. While stirring, 4 grams of Polysorbate 20 and 4 grams of Polyglycol 300 isostearate are added and stirring is continued for 15 minutes. The suspension is then pumped into an aerosol cans and is immediately sealed by clinching the dispenser valve. The can is then pressurized by pumping 6.5 grams of Freon 12 and 3.5 grams of Freon 114 into the can.

### Analysis of Absorption Inhibition

The foams described above are rectally administered to 5 conscious overnight-fasted subjects (e.g., Sprague Dawley rats). The ability of the absorption inhibitor to inhibit the absorption of the enteroendocrine peptide secretion enhancing agent (in this case sodium taurocholate) across the colon and/or rectum mucosa is determined by measuring the systemic concentration of enteroendocrine peptide secretion enhancing agent. Systemic concentration of enteroendocrine peptide secretion enhancing agent is measured prior to administration and at a time following administration of the enteroendocrine peptide secretion enhancing agent (e.g., after one hour). Decreased systemic concentration of the enteroendocrine peptide secretion enhancing agent indicate that the candidate absorption inhibitor inhibits the absorption of the enteroendocrine peptide secretion enhancing agent.

### Example 20

In certain instances, placing bile salts or other enteroendocrine peptide enhancing agents into the rectum has several advantages and provides substantial information on the whole process of releasing the distal gut hormones, GLP-2, oxyntomodulin and PYY. The studies include the following measurements:
Dose-responsive increase in GLP-2 and PYY levels in the bloodstream.
Elevation of high local concentrations of bile salt in the rectum without diarrhea.

### Example 21

### Clinical Trial to Test Efficacy of ASBTI in Treatment and/or Alleviation of Pancreatitis

This study will determine the efficacy of an ASBTI in treatment and/or alleviation of symptoms of pancreatitis.

Patients with a diagnosis of chronic pancreatitis based on imaging studies, persistent abdominal pain due to chronic pancreatitis, qualifying pain score during the pre-treatment period, and willing to comply with study visit schedule and study requirements are eligible.

Subjects will be administered a daily oral dose of compound 100B formulated for release in the distal ileum.

The primary endpoint is change from baseline in average chronic pancreatitis pain intensity score at 8 weeks. The secondary endpoint is change from baseline in average chronic pancreatitis pain intensity score at 16 weeks and change from baseline in worst chronic pancreatitis pain intensity score at 16 weeks.

Other ASBTIs, as well as in combination with an enteroendocrine peptide enhancing agent and/or FXR agonist, described herein can be tested in a clinical trial.

### Example 22

### Clinical Trial to Test Efficacy of Bile Acid Conjugate in Treatment and/or Alleviation of Symptoms of Pancreatitis

This study will determine efficacy of a bile acid conjugate for treatment in patients afflicted with pancreatitis.

Patients with a diagnosis of chronic pancreatitis based on imaging studies, persistent abdominal pain due to chronic pancreatitis, qualifying pain score during the pre-treatment period, and willing to comply with study visit schedule and study requirements are eligible.

Subjects will be administered a daily rectal dose of bile acid analog RG-239.

The primary endpoint is change from baseline in average chronic pancreatitis pain intensity score at 8 weeks. The secondary endpoint is change from baseline in average chronic pancreatitis pain intensity score at 16 weeks and change from baseline in worst chronic pancreatitis pain intensity score at 16 weeks.

Other enteroendocrine peptide enhancing agents, as well as in combination with an ASBTI and/or FXR agonist, described herein can be tested in a clinical trial.

### Example 23

### Clinical Trial to Test Efficacy of Fxr Agonist in Treatment and/or Alleviation of Symptoms of Pancreatitis

The purpose of this study is to determine the effect of FXR agonist suspension in treating pancreatitis

Patients with a diagnosis of chronic pancreatitis based on imaging studies, persistent abdominal pain due to chronic pancreatitis, qualifying pain score during the pre-treatment period, and willing to comply with study visit schedule and study requirements are eligible.

Subjects will be administered a daily dose of an FXR agonist suspension.

The primary endpoint is change from baseline in average chronic pancreatitis pain intensity score at 8 weeks. The secondary endpoint is change from baseline in average chronic pancreatitis pain intensity score at 16 weeks and change from baseline in worst chronic pancreatitis pain intensity score at 16 weeks.

Other FXR agonists, as well as in combination with an ASBTI and/or enteroendocrine peptide enhancing agent, described herein can be tested in a clinical trial.

1. A method for the treatment of pancreatitis in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

2. A method for the treatment of pain associated with pancreatitis in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

3. A method for prevention of acute or chronic pancreatitis in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

4. The method of claim 3, wherein the individual has undergone a surgical pancreato-biliary intervention or procedure.

5. The method of claim 4, wherein the surgical pancreato-biliary intervention or procedure is pancreatic resection, Endoscopic Retrograde Cholangiopancreatography Procedure (ERCP), gallbladder surgery, bile duct surgery, liver surgery, liver transplantation, or bariatric surgery.

6. A method for increasing the levels of a pancreatic peptide or hormone or an enteroendocrine peptide or hormone in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

7. The method of claim 6, wherein the pancreatic peptide or hormone is amylin or insulin.

8. The method of claim 6, wherein the enteroendocrine peptide or hormone is glucagon-like peptide 1 (GLP-1), GLP-2, peptide tyrosine-tyrosine (PYY), or oxyntomodulin (OXM).

9. A method for decreasing the levels of a pancreatic enzyme in an individual in need thereof comprising non-systemically administering to the individual in need thereof a therapeutically effective amount of an Apical Sodium-dependent Bile Acid Transporter Inhibitor (ASBTI) or a pharmaceutically acceptable salt thereof, an enteroendocrine peptide enhancing agent or a pharmaceutically acceptable salt thereof, or an FXR agonist or a pharmaceutically acceptable salt thereof, or a combination thereof.

10. The method of claim 9, wherein the pancreatic peptide or hormone is amylase or lipase.

11. The method of claim 1, further comprising administration of a second agent selected from a liver receptor homolog 1 (LRH-1), a DPP-IV inhibitor, a proton pump inhibitor, H2 antagonist, prokinetic agent, a biguanide, an incretin mimetic, a mucoadhesive agent, GLP-1 or an analog thereof, and a pancreatic enzyme.

12. The method of claim 1, further comprising administration of a pain relieving medication.

13. The method of claim 1, wherein the non-systemically administered Apical Sodium-dependent Bile Acid Transporter Inhibitor (ASBTI), enteroendocrine peptide enhancing agent, or nuclear farnesoid X receptor (FXR) agonist reduces inflammation and/or damage to pancreas in an individual in need thereof.

14. The method of claim 1, wherein the ASBTI is a compound of Formula I: 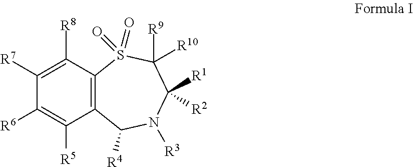
wherein:
R¹ is a straight chained C₁₋₆ alkyl group;
R² is a straight chained C₁₋₆alkyl group;
R³ is hydrogen or a group OR¹¹ in which R¹¹ is hydrogen, optionally substituted C₁₋₆alkyl or a C₁₋₆ alkylcarbonyl group;
R⁴ is pyridyl or optionally substituted phenyl or -L_{z}-K_{z}; wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
R⁵, R⁶, R⁷ and R⁸ are the same or different and each is selected from hydrogen, halogen, cyano, R⁵-acetylide, OR¹⁵, optionally substituted C₁₋₆alkyl, COR¹⁵, CH(OH)R¹⁵, S(O)ₙ, R⁵, P(O)(OR¹⁵)₂, OCOR¹⁵, OCF3, OCN, SCN, NHCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂RS, OCH₂OR¹⁵, OCH═CHR¹⁵, O(CH₂CH₂O)ₙR¹⁵, O(CH₂)ₚSO₃R¹⁵, O(CH₂)ₚNR¹²R¹³, O(CH₂)ₚN⁺R¹²R¹³R¹⁴ and —W—R³¹, wherein W is O or NH, and R³¹ is selected from 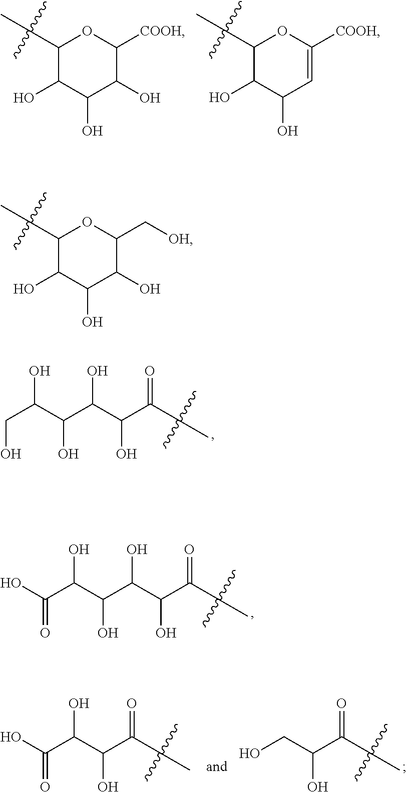
wherein p is an integer from 1-4, n is an integer from 0-3 and, R¹², R¹³, R¹⁴ and R¹⁵ are independently selected from hydrogen and optionally substituted C₁₋₆ alkyl; or
R⁶ and R⁷ are linked to form a group 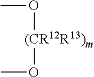
wherein R¹² and R¹³ are as hereinbefore defined and m is 1 or 2; and
R⁹ and R¹⁰ are the same or different and each is selected from hydrogen or C₁₋₆alkyl; and salts, solvates and physiologically functional derivatives thereof.

15. The method of claim 11, wherein the compound of Formula I is

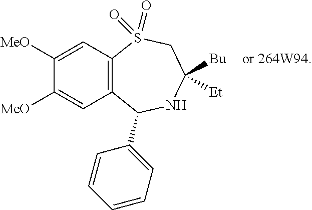

16. The method of claim 1, wherein the ASBTI is a compound of Formula II: 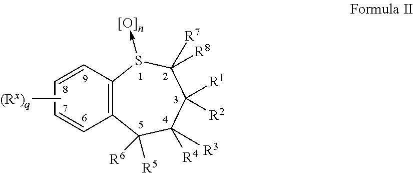
wherein:
q is an integer from 1 to 4;
n is an integer from 0 to 2;
R¹ and R² are independently selected from the group consisting of H, alkyl, alkenyl, alkynyl, haloalkyl, alkylaryl, arylalkyl, alkoxy, alkoxyalkyl, dialkylamino, alkylthio, (polyalkyl)aryl, and cycloalkyl, wherein alkyl, alkenyl, alkynyl, haloalkyl, alkylaryl, arylalkyl, alkoxy, alkoxyalkyl, dialkylamino, alkylthio, (polyalkyl)aryl, and cycloalkyl optionally are substituted with one or more substituents selected from the group consisting of OR⁹, NR⁹R¹⁰, N⁺R⁹R¹⁰R^{w}A⁻, SR⁹, S⁺R⁹R¹⁰A⁻, P⁺R⁹R¹⁰R¹¹A⁻, S(O)R⁹, SO₂R⁹, SO₃R⁹, CO₂R⁹, CN, halogen, oxo, and CONR⁹R¹⁰,
wherein alkyl, alkenyl, alkynyl, alkylaryl, alkoxy, alkoxyalkyl, (polyalkyl)aryl, and cycloalkyl optionally have one or more carbons replaced by O, NR⁹, N⁺R⁹R¹⁰A⁻, S, SO, SO₂, S⁺R⁹A⁻, P⁺R⁹R¹⁰A⁻, or phenylene,
wherein R⁹, R¹⁰, and R^{w} are independently selected from the group consisting of H, alkyl, alkenyl, alkynyl, cycloalkyl, aryl, acyl, heterocycle, ammoniumalkyl, arylalkyl, and alkylammoniumalkyl; or
R¹ and R² taken together with the carbon to which they are attached form C₃-C₁₀cycloalkyl;
R³ and R⁴ are independently selected from the group consisting of H, alkyl, alkenyl, alkynyl, acyloxy, aryl, heterocycle, OR⁹, NR⁹R¹⁰, SR⁹, S(O)R⁹, SO₂R⁹, and SO₃R⁹, wherein R⁹ and R¹⁰ are as defined above; or
R³ and R⁴ together ═O, ═NOR¹¹, ═S, ═NNR¹¹R¹², ═NR⁹, or ═CR¹¹R¹² wherein R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R⁵ and R⁶ are independently selected from the group consisting of H, alkyl, alkenyl, alkynyl, aryl, cycloalkyl, heterocycle, quaternary heterocycle, quarternary heteroaryl, OR³⁰, SR⁹, S(O)R⁹, SO₂R⁹, SO₃R⁹, and -L_{z}-K_{z};
wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
wherein alkyl, alkenyl, alkynyl, aryl, cycloalkyl, heterocycle, quaternary heterocycle, and quaternary heteroaryl can be substituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂ NR¹³R¹⁴, C(O)NR¹³R¹⁴, C(O)OM, CR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻,
wherein:
A⁻ is a pharmaceutically acceptable anion and M is a pharmaceutically acceptable cation, said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, S(O)R⁷, SO₂R⁷, SO₃R⁷, CO₂R⁷, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(O)(OR⁷) OR⁸ and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene, and R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, and quaternary heteroarylalkyl,
wherein alkyl, alkenyl, alkynyl, arylalkyl, heterocycle, and polyalkyl optionally have one or more carbons replaced by O, NR⁹, N⁺R⁹R¹⁰A⁻, S, SO, SO₂, S⁺R⁹A⁻, PR, P⁺R⁹R¹⁰A⁻, P(O)R⁹, phenylene, carbohydrate, amino acid, peptide, or polypeptide, and
R¹³, R¹⁴ and R¹⁵ are optionally substituted with one or more groups selected from the group consisting of sulfoalkyl, quaternary heterocycle, quaternary heteroaryl, OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂ NR⁹R¹⁰, PO(OR¹⁶)OR¹⁷, P⁺R⁹R¹⁰R¹¹A⁻, S⁺R⁹R¹⁰A⁻, and C(O)OM,
wherein R¹⁶ and R¹⁷ are independently selected from the substituents constituting R⁹ and M; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl; and
R⁷ and R⁸ are independently selected from the group consisting of hydrogen and alkyl; and one or more R^{x} are independently selected from the group consisting of H, alkyl, alkenyl, alkynyl, polyalkyl, acyloxy, aryl, arylalkyl, halogen, haloalkyl, cycloalkyl, heterocycle, heteroaryl, polyether, quaternary heterocycle, quaternary heteroaryl, OR¹³, NR¹³R¹⁴, SR¹³, S(O)R¹³, S(O)₂R³, SO₃R¹³, S⁺R¹³R¹⁴A⁻, NR¹³OR¹⁴, NR¹³NR¹⁴R^{1s}, NO₂, CO₂R¹³, CN, OM, SO₂OM, SO₂ NR¹³R¹⁴, NR¹⁴C(O)R¹³, C(O)NR¹³R¹⁴ NR¹⁴C(O)R¹³, C(O)OM, COR¹³, OR¹⁸, S(O)ₙNR¹⁸, NR¹³R¹⁸, NR¹⁸R¹⁴, N⁺12⁹R¹¹R¹²A⁻, P⁺R⁹R¹¹R¹²A⁻, amino acid, peptide, polypeptide, and carbohydrate;
wherein alkyl, alkenyl, alkynyl, cycloalkyl, aryl, polyalkyl, heterocycle, acyloxy, arylalkyl, haloalkyl, polyether, quaternary heterocycle, and quaternary heteroaryl can be further substituted with OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂ NR⁹R¹⁰, PO(OR¹⁶)OR¹⁷P⁺R⁹R¹¹R¹²A⁻, S⁺R⁹R¹⁰A⁻, or C(O)M,
wherein W is O or NH, R³¹ is selected from
wherein R¹⁸ is selected from the group consisting of acyl, arylalkoxycarbonyl, arylalkyl, heterocycle, heteroaryl, alkyl,
wherein acyl, arylalkoxycarbonyl, arylalkyl, heterocycle, heteroaryl, alkyl, quaternary heterocycle, and quaternary heteroaryl optionally are substituted with one or more substituents selected from the group consisting of OR⁹, NR⁹R¹⁰, N⁺R⁹R¹¹R¹²A⁻, SR⁹, S(O)R⁹, SO₂R⁹, SO₃R⁹, oxo, CO₃R⁹, CN, halogen, CONR⁹R¹⁰, SO₃R⁹, SO₂OM, SO₂ NR⁹R¹⁰, PO(OR¹⁶)OR⁷, and C(O)OM,
wherein in R^{x}, one or more carbons are optionally replaced by O, NR¹³, N⁺R¹³R¹⁴A⁻, S, SO, SO₂, S⁺R¹³A⁻, PR¹³, P(O)R¹³, P⁺R¹³R¹⁴A⁻, phenylene, amino acid, peptide, polypeptide, carbohydrate, polyether, or polyalkyl,
wherein in said polyalkyl, phenylene, amino acid, peptide, polypeptide, and carbohydrate, one or more carbons are optionally replaced by O, NR⁹, R⁹R¹⁰A⁻, S, SO, SO₂, S⁺R⁹A⁻, PR⁹, P⁺R⁹R¹⁰A⁻, or P(O)R⁹;
wherein quaternary heterocycle and quaternary heteroaryl are optionally substituted with one or more groups selected from the group consisting of alkyl, alkenyl, alkynyl, polyalkyl, polyether, aryl, haloalkyl, cycloalkyl, heterocycle, arylalkyl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R³, CN, OM, SO₂OM, SO₂ NR¹³R¹⁴, C(O)NR¹³R¹⁴, C(O)OM, COR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹R¹²A⁻,
provided that both R⁵ and R⁶ cannot be hydrogen or SH;
provided that when R⁵ or R⁶ is phenyl, only one of R¹ or R² is H;
provided that when q=1 and R^{x} is styryl, anilido, or anilinocarbonyl, only one of R⁵ or R⁶ is alkyl; or a
pharmaceutically acceptable salt, solvate, or prodrug thereof

17. The method of claim 16, wherein:
q is an integer from 1 to 4;
n is 2;
R¹ and R² are independently selected from the group consisting of H, alkyl, alkoxy, dialkylamino, and alkylthio,
wherein alkyl, alkoxy, dialkylamino, and alkylthio are optionally substituted with one or more substituents selected from the group consisting of OR⁹, NR⁹R¹⁰, SR⁹, SO₂R⁹, CO₂R⁹, CN, halogen, oxo, and CONR⁹R¹⁰;
each R⁹ and R¹⁰ are each independently selected from the group consisting of H, alkyl, cycloalkyl, aryl, acyl, heterocycle, and arylalkyl;
R³ and R⁴ are independently selected from the group consisting of H, alkyl, acyloxy, OR⁹, NR⁹R¹⁰, SR⁹, and SO₂R⁹, wherein R⁹ and R¹⁰ are as defined above;
R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R⁵ and R⁶ are independently selected from the group consisting of H, alkyl, aryl, cycloalkyl, heterocycle, and -L_{z}-K_{z};
wherein z is 1 or 2; each L is independently a substituted or unsubstituted alkyl, a substituted or unsubstituted heteroalkyl, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
wherein alkyl, aryl, cycloalkyl, and heterocycle can be substituted with one or more substituent groups independently selected from the group consisting of alkyl, aryl, haloalkyl, cycloalkyl, heterocycle, arylalkyl, quaternary heterocycle, quaternary heteroaryl, halogen, oxo, OR¹³, NR¹³R¹⁴, SR¹³, SO₂R¹³, NR¹³NR¹⁴R¹⁵, NO₂, CO₂R¹³, CN, OM, and CR¹³,
wherein:
A⁻ is a pharmaceutically acceptable anion and M is a pharmaceutically acceptable cation;
R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, and quaternary heteroarylalkyl, wherein R¹³, R¹⁴ and R¹⁵ are optionally substituted with one or more groups selected from the group consisting of quaternary heterocycle, quaternary heteroaryl, OR⁹, NR⁹R¹⁰, N⁺R⁹R¹¹R¹²A⁻, SR⁹, S(O)R⁹, SO₂R⁹, SO₃R⁹, Oxo, CO₂R⁹, CN, halogen, and CONR⁹R¹⁰; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl; and
R⁷ and R⁸ are independently selected from the group consisting of hydrogen and alkyl; and one or more R^{x} are independently selected from the group consisting of H, alkyl, acyloxy, aryl, arylalkyl, halogen, haloalkyl, cycloalkyl, heterocycle, heteroaryl, OR¹³, NR¹³R¹⁴, SR¹³, S(O)₂R¹³, NR¹³NR¹⁴R^{1s} NO₂, CO₂R¹³, CN, SO₂ NR¹³R¹⁴, NR¹⁴C(O)R¹³, C(O)NR¹³R¹⁴, NR¹⁴C(O)R¹³, and COR¹³;
provided that both R⁵ and R⁶ cannot be hydrogen;
provided that when R⁵ or R⁶ is phenyl, only one of R¹ or R² is H;
provided that when q=1 and R^{x} is styryl, anilido, or anilinocarbonyl, only one of R⁵ or R⁶ is alkyl; or a pharmaceutically acceptable salt, solvate, or prodrug thereof.

18. The method of claim 16, wherein the compound of Formula II is 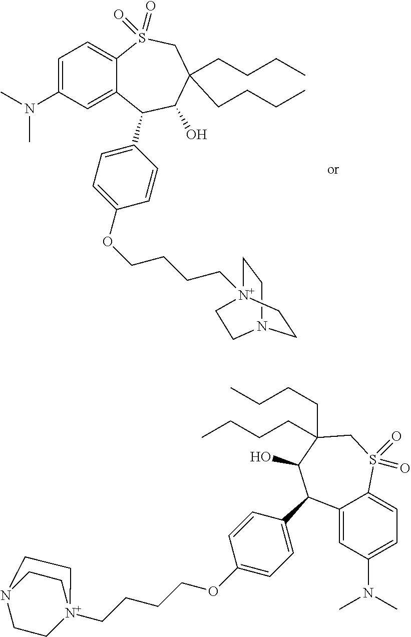 [SMILES: CCCCC1(CCCC)CS(=O)(=O)c2ccc(N(C)C)cc2[C@@H](c2ccc(OCCCC[N+]34CCN(CC3)CC4)cc2)[C@H]1O ; CCCCC1(CCCC)CS(=O)(=O)c2ccc(N(C)C)cc2[C@@H](c2ccc(OCCCC[N+]34CCN(CC3)CC4)cc2)[C@H]1O]
or a salt thereof.

19. The method of claim 16, wherein the compound of Formula II is 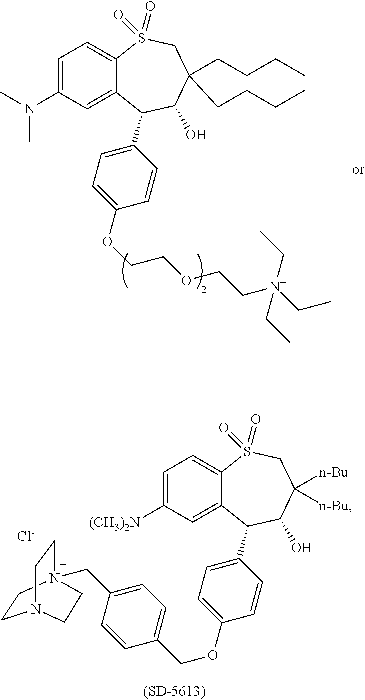
optionally further comprising sitagliptin.

20. The method of claim 16, wherein the compound of Formula II is

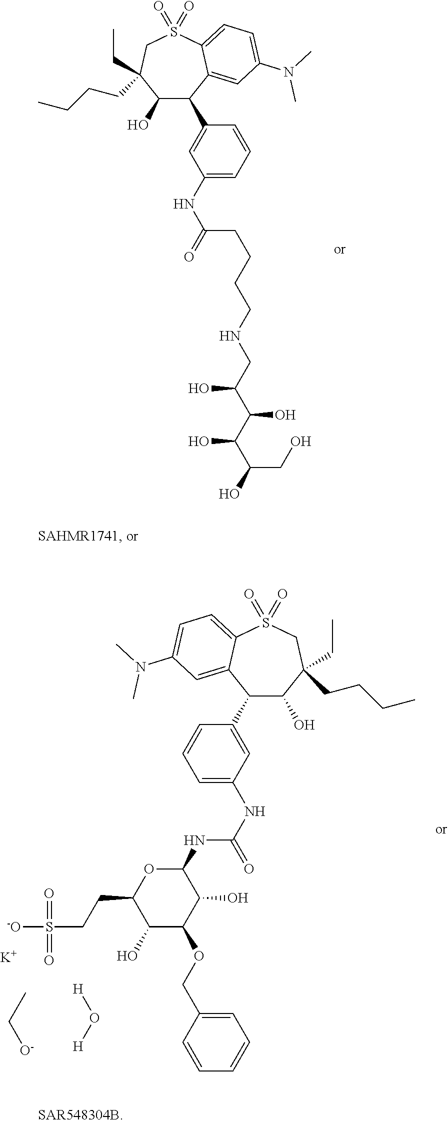

21. The method of claim 1, wherein the ASBTI is a compound of Formula III: 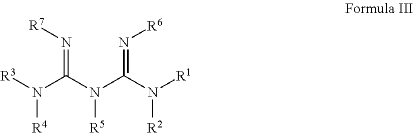
wherein:
each R¹, R² is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R¹ and R² together with the nitrogen to which they are attached form a 3-8-membered ring that is optionally substituted with R⁸;
each R³, R⁴ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
R⁵ is H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl,
each R⁶, R⁷ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R⁶ and R⁷ taken together form a bond;
each X is independently NH, S, or O;
each Y is independently NH, S, or O;
R⁸ is substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
L is Aₙ, wherein
each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
n is 0-7;
K is a moiety that prevents systemic absorption;
provided that at least one of R¹, R², R³ or R⁴ is -L-K;
or a pharmaceutically acceptable prodrug thereof.

22. The method of claim 1, wherein the ASBTI is a compound of Formula IV: 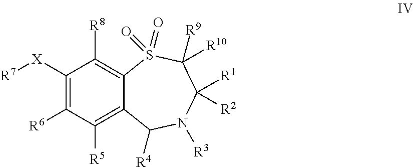
wherein
R¹ is a straight chain C₁₋₆alkyl group;
R² is a straight chain C₁₋₆alkyl group;
R³ is hydrogen or a group OR¹¹ in which R¹¹ is hydrogen, optionally substituted C₁₋₆ alkyl or a C_{—-6} alkylcarbonyl group;
R⁴ is pyridyl or an optionally substituted phenyl;
R⁵, R⁶ and R⁸ are the same or different and each is selected from:
hydrogen, halogen, cyano, R¹⁵-acetylide, OR¹⁵, optionally substituted C₁₋₆ alkyl, COR¹⁵, CH(OH)R^{S}1, S(O)ₙR¹⁵, P(O)(OR¹⁵)₂, OCOR¹⁵, OCF₃, OCN, SCN, NHCN, CH₂OR⁵, CHO, (CH₂)ₚCN, CONR¹²R¹³, (CH₂)ₚCO₂R¹⁵, (CH₂)ₚNR¹²R¹³, CO₂R⁵, NHCOCF₃, NHSO₂R⁵, OCH₂OR^{S}1, OCH═CHR¹⁵, O(CH₂CH₂O)R⁵, O(CH₂)ₚSO₃R⁵, O(CH₂)ₚNR¹²R¹³ and O(CH₂)ₚN⁺R¹²R¹³R¹⁴ wherein
p is an integer from 1-4,
n is an integer from 0-3 and
R¹², R¹³, R¹⁴ and R^{s}1 are independently selected from hydrogen and optionally substituted C₁₋₆alkyl;
R⁷ is a group of the formula 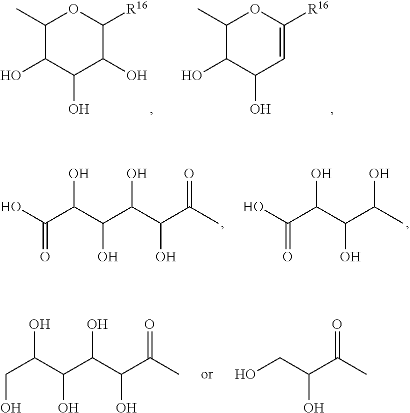
wherein the hydroxyl groups may be substituted by acetyl, benzyl, or
—(C₁-C₆)-alkyl-R¹⁷,
wherein the alkyl group may be substituted with one or more hydroxyl groups;
R¹⁶ is —COOH, —CH₂—OH, —CH₂—O-Acetyl, —COOMe or —COOEt;
R¹⁷ is H, —OH, —NH₂, —COOH or COOR¹⁸;
R¹⁸ is (C₁-C₄)-alkyl or —NH—(C₁-C₄)-alkyl;
X is —NH— or —O—; and
R⁹ and R¹⁰ are the same or different and each is hydrogen or C₁-C₆alkyl; and salts thereof.

23. The method of claim 1, wherein the ASBTI is a compound of Formula V: 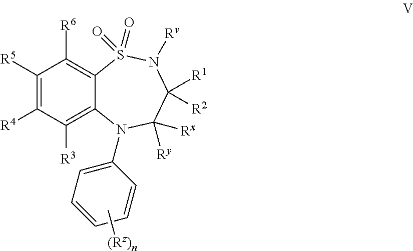
wherein: R^{v} is selected from hydrogen or C₁₋₆alkyl;
One of R¹ and R² are selected from hydrogen or C₁₋₆alkyl and the other is selected from C₁₋₆alkyl;
R^{x} and R^{y} are independently selected from hydrogen, hydroxy, amino, mercapto, C₁₋₆alkyl, C₁₋₆alkoxy, N—(C₁₋₆alkyl)amino, N,N—(C₁₋₆alkyl)₂amino, C₁₋₆alkylS(O)ₐ wherein a is 0 to 2;
R^{z} is selected from halo, nitr, cyano, hydroxy, amino, carboxy, carbamoyl, mercapto, sulphamoyl, C₁₋₆ alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆-alkyl)sulphamoyl and N,N—(C₁₋₆alkyl)₂sulphamoyl;
n is 0-5;
one of R⁴ and R^{s} is a group of formula (VA): 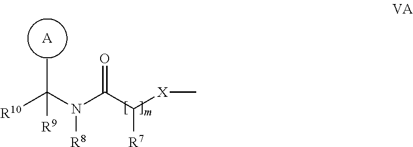
R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆ alkoxy, C₁₋₆alkanoyl, C₁₋₆alkanoyloxy, N—(C₁₋₆alkyl)amino, N,N—(C₁₋₆alkyl)₂amino, C₁₋₆ alkanoylamino, N—(C₁₋₆alkyl)carbamoyl, N,N—(C₁₋₆alkyl)₂carbamoyl, C₁₋₆alkylS(O)ₐ, wherein a is 0 to 2, C₁₋₆alkoxycarbonyl, N—(C₁₋₆alkyl)sulphamoyl and N,N—(C₁₋₆alkyl)₂sulphamoyl;
wherein R³ and R⁶ and the other of R⁴ and R⁵ may be optionally substituted on carbon by one or more R¹⁷;
X is —O—, —N(R^{a})—, —S(O)_{b}— or —CH(R^{a})—;
wherein R^{a} is hydrogen or C₁₋₆alkyl and b is 0-2;
Ring A is aryl or heteroaryl;
wherein Ring A is optionally substituted on carbon by one or more substituents selected from R¹⁸;
R⁷ is hydrogen, C₁₋₆alkyl, carbocyclyl or heterocyclyl;
wherein R⁷ is optionally substituted on carbon by one or more substituents selected from R¹⁹; and wherein if said heterocyclyl contains an —NH— group, that nitrogen may be optionally substituted by a group selected from R²⁰;
R⁸ is hydrogen or C₁₋₆-alkyl;
R⁹ is hydrogen or C₁₋₆alkyl;
R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkynyl, C₂₋₁₀alkynyl, C₁₋₁₀alkoxy, C₁₋₁₀ alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²¹—(C₁₋₁₀alkylene)_{q}- or heterocyclyl-(C₁₋₁₀alkylene)ᵣ-R²²—(C₁₋₁₀alkylene)ₛ-; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (VB): 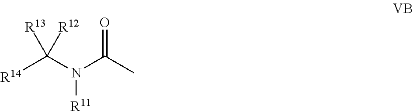
wherein:
R¹¹ is hydrogen or C₁₋₆-alkyl;
R¹² and R¹³ are independently selected from hydrogen, halo, carbamoyl, sulphamoyl, C₁₋₁₀alkyl, C₂₋₁₀alkynyl, C₂₋₁₀alkynyl, C₁₋₁₀alkanoyl, N—(C₁₋₁₀alkyl)carbamoyl, N,N—(C₁₋₁₀alkyl)₂carbamoyl, C₁₋₁₀ alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;
R¹⁴ is selected from hydrogen, halo, carbamoyl, sulphamoyl, hydroxyaminocarbonyl, C₁₋₁₀alkyl, C₂₋₁₀alkenyl, C₂₋₁₀alkynyl, C₁₋₁₀alkanoyl, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene), —R²⁷—(C₁₀ q- or heterocyclyl-(C₁₋₁₀alkylene), —R²⁸—(C₁₋₁₀alkylene)ₛ-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (VC): 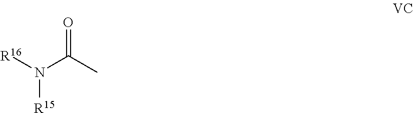
R¹⁵ is hydrogen or C₁₋₆alkyl; and R¹⁶ is hydrogen or C₁₋₆alkyl; wherein R¹⁶ may be optionally substituted on carbon by one or more groups selected from R³¹;
or R¹⁵ and R¹⁶ together with the nitrogen to which they are attached form a heterocyclyl; wherein said heterocyclyl may be optionally substituted on carbon by one or more R³⁷; and wherein if said heterocyclyl contains an —NH— group, that nitrogen may be optionally substituted by a group selected from R³⁸;
m is 1-3; wherein the values of R⁷ may be the same or different;
R¹⁷, R¹⁸, R¹⁹, R²³, R²⁵, R²⁹, R³⁰ and R³⁷ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ, wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)-R³³—(C₁₋₁₀alkylene)ₛ-; wherein R¹⁷, R¹⁸, R¹⁹, R²³, R²⁵, R²⁹, R³¹ and R³⁷ may be independently optionally substituted on carbon by one or more R³⁴; and wherein if said heterocyclyl contains an —NH— group, that nitrogen may be optionally substituted by a group selected from R³⁵;
R²¹, R²², R²⁷, R²⁸, R³² or R³³ are independently selected from —O—, —NR³⁶—, —S(O)ₓ—, NR³⁶C(O)NR³⁶—, —NR³⁶C(S)NR³⁶—, —OC(O)N═C—, —NR³⁶C(O)— or —C(O)NR³⁶—;
wherein R³⁶ is selected from hydrogen or C₁₋₆alkyl, and x is 0-2; p, q, r and s are independently selected from 0-2;
R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, allyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulpharoyl, N-methylsulphamoylamino and N,N-dimethylsulphamoylamino;
R²⁰, R²⁴, R²⁶, R³⁰, R³⁵ and R³⁸ are independently selected from C₁₋₆alkyl, C₁₋₆alkanoyl, C₁₋₆ alkylsulphonyl, C₁₋₆ alkoxycarbonyl, carbamoyl, N—(C₁₋₆alkyl)carbamoyl, N,N—(C₁₋₆ alkyl)carbamoyl, benzyl, benzyloxycarbonyl, benzoyl and phenylsulphonyl; and
wherein a “heteroaryl” is a totally unsaturated, mono or bicyclic ring containing 3-12 atoms of which at least one atom is chosen from nitrogen, sulphur and oxygen, which heteroaryl may, unless otherwise specified, be carbon or nitrogen linked;
wherein a “heterocyclyl” is a saturated, partially saturated or unsaturated, mono or bicyclic ring containing 3-12 atoms of which at least one atom is chosen from nitrogen, sulphur and oxygen, which heterocyclyl may, unless otherwise specified, be carbon or nitrogen linked, wherein a —CH₂-group can optionally be replaced by a —C(O)— group, and a ring sulphur atom may be optionally oxidised to form an S-oxide; and
wherein a “carbocyclyl” is a saturated, partially saturated or unsaturated, mono or bicyclic carbon ring that contains 3-12 atoms; wherein a —CH₂— group can optionally be replaced by a —C(O) group;
or a pharmaceutically acceptable salt or in vivo hydrolysable ester or amide formed on an available carboxy or hydroxy group thereof.

24. The method of claim 1, wherein the ASBTI is a compound of Formula VI: 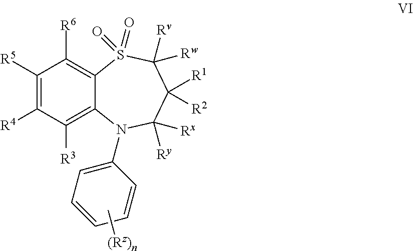
wherein:
R^{v} and R^{w} are independently selected from hydrogen or C₁₋₆alkyl;
one of R¹ and R² is selected from hydrogen or C₁₋₆alkyl and the other is selected from C₁₋₆alkyl;
R^{x} and R^{y} are independently selected from hydrogen or C₁₋₆alkyl, or one of R^{x} and R^{y} is hydrogen or C₁₋₆alkyl and the other is hydroxy or C₁₋₆alkoxy;
R^{z} is selected from halo, nitro, cyano, hydroxy, amino, carboxy, carbamoyl, mercapto, sulphamoyl, C₁₋₆alkyl, C₂₋₆ alkenyl, C₂₋₆alkynyl, C₁₋₆alkoxy, C₁₋₆ alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl and N,N—(C₁₋₆alkyl)₂sulphamoyl;
n is 0-5;
one of R⁴ and R⁵ is a group of formula (VIA): 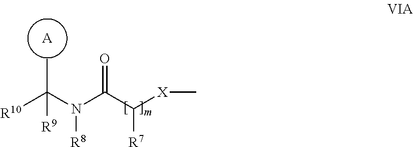
R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆ alkoxy, C₁₋₆alkanoyl, C₁₋₆alkanoyloxy, N—(C₁₋₆alkyl)amino, N,N—(C₁₋₆alkyl)₂amino, C₁₋₆ alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆ alkoxycarbonyl, N—(C₁₋₆alkyl)sulphamoyl and N,N—(C₁₋₆alkyl)₂sulphamoyl; wherein R³ and R⁶ and the other of R⁴ and R⁵ may be optionally substituted on carbon by one or more R⁷;
X is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted on carbon by one or more substituents selected from R¹⁸;
R⁷ is hydrogen, C₁₋₆alkyl, carbocyclyl or heterocyclyl; wherein R⁷ is optionally substituted on carbon by one or more substituents selected from R¹⁹; and wherein if said heterocyclyl contains an —NH— group, that nitrogen may be optionally substituted by a group selected from R²⁰;
R⁸ is hydrogen or C₁₋₆alkyl;
R⁹ is hydrogen or C₁₋₆alkyl;
R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀ alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)-R²¹—(C₁₋₁₀alkylene)_{q}- or heterocyclyl-(C₁₋₁₀alkylene)ᵣ-R²²—(C₁₋₁₀alkylene)ₛ-; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (VIB): 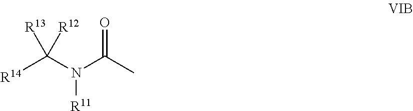
wherein:
R¹¹ is hydrogen or C₁₋₆alkyl;
R¹² and R¹³ are independently selected from hydrogen, halo, nitro, cyano, hydroxy, amino, carbamoyl, mercapto, sulphamoyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;
R¹⁴ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O), wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷—(C₁₋₁₀alkylene)_{q}- or heterocyclyl-(C₁₋₁₀alkylene)ᵣ-R²⁸—(C₁₋₁₀alkylene)ₛ-; wherein R¹⁴ may be optionally substituted on carbon by one or more substituents selected from R²⁹; and
wherein if said heterocyclyl contains an —NH— group, that nitrogen may be optionally substituted by a group selected from R³⁰; or R¹⁴ is a group of formula (VIC): 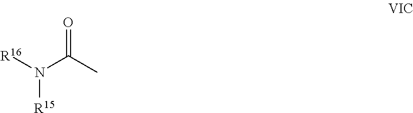
R¹⁵ is hydrogen or C₁₋₆alkyl;
R¹⁶ is hydrogen or C₁₋₆alkyl; wherein R¹⁶ may be optionally substituted on carbon by one or more groups selected from R³¹;
n is 1-3; wherein the values of R⁷ may be the same or different;
R¹⁷, R¹⁸, R¹⁹, R²³, R²⁵, R²⁹ or R³¹ are independently selected from halo, nitro, cyano, hydroxy, amino, carbamoyl, mercapto, sulphamoyl, hydroxyaminocarbonyl, amidino, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, (C₁₋₁₀alkyl)₃ silyl, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)- or heterocyclyl-(C₁₋₁₀alkylene)ᵣ-R³³—(C₁₋₁₀alkylene)ₛ-; wherein R¹⁷, R¹⁸, R¹⁹, R²³, R²⁵, R²⁹ or R³¹ may be independently optionally substituted on carbon by one or more R³⁴; and wherein if said heterocyclyl contains an —NH— group, that nitrogen may be optionally substituted by a group selected from R³⁵;
R²¹, R²², R²⁷, R²⁸, R³² or R³ are independently selected from —O—, —NR³⁶—, —S(O)ₓ, —NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;
p, q, r and s are independently selected from 0-2;
R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, allyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;
R²⁰, R²⁴, R²⁶, R³⁰ or R³⁵ are independently selected from C₁₋₆alkyl, C₁₋₆ alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
or a pharmaceutically acceptable salt, solvate or solvate of such a salt, or an in vivo hydrolysable ester formed on an available carboxy or hydroxy thereof, or an in vivo hydrolysable amide formed on an available carboxy thereof.

25. The method of claim 1, wherein the enteroendocrine peptide enhancing agent is a bile acid, a bile salt, a bile acid mimic, a bile salt mimic, or a combination thereof.

26. The method of claim 25, wherein the bile acid or the bile acid mimic is a compound represented by Formula (VII): 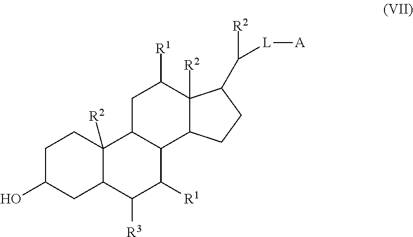
wherein:
each R¹ is independently H, OH, lower alkyl, or lower heteroalkyl;
L is a substituted or unsubstituted alkyl or substituted or unsubstituted heteroalkyl;
each R² is independently H, OH, lower alkyl, or lower heteroalkyl;
R³ is H, OH, O-lower alkyl, lower alkyl, or lower heteroalkyl;
A is COOR⁴, S(O)ₙR⁴, or OR⁵;
R⁴ is H, an anion, a pharmaceutically acceptable cation, substituted or unsubstituted alkyl, substituted or unsubstituted heteroalkyl, substituted or unsubstituted aryl, substituted or unsubstituted heteroaryl, or an amino acid;
n is 1-3;
R⁵ is lower alkyl or H.

27. The method of claim 26, wherein the bile acid mimic is a TGR5-binding analog, M-BAR agonist, GPR119 agonist, GPR120 agonist, GPR131 agonist, GPR140 agonist, GPR143 agonist, GPR53 agonist, GPBAR1 agonist, BG37 agonist, FXR agonist, 6-methyl-2-oxo-4-thiophen-2-yl-1,2,3,4-tetrahydro-pyrimidine-5-carboxylic acid benzyl ester, INT-777, RG-239, oleanolic acid, or crataegolic acid.

28. The method of claim 26, wherein the bile acid is a cholic acid, a deoxycholic acid, a glycocholic acid, a glycodeoxycholic acid, a taurocholic acid, a taurodihydrofusidate, a taurodeoxycholic acid, a cholate, a glycocholate, a deoxycholate, a taurocholate, a taurodeoxycholate, a chenodeoxycholic acid, an ursodeoxycholic acid, a tauroursodeoxycholic acid, a glycoursodeoxycholic acid, a 7-B-methyl cholic acid, a methyl lithocholic acid, or a salt thereof, or a combination thereof.

29. The method of claim 1, wherein the FXR agonist is GW4064, GW9662, INT-747, T0901317, WAY-362450, fexaramine, a cholic acid, a deoxycholic acid, a glycocholic acid, a glycodeoxycholic acid, a taurocholic acid, a taurodihydrofusidate, a taurodeoxycholic acid, a cholate, a glycocholate, a deoxycholate, a taurocholate, a taurodeoxycholate, a chenodeoxycholic acid, an ursodeoxycholic acid, a tauroursodeoxycholic acid, a glycoursodeoxycholic acid, a 7-B-methyl cholic acid, a methyl lithocholic acid, or a salt thereof, or a combination thereof.

30. The method of claim 1, wherein the ASBTI and/or the enteroendocrine peptide enhancing agent and/or the FXR agonist is administered before ingestion of food, optionally wherein the ASBTI and/or the enteroendocrine peptide enhancing agent and/or the FXR agonist is administered less than about 60 minutes or less than about 30 minutes before ingestion of food.

31. The method of claim 1, wherein the ASBTI and/or the enteroendocrine peptide enhancing agent and/or the FXR agonist is administered orally.

32. The method of claim 1, wherein the ASBTI and/or the enteroendocrine peptide enhancing agent and/or the FXR agonist is administered as an ileal-pH sensitive release or an enterically coated formulation.

33. The method of claim 1, wherein the enteroendocrine peptide enhancing agent and/or the FXR agonist is administered rectally.
